(19) United States
(12) Patent Application Publication
(10) Pub. No.: US 2007/0021495 A1
(43) Pub. Date: Jan. 25, 2007

(54) SULFONAMIDES AS SELECTIVE ESTROGEN RECEPTOR

(76) Inventors: John A. Katzenellenbogen, Urbana, IL (US); Benita S. Katzenellenbogen, Urbana, IL (US); Dennis R. Compton, Champaign, IL (US)

Correspondence Address: GREENLEE WINNER AND SULLIVAN P C, 4875 PEARL EAST CIRCLE, SUITE 200, BOULDER, CO, 80301, US

(21) Appl. No.: 11/459,917

(22) Filed: Jul. 25, 2006

Related U.S. Application Data
(60) Provisional application No. 60/702,151, filed on Jul. 25, 2005.

(51) Int. Cl. A61K 31/381; A61K 31/34; A61K 31/195; A61K 31/18; C07D 333/32; C07D 307/02; C07C 303/00

(52) U.S. Cl. 514/445; 514/471; 514/602; 514/562; 549/65; 549/475; 564/86

(57) ABSTRACT

Compounds, pharmaceutically acceptable salts, stereoisomers and prodrugs thereof, that are ER ligands and particularly to such compounds that are ER beta-selective and/or ER beta-specific ligands. Compounds herein include certain compounds which are ER beta-selective agonists. Compounds herein include ER beta-selective agonists which exhibit minimal agonist or antagonist effect on ER alpha. Compounds of the invention include those of formula I: 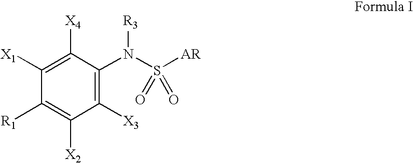
and any pharmaceutically acceptable salts, stereoisomers and prodrugs thereof wherein AR, R₁, R₃, and X₁—X₄ are as defined hereinabove.

### CROSS-REFERENCE TO RELATED APPLICATIONS

This application claims the benefit of U.S. Provisional application Ser. No. 60/702,151, filed Jul. 25, 2005 which is incorporated by reference herein in its entirety.

### STATEMENT REGARDING U.S. GOVERNMENT FUNDING

This invention was made through funding from the United States government through National Institutes of Health grant numbers PHS 5R37 DK 15556, PHS 5R37 CA 25836, and 5R01 CA 18119. The United States Government has certain rights in this invention.

### BACKGROUND OF THE INVENTION

This invention relates generally to estrogen receptor (ER) ligands, and particularly to ligands that exhibit subtype selective differences in ligand binding, transcriptional potency or efficacy for ER beta.

The estrogen receptor (ER), a member of the nuclear hormone receptor superfamily, mediates the activity of estrogens in the regulation of a number of important physiological processes, including the development and function of the female reproductive system and the maintenance of bone density and cardiovascular health. A variety of estrogen pharmaceuticals have been developed to regulate these processes or their pathological counterparts, including infertility, breast cancer, and osteoporosis. Estrogen pharmaceuticals have, for example, been developed for use as agents for regulating fertility, preventing and controlling hormone-responsive breast cancer, and menopausal hormone replacement. While the stimulation of processes in certain tissues has important health benefits, the stimulation of other tissues, such as the breast and uterus, can increase the risk of cancer at these sites. Intriguingly, some pharmaceutical agents, such as tamoxifen, act as antagonists in some tissues, such as the breast and uterus, while acting as agonists in other tissues, such as the liver and vasculature. See, for example, Grese, T. A. et al. (1997) Proc. Natl. Acad. Sci. USA, 94:14105-14110.

ER is a transcription factor that binds to specific estrogen response elements in the promoter region of estrogen-regulated genes and whose activity for transcription is modulated by the estrogen ligands (Katzenellenbogen, J. A. and Katzenellenbogen, B. S. (1996) Chem. Biol., 3:529-536). The capacity of ER-ligand complexes to activate gene transcription is mediated by a series of co-regulator proteins (Horwitz, K. B. et al. (1996) Mol. Endocrinol., 10:1167-1177). These co-regulators have interaction functions that tether ER to the RNA polymerase 11 pre-initiation complex, as well as enzymatic activities to modify chromatin structure (Glass, C. K. et al. (1997) Curr. Opin. Cell. Biol., 9:222-232).

The differential responses observed have raised the interesting issue of tissue-, cell-, and gene-specific activity of estrogens which is based on the ligand, the receptor, and/or the effector site and has been termed “tripartite receptor pharmacology”. (Katzenellenbogen, J. A.; O'Malley, B. W.; Katzenellenbogen, B. S. (1996) Mol. Endocrinol. 10, 119-131.) Each cell type and each gene presents to an ER(subtype)-ligand complex a unique combination of these effector components—various estrogen response elements and co-regulators—that appear to underlie, in part, the cell and gene selectivity of various estrogens. Extensive efforts are being expended to develop ligands which selectively antagonize undesirable estrogenic effects such as the stimulation of breast cancer, while promoting positive estrogen effects for bone and cardiovascular maintenance.

Tamoxifen, the ER ligand most commonly employed in hormonal therapy for estrogen-positive breast cancer (Jordan, V. C. (1995) Breast Cancer Res. Treat. 36:267-285), is a mixed agonist/antagonist for ER receptors. This drug exhibits a number of side effects when used in breast cancer therapy. The level of agonist-antagonist activity of tamoxifen is variable and tissue dependent (Katzenellenbogen, B. S. (1996) Biol. Reprod. 54:287-293 and Katzenellenbogen, J. A. et al. (1996) Mol. Endocrinol., supra). Tamoxifen may increase the incidence of liver and uterine cancer (Davidson, N. (1995) New Eng. J. Med., 332.:1638-1639 and Katzenellenbogen, B. S. (1991) J. Natl. Cancer Inst. 83:1434-1435). In contrast, the stimulatory effects of tamoxifen in bone cells can be beneficial for the prevention of osteoporosis in postmenopausal women (Katzenellenbogen, B. S. (1996) Biol. Reprod., supra). Pure antiestrogens, such as ICI 164,384, which antagonizes estrogen fully in all tissues, also show promise for hormonal therapy for estrogen-positive breast cancer, but exhibit detrimental effects on other estrogen positive tissues (bone, central nervous system and the cardiovascular system). A selective endocrine profile, as yet not achieved, which effects the desired inhibitory response in targeted tumor cells, while avoiding detrimental inhibitory or stimulatory effects in other tissues, is preferred in a drug for use in hormonal therapy for estrogen-positive breast cancer.

It had been assumed that estrogen-related events were mediated by only one estrogen receptor. However, the discovery of a second estrogen receptor (ERbeta) (Mosselman, S.; Polman, J.; Dijkema, R. (1996) FEBS Lett., 392, 49-53; Kuiper, G. G. J. M. et al. (1996) Proc. Natl. Acad. Sci. USA, 93, 5925-5930) indicates that tissue- and cell-selectivity of certain estrogens may be due, in part, to their mediation through ERbeta separate from, or in conjunction with, the classical estrogen receptor (ERalpha). This possibility has been supported by the difference in tissue distribution between ER alpha and ER beta (Mosselman, S.; Polman, J.; Dijkema, R. (1996) FEBS Left. supra; Kuiper, G. G. J. M. et al. (1997) Endocrinology 138:863-870; Saunders, P. T. K. et al. (1997) J. Endocrinol. 154:R13-R16; Register, T. C.; Adams, M. R. (1998) J. Steroid Biochem. Mol. Biol. 64:187-191.)

ER alpha and ER beta exhibit complex tissue distributions. Certain tissues may contain only (or predominately) ER alpha or ER beta and other tissues may contain a mixture of both ER alpha and ER beta. Tissues that exhibit high levels of ER beta include, for example, prostate, testes, ovaries, gastrointestinal tract, lung, bladder, hematopoetic and central nervous systems, and certain regions of the brain, whereas ER alpha predominates in the uterus, breast, kidney, liver and heart. Many tissues contain both ER alpha and ER beta, such as breast, epididymis, thyroid, adrenal, bone, and certain other regions of the brain. Furthermore, it has been shown that the pharmacology of traditional ER agonists and antagonists is reversed for ER beta in the context of certain ER effector sites. (Paech, K. et al. (1997) Science 277:1508-1510.)

ER selective ligands of this invention are sulfonamides, particularly N-alkyl sulfonamides. Sulfonamides as a general class have been shown to work well as drugs and potential drugs (Supuran, C. T.; Scozzafava, A.; Casini, A. Carbonic anhydrase inhibitors. Med. Res. Rev. 2003, 23,146-189; Scozzafava, A.; Owa, T.; Mastrolorenzo, A.; Supuran, C. T. Anticancer and antiviral sulfonamides. Curr. Med. Chem. 2003, 10, 925-953; Rao, P. N. P.; Amini, M.; Li, H.; Habeeb, A. G.; Knaus, E. E. Design, Synthesis, and Biological Evaluation of 6-Substituted-3-(4-methanesulfonylphenyl)-4-phenylpyran-2-ones: A Novel Class of Diarylheterocyclic Selective Cyclooxygenase-2 Inhibitors. J. Med. Chem. 2003, 46, 4872-4882; Bouchain, G.; Leit, S.; Frechette, S.; Khalil, E. A.; Lavoie, R. et al. Development of Potential Antitumor Agents. Synthesis and Biological Evaluation of a New Set of Sulfonamide Derivatives as Histone Deacetylase Inhibitors. J. Med. Chem. 2003, 46, 820-830.). Sulfonamides have, for example, been found to be useful as carbonic anhydrase inhibitors (Supuran et al. 2003); histone deacetylase inhibitors (Vaisburg, A.; Bernstein, N.; Frechette, S.; Allan, M.; Abou-Khalil, E. et al. (2-Amino-phenyl)-amides of omega-substituted alkanoic acids as new histone deacetylase inhibitors. Bioorg. Med. Chem. Left. 2004, 14, 283-287); cyclooxyenase-2 inhibitors (Rao et al. 2003; Habeeb, A. G.; Rao, P. N. P.; Knaus, E. E. Design and Synthesis of Celecoxib and Rofecoxib Analogues as Selective Cyclooxygenase-2 (COX-2) Inhibitors: Replacement of Sulfonamide and Methylsulfonyl Pharmacophores by an Azido Bioisostere. J. Med Chem. 2001, 44, 3039-3042, Habeeb, A. G.; Rao, P. N. P.; Knaus, E. E, Design and Synthesis of 4,5-Diphenyl-4-isoxazolines: Novel Inhibitors of Cyclooxygenase-2 with Analgesic and Antiinflammatory Activity. J. Med. Chem. 2001, 44, 2921-2927) and as antitumor agents (Scozzafava et al. 2003; and Bouchain et al. 2003).

Published U.S. application 2004/0110767 relates to acyclic amide and sulfonamide ligands for the estrogen receptor of formula: 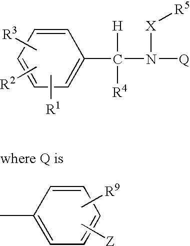
or a six-membered heteroaryl ring containing one or two nitrogen atoms, optionally substituted with R⁹ and/or Z, where X is CO or SO₂; R^{1,} R², R³, and R⁹ are hydrogen, hydroxy, halogen, cyano, C1-C6 alkyl, optionally substituted with 1-3 fluorine atoms and C1-C6 alkoxy, optionally substituted with 1-3 fluorine atoms; R⁴ is hydrogen or C1-C6 alkyl; R₅ is C1-C7 alkyl, optionally substituted with from 1-6 halogens; C2-C6 alkenyl; C2-C6 alkenyl-M or —(CH₂)ₙ-M, where n is 0-5; M is:

(i) a fully saturated 3- to 8-membered ring, or a partially saturated, or fully saturated 5- to 8-membered ring, optionally having from 1-4 heteroatoms, which are independently selected from the group consisting of oxygen, nitrogen, and sulfur; or

(ii) a bicyclic ring comprising two fused, partially-saturated, fully-saturated, or fully-unsaturated 5- or 6-membered rings, optionally having from 1-4 heteroatoms, which are independently selected from the group consisting of oxygen, nitrogen and sulfur; wherein M is optionally substituted with from 1-3 substituents independently selected from the group consisting of hydroxy; halogen; cyano; nitro; formyl; amino; carbamoyl; thiol; —(C1-C6)alkyl or —O(C1-C6)alkyl, optionally substituted with from 1-5 halogen atoms; —(C3-C8)cycloalkyl or phenyl, optionally substituted with from 1-3 halogen atoms; —SO(C1-C6)alkyl or —SO₂(C1-C6)alkyl, optionally substituted with from 1-5 halogen atoms; —S(C1-C6)alkyl, optionally substituted with from 1-5 halogen atoms; —(C1-C4)alkoxycarbonyl; —(C1-C6)alkyl-(C3-C8)cycloalkyl; —(C0-C4)sulfonamido; mono-N— or di-N,N—(C1-C4)alkylcarbamoyl; mono-N or di-N,N—(C1-C4)alkylamino-SO₂; mono-N or di-N,N—(C1-C4)alkylamino; —(C1-C8)alkanoyl; —(C1-C4)alkanoylamino; or —(C1-C4) alkoxycarbonylamino; and

Z is —O(CH₂)ₙ—NRₐR_{b}; —(CH₂)ₙ—NRₐR_{b}; —CH═CH—C(O)—NRₐR_{b}; —(CH₂)ₙ—COOH; —CH═CH—COOH; —O(C1-C6)alkyl; —CH═CH—C(O)O(C1-C6)alkyl; and —(CH₂)ₙ—OH; wherein each n is 0-5 inclusive, provided that when Z is —O—(CH₂)ₙ—NRₐR_{b}; n is 2-5; Rₐ and R_{b} are, independently, hydrogen; —(C1-C6)alkyl; —(CH₂)ₙ—(C3-C8)cycloalkyl; —(CH₂)ₙ-5-OH; —(CH₂)ₙ-phenyl; —(CH₂)ₙ-heteroaryl; —(CH₂)ₙ-heterocycloalkyl; and 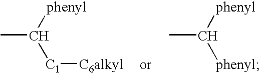
wherein each n is 0-5 inclusive, and wherein the cycloalkyl, phenyl, heteroaryl, and heterocycloalkyl are optionally substituted with from 1-3 substituents independently selected from the group consisting of hydroxy; halogen; cyano; nitro; amino; carbamoyl; —(C1-C6)alkyl or —O(C1-C6)alkyl, optionally substituted with from 1-5 halogen atoms; —(C1-C3)alkyl-O(C1-C3)alkyl; —(C1-C4)OH; carboxylate; —(C1-C3)phenyl; —(C3-C8)cycloalkyl; phenyl, optionally substituted with from 1-3 halogen atoms; —SO(C1-C6)alkyl or —SO₂(C1-C6)alkyl, optionally substituted with from 1-5 halogen atoms; —S(C1-C6)alkyl, optionally substituted with from 1-5 halogen atoms; —(C1-C4)alkoxycarbonyl; —(C1-C6)alkyl-(C3-C8)cycloalkyl; sulfonamido; —(C1-C4)alkylsulfonamido; mono-N— or di-N,N—(C1-C4)alkylcarbamoyl; mono-N or di-N,N—(C1-C4)alkylamino-SO₂; mono-N or di-N,N—(C1-C4)alkylamino; —(C3-C8)alkanoyl; —(C1-C4)alkanoylamino; or —(C1-C4)alkoxycarbonylamino; or
Rₐ and R_{b}, taken together with the nitrogen atom to which they are attached, form a 3- to 7-membered heterocycloalkyl ring having from 1-2 heteroatoms which are independently selected from the group consisting of nitrogen, oxygen, and sulfur; or a 5- to 7-membered ring fused to a phenyl ring, wherein the 3- to 7-membered heterocycloalkyl ring, or the 5- to 7-membered ring fused to a phenyl ring is optionally substituted with from 1-3 substituents independently selected from the group consisting of hydroxy; halogen; cyano; nitro; amino; carbamoyl; —(C1-C6)alkyl or —O(C1-C6)alkyl, optionally substituted with from 1-5 halogen atoms; —(C1-C3)alkyl-O(C1-C3)alkyl; —(C1-C4)OH; carboxylate; —(C1-C3)phenyl; —(C3-C8)cycloalkyl; phenyl, optionally substituted with from 1-3 halogen atoms; —SO(C1-C6)alkyl or —SO₂(C1-C6)alkyl, optionally substituted with from 1-5 halogen atoms; —S(C1-C6)alkyl, optionally substituted with from 1-5 halogen atoms; —(C1-C4)alkoxycarbonyl; —(C1-C6)alkyl-(C3-C8)cyclo alkyl; —(C0-C4)sulfonamido; —(C1-C4)cycloalkylsulfonamido; mono-N— or di-N,N—(C1-C4)alkylcarbamoyl; mono-N or di-N,N—(C1-C4)alkylamino-SO₂; mono-N or di-N,N—(C1-C4)alkylamino; —(C1-C8)alkanoyl; —(C1-C4) alkanoylamino; or —(C1-C4)alkoxycarbonylamino.

Q in the above formula is defined to be certain aryl and heteroaryl rings. However, R⁵ in the above formula does not include 5- or 6-membered rings, such as phenyl rings, thiophene rings or furan rings. The compounds of this reference are described as ER alpha or ERbeta selective. None of the compounds of this reference are described as ER agonists or ER antagonists. None of the compounds of this reference are described as ERbeta-selective agonists.

Stauffer S. R. et al. (2000) Biorganic & Medicinal Chemistry 8:1293-1316 relates to acyclic amides as estrogen receptor ligands. Compounds disclosed include certain carboxamides, thiocarboxamides and sulfonamides. Sulfonamides of structure: 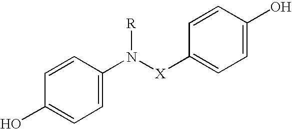
where X is SO₂, R is ethyl, n-butyl and benzyl were found to have RBA (relative binding affinities, where estradiol is 100) of 0.23, 0.13 and 0.053, respectively, measured in lamb uterine cyctosol. Corresponding or related thiocarboxamides (where X is C═S, with various R) exhibited generally higher RBA (lamb cytosol) compared to sulfonamides. Individual ER alpha and ER beta affinities were not measured for these sulfonamides because of the low RBA values obtained. The affinity of certain carboxamides (where X is C═O, with various R) and thiocarboxamides toward ER alpha and ER beta was assessed. All carboxamides and thiocarboxamides tested were found to be ER alpha selective and full or nearly full agonists on ER alpha.

Osawa, Y., Synthesis and Estrogenic Activity of p-Methoxybenzenesulfon-p-anisidide and Its N-Derivatives, Nippon Kagaku Zasshi, 84, 134 (1963); Chem. Abst., 59, 13863c (1963) reports the synthesis and estrogenic activity of p-methoxybenzenesulfon-p-anisidide and its N-derivatives: 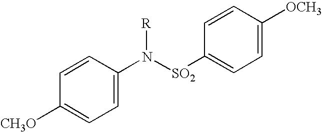
where R is H (compound II), methyl (compound III), ethyl (Compound IV), n-propyl (compound V), i-propyl (compound VI), benzyl (compound VII), and acyl (unstable in air). A related compound of formula p-CH₃O—C₆H₄—SO₂N(Phenyl)₂ (compound VIII) was also prepared. Estrogenic activities were tested with ovariectomized mice and results given as follows (compd., dose in (micrograms) and percentage of animals which responded: II, 50, 100; III, 25, 80; IV, 50, 100; V, 50, 60; V, 100, 80; VI, 100, 60; VII, 500, 40; VIII, 250, 60. The weak estrogenic activity of compound VI was attributed to the unfavorable molecular structure. Compound II was reported to have some activity against Candida albicans.

In a related reference: Osawa, Y., Synthesis and Estrogenic Activity of 4,4′-Disubstituted Benzenesulfonanilides, Nippon Kagaku Zasshi, 84, 137 (1963); Chem. Abst., 59, 13863a (1963) the synthesis and estrogenic activity of compounds of formula: 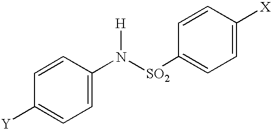
where X is Cl, OCH₃, Br or NH₂ and Y is NH₂ or AcNH or X is Cl, Br or NH₂ and Y is OCH₃ was reported. Estrogenic activities were tested with ovariectomized mice and results given as follows: Compd Y, X ( dose in (micrograms) and percentage of animals which responded): AcNH, Cl (1000,20); ACNH, OCH₃ (1000, 40); AcNH, Br(1000, 80); NH₂, Cl(1000, 80) NH₂, OCH₃ (100, 100); NH₂, Br(1000, 80); NH₂, NH₂ (500, 80), OCH₃, Cl (100, 100), OCH₃, Br (100, 100) and OCH₃, NH₂ (100, 60). The compound where Y is NH₂ and X is OCH₃ was also reported to exhibit some activity against tubercle bacteria and Escherichia coli.

U.S. Pat. No. 7,045,539 (issued May 16, 2006) relates to certain benzoxazole compounds having a base structure: 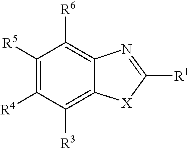
where X is O or S which are reported to be ER beta-selective ligands. In this formula: R¹ is C₁₋₈ alkyl, phenyl, benzyl or a 5- or 6-membered ring heterocycle containing 1, 2 or 3 heteroatoms each independently selected from O, N and S and additionally having 0 or 1 oxo groups and 0 or 1 fused benzo rings, wherein the C₁₋₈ alkyl, phenyl, benzyl or heterocycle is substituted by 0, 1, 2 or 3 substituents selected from —R^{a}—OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R.^{a}, —NR.^{a}S(═O)₂R^{a}, —C(═O)R^{a}, —S(═O)R^{a}, —S(═O)₂R^{a}, halogen, cyano, nitro and C₁₋₃ haloalkyl;
R³ is —R^{a}, —OR^{a}, —SR^{a}, —NR^{a}R^{a}, —CO₂R^{a}, —OC(═O)R^{a}, —C(═O)NR^{a}R^{a}, —NR₂C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁₋₃haloalkyl; or R³ is C₁₋₃ alkyl containing 1 or 2 substituents selected from —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano and nitro; R⁴ is —Rₐ, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro or C₁₋₃haloalkyl; R⁵ is —Rₐ, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro or C₁₋₃haloalkyl;
R⁶ is —R^{a}, —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano, nitro and C₁₋₃haloalkyl; or R⁶ is C₁₋₃alkyl containing 1 or 2 substituents selected from —OR^{a}, —SR^{a}, —NR^{a}R^{a}, —CO₂R^{a}, —OC(═O)R^{a}, —C(═O)NR^{a}R^{a}, —NR^{a}C(═O)R^{a}, —NR^{a}S(═O)R^{a}, —NR^{a}S(═O)₂R^{a}, —C(═O)R^{a}, —S(═O)R^{a}, —S(═O)₂R^{a}, halogen, cyano and nitro; and Rₐ is H, C₁₋₆alkyl, C₁₋₃haloalkyl, phenyl or benzyl; and pharmaceutically acceptable salts thereof.

U.S. published patent application 2006/0116364 (published Jun. 1, 2006) relates to certain compounds that are reported to be selective estrogen receptor modulators. The patent application contains a very broad structure for such modulators, however the synthetic examples appear to focus on a narrower range of compounds. Estrogen receptor binding affinities for a number of compounds are given in Table 1. A comparison of agonist and antagonist activity of certain compounds is presented in Table 2. Certain animal model results are provided for two compounds: 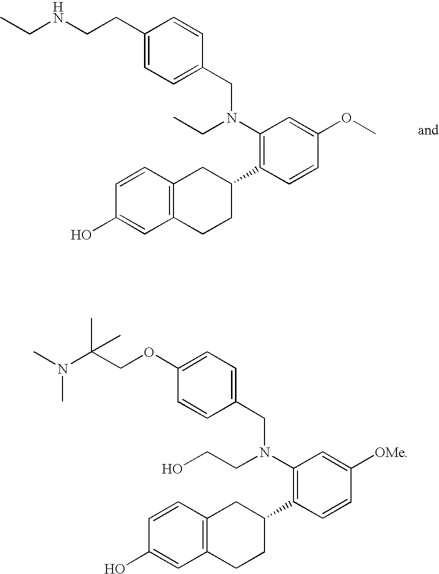
It appears that no sulfonamides were exemplified in the synthetic or test examples.

Japanese patent application JP 02145560A2 (published 1990) reports compounds of formula: HOC₆H₄NR1 SO₂R2, wherein R1 is H or a C1-C4 alkyl and R2 is a C2-C12 alkyl or an aryl group. An example compound is N-(4-hydroxyphenyl)-butane sulfonamide. The compounds are reported to be useful as developers for thermal and pressure-sensitive recording materials.

Japanese patent application JP 01141786A2 and Japanese published examined application JP 08002697B4 (published 1989) report compounds of formula: 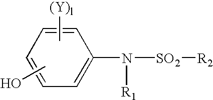
where:
R₁ is H, a C1-C8 alkyl, a cycloalkyl group, an aryl group, an aralkyl group and an alkenyl group, R₂ is a C1-C10 alkyl group, a cycloalkyl group, an aryl group, an aralkyl group or an alkenyl group, Y is a halogen atom, a nitryl group or a C1-C4 alkyl group and l is an integer from 0-2. These compounds are reported to be useful in certain compositions for recording material.

Shafer, S. J and Closson, W. D. J. Organic Chem. (1975) 40(7): 889-92 report base-promoted rearrangements of certain arenesulfonamides. Rearrangements reported were those of compounds of formula: 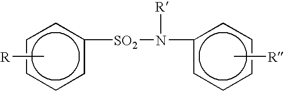
where
R═R″″H, R′═CH₃;
R=p-CH₃, R′═CH₃, and R″=pCH₃O;
R═R′═H, R′═CH₃CH₂;
R=p-CH₃, R═CH₃CH₂, and R″═H;
R=p-CH₃O; R═CH₃CH₂, and R″═H;
R═R″═H, R′═C₆H₅;
R=p-(CH₃)₂N, R′═CH₃, R″═H;
R=p-CH₃O, R′═CH₃, R″═H;
R=p-CH₃O, R′═CH₃, and R″=p-CH₃O;
R=o-CH₃, R′═CH₃, R″═H; and
R=p-CH₃; R′═CH₃, R″═H.

U.S. Pat. No. 6,521,658, published U.S. application 2003096856 and published PCT application WO 2000073264 relate to sulfonamides as cell proliferation inhibitors of formula (taken from U.S. Pat. No. 6,521,658): 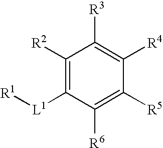
where:
L¹ can, among other groups, be —R⁷N—SO₂— or —SO₂—NR⁷— where R⁷ is selected from the group consisting of: hydrogen, hydroxy, amidinyl, a nitrogen-protecting group, or optionally substituted alkanoyl, alkyl, alkenyl, alkynyl, cycloalkyl, cycloalkylalkyl, cycloalkenyl, cycloalkenylalkyl, aryloyl, or alkoxy groups where the optional substituents are one, two, or three substituents independently selected from the group consisting of hydroxyl, halo, cyano, azido, carboxy, amidinyl, alkyl, aryl, oxo, or heteroaryl and heterocycloalkyl which can be optionally substituted with 1, 2, or 3 substituents independently selected from the group consisting of alkyl and a nitrogen protecting group, —NRcRd, wherein Rc and Rd are independently selected from the group consisting of hydrogen, alkyl, aryl, and alkoxyalkyl, and -(alkylene)-NRcRd, heterocycloalkyloyl, wherein the heterocycloalkyloyl can be optionally substituted with 1, 2, or 3 substituents independently selected from the group consisting of alkyl and a nitrogen protecting group, and —(CH₂)ₓNR_{A}R_{B}, wherein x is 0-6, and R_{A} and R_{B} are independently selected from the group consisting of hydrogen, alkyl, alkenyl, alkynyl, cycloalkyl, cycloalkylalkyl, cycloalkenyl, and cycloalkenylalkyl;
R¹ is aryl or heteroaryl, wherein the aryl or the heteroaryl can be optionally substituted with 1, 2, 3, 4, or 5 substituents independently selected from the group consisting of oxo, azido, carboxy, carboxaldehyde, cyano, halo, hydroxy, nitro, perfluoroalkyl, perfluoroalkoxy, alkyl, alkenyl, alkynyl, alkanoyloxy, alkoxycarbonyl, cycloalkyl, cycloalkylalkyl, cycloalkenyl, cycloalkenylalkyl, alkanoyl, alkoxy, cycloalkoxy, aryloxy, heteroaryloxy, thioalkoxy, alkylsulfinyl, alkylsulfonyl, —NR⁸R⁹, wherein R⁸ and R⁹ are independently selected from the group consisting of hydrogen, alkyl, arylalkyl, and alkanoyl, wherein the alkanoyl can be optionally substituted with 1 or 2 substituents independently selected from the group consisting of: halo, hydroxy, and —NR¹⁰R¹¹, wherein R¹⁰ and R¹¹ are independently hydrogen or alkyl, and —SO₂NR⁸R⁹, wherein R⁸ and R⁹ are defined above;
R² and R⁶ are independently selected from the group consisting of hydrogen, alkyl, alkoxy, thioalkoxy; and hydroxy; and
R³, R⁴, and R⁵ are independently selected from the group consisting of alkyl, alkoxy, thioalkoxy, and hydroxy; with the proviso that combinations wherein L′ is —NR⁷SO₂— and R′ is: unsubstituted or substituted 1H-indoly-7-yl, phenyl which is 2-monosubstituted with —NR⁸R⁹, pyrid-3-yl which is 2-monosubstituted with —NR⁸R⁹, or pyrimidin-5-yl which is 4-monosubstituted with —NR⁸R⁹, are excluded.

U.S. Pat. No. 6,683,201 and published U.S. application 2002038025 relate to aniline derivatives for treatment of 2,3-oxidosqualene-lanosterol cyclase associated diseases of formula: 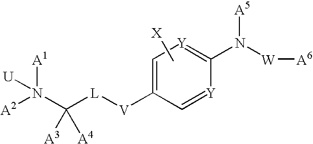
wherein
U is O or a lone pair;
Y is C or N;
V is O, S, NR′, —CH₂—, —CH═CH—, or —C═C—, if Y is C, or —CH₂—, —CH═CH—, —C═C—, if Y is N;
W is CO, COO, CONR′, CSO, CSNR′, SO₂, or SO₂NR′;
L is lower-alkylene, lower-alkenylene, or a single bond;
A¹ is H, lower-alkyl, or lower-alkenyl;
A² is lower-alkyl, cycloalkyl, cycloalkyl-lower-alkyl, lower-alkenyl, or lower-alkynyl, each unsubstituted or substituted by R₂;
A³, A⁴ are hydrogen or lower-alkyl, or
A¹ and A² or A¹ and A³ are bonded to each other to form a ring and -A¹-A²- or -A¹-A³- are lower-alkylene or lower-alkenylene, each unsubstituted or substituted by R², or are lower-alkylene or lower-alkenylene, each unsubstituted or substituted by R², in which one —CH₂— group of -A¹-A²- or -A¹-A²- is replaced by NR³, S, or O;
A⁵ is lower-alkyl;
X is hydrogen or one or more halogen substituents;
A⁶ is lower-alkyl, cycloalkyl, cycloalkyl-lower-alkyl, heterocycloalkyl-lower-alkyl, lower alkenyl, lower-alkadienyl, aryl, aryl-lower-alkyl, heteroaryl, or heteroaryl-lower-alkyl;
R² is hydroxy, hydroxy-lower-alkyl, lower-alkoxy, N(R4R5), or lower-alkoxycarbonyl; and
R¹, R³, R⁴, R⁵ and R⁶, independently from each other, are hydrogen or lower-alkyl, or a pharmaceutically acceptable salt or pharmaceutically acceptable ester thereof. Methods for making the above-listed compounds and intermediates in such methods are reported.

U.S. Pat. No. 6,605,635 and published PCT application WO 2001042204 relate to N-substituted benzyl or phenyl aromatic sulfonamides as antiarrhythmics of general formula: 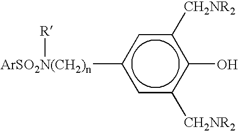
where Ar represents phenyl or naphthyl optionally substituted with an alkyl, an alkoxy, a nitro, a halogen or a substituted amino group;
n=0 or 1;
NR₂ represents N(CₓH₂ₓ₊₁)₂, 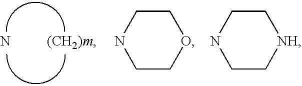
   and the like, wherein x=1 or 2, and m=4, 5 or 6.

U.S. Pat. No. 6,586,617 and published patent application US 2004023938 relate to aryl-or heterocyclylsulfonamide derivates as agricultural and horticultural microbiocides having the following formula or salts thereof. 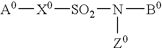
wherein, A⁰ is an aryl group which may be substituted, or a heterocyclic group which may be substituted; X⁰ is a chemical bond, a methylene group, which may be substituted, or a vinylene group which may be substituted; B⁰ is a heterocyclic group which may be substituted or an aryl group which may be substituted; Z⁰ is a hydrocarbon group which may be substituted, an acyl group which may be substituted, a formyl group, an amino group, which maybe substituted,
—N═CR1R2 (wherein R1 and R2 is a hydrogen atom or a hydrocarbon group which may be substituted), a cyclic amino group, —OR′(wherein R3 is a hydrogen atom, a hydrocarbon group, which may be substituted, an acyl group, which may be substituted, a formyl group, or an alkylsulfonyl group, which may be substituted, or a —S(O)nR4 (wherein n represents an integer from 0 to 2, and R4 is a hydrogen atom or a hydrocarbon group which may be substituted) or salts thereof. The compounds are reported to have very strong microbiocidal activity, with low toxicity to human being and animals. The references relate generally to microbiocidal compositions for agricultural or horticultural use comprising a compound as above.

U.S. Pat. No. 5,905,156 relates to the preparation of benzopyran-6-sulfonamides as potassium channel opening agents having the formula: 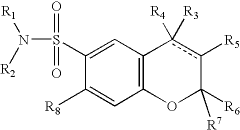
The reference reports intermediates in the synthesis of the compounds of the invention. Intermediates reported include compounds of formula: 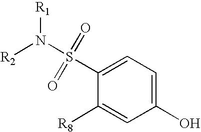
where R₁ is aryl, R₂ is H or C1-5 alkyl, or is C2-5 alkylene linked to R₁, and R₈ is hydrogen or C1-5 alkyl. A preferred group of potassium channel opening agents are reported to be those compounds where R₁ is phenyl, fluorophenyl, trifluoromethylphenyl, methoxyphenyl, or pyridyl; and/or R₂ is methyl, ethyl, or H; or R₁ and R₂ together with N form a 1,2,3,4-tetrahydroquinolin-1-yl. Preferred compounds are reported to be those in which R₁ is phenyl; R₂ is H or methyl, or is trimethylene linked to R₁, so that R₁ and R₂ together with N form a 1, 2, 3, 4-tetrahydroquinolin-1-yl group and R₈ is hydrogen.

Pokrywiecki, S et al. (1973) Crystal Structure Communications 2(1) 67-72 reports the crystallographic parameter of the compound p-ethoxybenzenesulfon-N-isopropyl-p-anisidide (C₁₇H₂₁NO₄S):

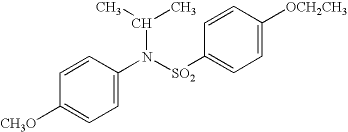

While a number of sulfonamides useful in pharmaceutical applications have been described. Sulfonamides which are ER subtype selective and ER subtype specific ligands have not been described.

### SUMMARY OF THE INVENTION

The invention relates to compounds, pharmaceutically acceptable salts, stereoisomers and prodrugs thereof, that are ER ligands and particularly to such compounds that are ER beta selective and/or ER beta specific ligands. In certain embodiments, the invention relates to compounds which are ER beta selective agonists. In specific embodiments, the invention relates to compounds pharmaceutically acceptable salts, stereoisomers and prodrugs thereof which are ER beta selective agonists and which exhibit minimal agonist or antagonist effect on ER alpha.

The invention relates generally to compounds of formula I: 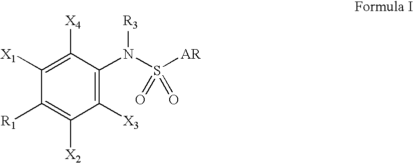
and pharmaceutically acceptable salts, stereoisomers and prodrugs thereof wherein AR, R₁, R₃, and X₁—X₄ are as defined below.

The invention further relates to novel sulfonamides of formula I as well as novel salts, stereoisomers, and/or prodrugs thereof. In a specific embodiment, the invention relates to compounds of formula I as well as salts, stereoisomers, and/or prodrugs thereof for which no enabling disclosure is given in any prior art reference and particularly for which no enabling disclosure is given in any prior art reference cited herein.

The invention further relates to novel sulfonamides of formulas II, III and/or IV (below) as well as novel salts, stereoisomers, and/or prodrugs thereof. In a specific embodiment, the invention relates to compounds of formulas II-IV as well as salts, stereoisomers, and/or prodrugs thereof for which no enabling disclosure is given in any prior art reference and particularly for which no enabling disclosure is given in any prior art reference cited herein.

The invention provides a method for selectively regulating the expression of one or more genes in a mammalian cell or in mammalian tissue, the expression of which are affected through an estrogen receptor (ER), and particularly through ER beta, which comprises the step of contacting the cell or tissue with an amount, or a combined amount, of one or more compounds of formula I or salts, stereoisomers or prodrugs thereof sufficient to affect the expression of one or more genes in the cell or tissue. In a preferred embodiment, the invention provides a method for selectively regulating the expression of one or more genes in a mammalian cell or in mammalian tissue, the expression of which are affected through ER beta, which comprises contacting the cell or tissue with an amount, or a combined amount, of one or more compounds of formula I or formulas II-IV or salts, stereoisomers or prodrugs thereof sufficient to regulate expression of one or more genes in the cell or tissue that are affected through ER beta, but wherein the one or more compounds do not exhibit any significant affect on expression of any gene in the cell or tissue the expression of which is affected through ER alpha. This method is particularly applicable to cells or tissue in which the expression of one or more genes is affected through either or both of ER alpha or ER beta. This method is particularly applicable to cells or tissue containing one or more genes, the expression of which is increased or enhanced through ER beta. This method is also applicable to cells or tissue containing at least one gene, the expression of which is increased or enhanced through ER beta, and at least one gene, the expression of which is decreased or inhibited through ER alpha.

Certain compounds of formulas I-IV and/or salts, stereoisomers, and/or prodrugs thereof are useful for exerting agonist effects through ER beta without affecting or at least without any significant effect on estrogen action through ER alpha.

The invention also provides pharmaceutical or therapeutic compositions comprising one or more of the compounds of formula I, or salts, stereoisomers or prodrugs thereof and methods for the treatment of diseases, disorders, conditions or symptoms affected through an estrogen receptor (ER), particularly those diseases, disorders, conditions or symptoms affected through ER beta wherein a therapeutically effective amount, or combined amount, of one or more of the compounds of formula I-IV or salts, stereoisomers or prodrugs thereof is administered to a mammal in need of such treatment in an amount effective to affect expression of one or more genes the expression of which is regulated through ER, particularly through ER beta. More specifically, the amount or combined amount of compound administered is an amount effective to affect amelioration of the disease, disorder, condition or symptoms affected through ER beta.

In another embodiment the present invention provides the use of a compound of the formula I-IV or a pharmaceutically acceptable salt, stereoisomer or prodrugs thereof for the manufacture of a medicament for the treatment of a disease, disorder, condition or symptom affected through ER beta, more particularly in treating one or more of hyperplasia, breast cancer, infertility, inflammation, inflammatory bowel disease, cardiovascular disease, endocrine disorders, osteoporosis, depression, anxiety, and immune disorders.

The invention also provides methods in which a mammal in need of treatment for a disease, disorder, condition or symptom that is ameliorated by changing the expression level of a gene, the expression of which is regulated through ER beta, is treated by administering one or more ER beta agonist of formulas I-IV, or salts, stereoisomers or prodrugs thereof to the mammal. In such methods a therapeutically effective amount, or combined amount, of one or more of the compounds of formula I-IV or salts, stereoisomers or prodrugs thereof is administered to the mammal in an amount effective to affect expression of one or more genes the expression of which is regulated through ER beta. More specifically, the amount or combined amount of one or more compounds administered is an amount effective to affect amelioration of the disease, disorder, condition or symptoms affected through ER beta.

In a specific embodiment, the invention provides methods in which a mammal, in need of treatment for a disease, disorder, condition or symptom that is ameliorated by increasing or enhancing the expression of a gene, the expression of which is regulated through ER beta, is treated by administering one or more ER beta agonist of formula I to the mammal. In specific embodiments, the methods employ one or more ER beta selective agonists of formula I. In other specific embodiments, the methods employ one or more ER beta specific agonists of formula I.

In a specific embodiment, the invention provides methods in which a mammal, in need of treatment for a disease, disorder, condition or symptom that is affected by the expression of one or more genes, the expression of which are regulated through ER beta, is treated by administering one or more ER beta agonist of formula I in combination with one or more ER alpha selective antagonists to the mammal. In specific embodiments, the methods employ one or more ER beta selective agonists of formula I in combination with one or more ER alpha selective antagonists.

In more specific aspects of this invention, the compounds of formula I and/or salts, stereoisomers and/or prodrugs thereof are useful in the treatment of humans. In particular aspects of this invention, the compounds of formula I and/or salts, stereoisomers and/or prodrugs thereof are useful in the treatment of estrogen receptor related diseases, conditions and/or symptoms including among others, hyperplasia, breast cancer, infertility, inflammatory bowel disease, and osteoporosis. Additionally, the compounds of this invention and compositions containing them can provide antiproliferation effect, antiinflamatory effect, cardiovascular protection, and immune protection. The compounds and compositions of this invention further can provide benefit for treatment of endocrine disorders, inflammation, and depression.

The invention further provides pharmaceutical compositions which comprise a pharmaceutical carrier in combination with one or more compounds of formula I and/or a salt, stereoisomer, and/or prodrug thereof. The compound, salt, stereoisomer, and/or prodrug being present in the pharmaceutical composition in an amount effective for achieving the desired pharmaceutical effect, e.g., for achieving amelioration of a disease, disorder, condition or symptom that is affected by the expression level of one or more genes, the expression of which is affected through ER, and particularly through ER beta.

The invention also provides pharmaceutical or therapeutic compositions and methods for the treatment of diseases, disorders, conditions or symptoms affected through an estrogen receptor (ER), particularly those diseases, disorders, conditions or symptoms affected through ER beta wherein a therapeutically effective amount, or combined amount, of one or more of the compounds of formula I or salts, stereoisomers or prodrugs thereof is combined with a therapeutically effective amount, or combined amount, of one or more of ER alpha selective antagonists or salts, stereoisomers or prodrugs thereof and administered to a mammal in need of such treatment in an amount effective to affect expression of one or more genes the expression of which is regulated through ER, particularly through ER beta. More specifically, the amount or combined amount of compounds (salts, stereoisomers and/or prodrugs) administered is an amount effective to affect amelioration of the disease, disorder, condition or symptoms affected through ER beta. Pharmaceutical or therapeutic compositions include those which comprise one or more carriers in combination with the therapeutically active ingredients listed. Compositions in which one or more ER beta selective agonists are combined with one or more ER alpha selective antagonists are useful to provide very selective effects through ER beta.

Additional aspects of the invention are evident on consideration of the following drawings, detailed description and examples.

### BRIEF DESCRIPTION OF THE DRAWINGS

FIGS. 1A and B are graphs providing dose-response curves for certain ER ligands in Human endometrial cancer (HEC-1) cells transiently transfected with ER alpha or ER beta. Cells were transfected with 2x-pS2-ERE-Luc reporter gene, ER alpha (solid line) or ER beta (dashed line) and beta-galactosidase (as an internal control gene) and were then treated with ligand for 24 hours before assessing luciferase activity. Values are expressed as % of E2 activity at 1 nM±SEM from several independent experiments. Ligands assessed are :Estradiol (E2) (FIGS. 1A), FS-5 (FIG. 1B),

FIGS. 2A and 2B are graphs showing the lack of antagonism of FS-2 (A) and FS-5 (B) on estrogen-induced gene expression. HEC-1 cells were transfected with a 2x-pS2-ERE-Luc reporter gene, ERα (solid line) or ERβ (dashed line) and β-galactosidase (an internal control gene) and subjected to ligand treatment as indicated: FS-2+E2 at 1 nM (solid diamonds) and FS-5+E2 at 1 nM (solid squares), for 24 hours before assessing luciferase activity. Values are expressed as % of E2 activity at 1 nM and are the mean of duplicate determinations.

FIGS. 3A-3D illustrate regulation of endogenous gene expression by exemplary ligands of this invention. U2-OS cells stably expressing either ERα or ERβ as indicated were treated with FS-2 (A and C) or FS-5 (B and D) for 24 hours. Expression levels of cystatin D (A and B) or of GREB1 (C and D) mRNA were determined by quantitative PCR methods.

### DETAILED DESCRIPTION OF THE INVENTION

The invention relates to compounds, pharmaceutically acceptable salts, stereoisomers and prodrugs thereof, that are ER ligands and particularly to such compounds that are ER beta selective ligands. The invention also relates to methods of treating diseases, disorders, conditions and/or symptoms that are associated with ER receptors and the expression of ER receptor regulated genes. In specific embodiments, the invention relates to compounds which are ER beta selective agonists. In specific embodiments, the invention relates to compounds which are ER beta specific agonists which are ER beta selective agonists which exhibit minimal agonist or antagonist effect on ER alpha.

The invention relates generally to compounds of formula I: 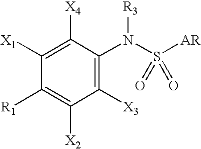
and pharmaceutically acceptable salts and prodrugs thereof as well as stereoisomers thereof wherein:
AR is an optionally substituted aryl group;
R₃ is an optionally substituted alkyl, alkenyl, alkynyl, benzyl, or phenyl group;
R₁ is a hydrogen, a halide, an optionally substituted alkyl, alkenyl, alkynyl, benzyl, or phenyl group, or a hydroxy, thiol, or optionally substituted alkoxy, thioalkoxy, or aryloxy group; and
X₁—X₄, independently of one another, are selected from the group consisting of hydrogens, halogens, optionally substituted alkyl groups, particularly C1-6 alkyl groups, optionally substituted alkoxy groups, particularly C1-C6 alkoxy groups, optionally substituted —CO—R groups, optionally substituted -SR groups, cyano groups, nitro groups, thiol groups, and hydroxy groups, where R is H, or an optionally substituted alkyl group, particularly C1-C6 alkyl group where optional substitution means substitution with one or more halogens, cyano groups, nitro groups, hydroxy groups, alkoxy groups, thiol groups, thioalkoxy groups, aryloxyl, N(R)′₂ groups, CON(R′)₂ groups or —COOR′ groups, where R′ is H or an optionally substituted alkyl group, particularly C1-C6 alkyl group and where R′ groups may be linked to form a cyclic alkyl group, wherein R₃ can be linked with X₃, X₄, or AR to form a 5, 6 or 7-member ring containing the N to which R₃ is bonded, which may be an aromatic ring and wherein the ring optionally contains one or two additional heteroatoms (e.g., N, O or S).

In specific embodiments, AR is

(1) an optionally substituted phenyl group:

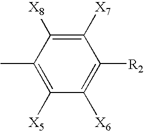

or

(2) an optionally substituted thiophene or furan group: 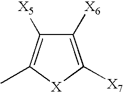
where X is S or O; and wherein:
X₅—X₈ are independently of one another defined as for X₁—X₄ above and wherein wherein R₃ can be linked with X₃, X₄, or AR through X₅, X₇ or X₃ to form a 5, 6 or 7-member ring as described above; and
R₂ is hydrogen, an OR group, a halogen, an optionally substituted alkyl group, particularly a C1-6 alkyl group, an optionally substituted alkoxy group, particularly a C1-C6 alkoxy group, an optionally substituted —CO—R group, an optionally substituted —SR group, a cyano group, a nitro group, a thiol group, and a hydroxy group, where R is H, or an optionally substituted alkyl group, particularly a C1-C6 alkyl group, where optional substitution means substitution with one or more halogens, cyano groups, nitro groups, hydroxy groups, thiol groups, thioalkoxyl groups, aryoxyl, —N(R′)₂ groups, —CON(R′)₂ groups or —COOR′ groups, where R′ is H or an optionally substituted alkyl group, particularly a C1-C6 alkyl group where R′ groups may be linked to form a cyclic alkyl group.

In specific embodiments, R₁ is OR, where R is hydrogen or an optionally substituted alkyl group, particularly an alkyl group having 1 to 6 carbon atoms.

In specific embodiments, the invention relates to compounds of the above formula where R₃ is a branched C1-C6 alkyl group, which may contain one or two double bonds (i.e., one or two alkenyl group) or a partially or fully halogenated C1-C6 alkyl group or a partially or fully halogenated C2-C5 alkenyl group. In more specific embodiments, the invention relates to compounds of the above formula where R₃ is a fully or partially fluorinated C1-C6 alkyl group or a fully or partially fluorinated C2-C6 alkenyl group. In additional specific embodiments, the invention relates to compounds of the above formula in which R₃ contains a trifluoromethyl group.

In specific embodiments, the invention relates to compounds of the above formula where AR is an optionally substituted phenyl group and wherein R₃ is a branched C1-C6 alkyl group, which may contain one or two double bonds (i.e., one or two alkenyl group) or a partially or fully halogenated C1-C6 alkyl group or a partially or fully halogenated C2-C5 alkenyl group. In more specific embodiments, the invention relates to compounds of the above formula where R₃ is a fully or partially fluorinated C1-C6 alkyl group or a fully or partially fluorinated C2-C6 alkenyl group. In additional specific embodiments, the invention relates to compounds of the above formula in which R₃ contains a trifluoromethyl group.

In specific embodiments, the invention relates to compounds of the above formula where R₃ is a branched C1-C6 alkyl group, or a partially or fully halogenated C1-C6 alkyl group and one or more of X1-X8 is a halogen. R₃ can more specifically be a fluorinated C1-C6 alkyl group, particularly a fluorinated alkyl group containing a trifluoromethyl group. In specific embodiments R₁ and R₂ are not both OCH₃ groups. In specific embodiments R₁ and R₂ are not both alkoxy groups. In specific embodiments when R₁ and R₂ are alkoxy groups (particularly OCH₃ groups), R₃ is not methyl, ethyl, n-propyl, i-propyl or benzyl groups.

In specific embodiments, R₃ is a C1-C6 cycloalkyl group. In specific embodiments, R₃ is a C1-C6 cycloalkyl group and R₁ and R₂ are OH, or OR where R is a C1-C6 alkyl which may be substituted. In specific embodiments, R₃ is a fluorinated C1-C6 cycloalkyl group. In specific embodiments, R₃is a fluorinated C1-C6 alkyl group and R₁ and R₂ are OH, or OR where R is a C1-C6 alkyl which may be substituted.

In other specific embodiments, the invention relates to compounds of the above formula where AR is an optionally substituted phenyl group and wherein R₃ is a branched C1-C6 alkyl group, or a partially or fully halogenated C1-C6 alkyl group and one or more of X1-X8 is a halogen. R₃ can more specifically be a fluorinated C1-C6 alkyl group, particularly a fluorinated alkyl group containing a trifluoromethyl group. In specific embodiments R₁ and R₂ are not both OCH₃ groups. In specific embodiments R₁ and R₂ are not both alkoxy groups. In specific embodiments when R₁ and R₂ are alkoxy groups (particularly OCH₃ groups), R₃ is not methyl, ethyl, n-propyl, i-propyl or benzyl groups.

In specific embodiments, AR does not carry an amino or amine substituent. In further specific embodiments, none of X₅—X₈ are amine or amino groups. In additional specific embodiments, X₈ is not an amine or amino group.

In specific embodiments of compounds herein when AR is a substituted phenyl group, when R₁ and R₂ are both C1-C3 unsubstituted alkoxy groups, R₃ is not a C1-C6 unsubstituted alkyl group. In other specific embodiments herein when AR is a substituted phenyl group, when R₁ and R₂ are both OH, R₃ is not an unsubstituted ethyl, n-butyl or benzyl. In additional specific embodiments, when AR is a substituted phenyl group and R₁ and R₂ are both methoxy groups, R₃ is not H, methyl, ethyl, n-propyl, isopropyl or benzyl. In more specific embodiments of compounds herein when AR is a substituted phenyl group and R₃ is H and R₁ is NH₂ or AcNH then R₂ is not Cl, Br, OCH₃ or NH₂. In yet more specific embodiments of compounds herein when AR is a substituted phenyl group and R₃ is H and R₁ is methoxy, then R₂ is not Cl, Br or NH₂.

In specific embodiments, the compounds of this invention have only two aromatic rings. In other embodiments, the compounds of this invention have only three aromatic rings.

In a specific embodiment, the invention relates to compounds of formula II 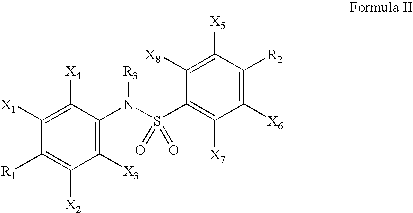
where all variables are as defined above. In specific embodiments, X₁, X₃, X₅, and X₇ are all H. In other embodiments, all of X₁, X₃, X₅, and X₇ are hydrogens and one or X₄ or X₈ are hydrogens. In specific embodiments, X₄ is linked to R₃ to form a 5-, 6- or 7-member ring as described above. In specific embodiments, X₈ is linked to R₃ to form a 5-, 6- or 7-member ring as described above.

In other specific embodiments, the invention relates to compounds of formula III: 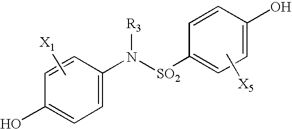
or formula IV: 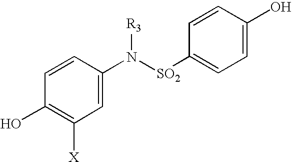
where R₃ is as defined for formula I, and X, X₁ and X₅, independently, are selected from the group consisting of halogens, optionally substituted C1-6 alkyl groups, optionally substituted C2-C6 alkenyl groups, optionally substituted C1-C6 alkoxy groups, optionally substituted —CO—R groups, optionally substituted —SR groups, cyano groups, nitro groups, thiol groups, and hydroxy groups, where R is H, or a C1-C6 alkyl group where optional substitution means substitution with one or more halogens, cyano groups, nitro groups, thiol groups, thioalkoxy groups, aryloxy, hydroxy groups, or alkoxide groups, where X₁ and X₅ represent the presence of one substituent on each ring at any ring carbon to which a bond can be formed and wherein X₁ and R₃ optionally together form a 5-, 6- or 7-member ring that can be aromatic, that may contain one or two double bonds and that in which one or two carbon atoms can be replaced with heteroatoms. (e.g., N, O or S).

In specific embodiments, the invention relates to compounds of any of formulas II-IV wherein R₃ is a fully or partially halogenated C1-C6 alkyl group or a fully or partially halogenated C2-C6 alkenyl group and to those compounds in which R3 is a fully or partially fluorinated C1-C6 alkyl group or a fully or partially fluorinated C2-C6 alkenyl group.

In specific embodiments, R₃ in any of the above formulas can be a group having the structure: 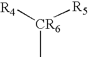
where R₄ is an optionally substituted C1-C4 alkyl group, R₅ is an optionally substituted C1-C5 alkyl group and R₆ is hydrogen or an optionally substituted methyl group. More specifically, R₆ is hydrogen, R₅ is a trifluoromethyl group and R₄ is a C1-C5 alkyl group, including a methyl group. In this embodiment X1 (or other ring substituent ) together with R₄ optionally together form a 5-, 6- or 7-member ring that can be aromatic, that may contain one or two double bonds and that in which one or two carbon atoms can be replaced with heteroatoms. (e.g., N, O or S).

In other specific embodiments, R₃ in any of the above formulas is a C1-C6 fluorinated alkyl group, particularly a C1-C3 fluorinated alkyl group and more particularly a —CH2-CH2-CF3 group.

In other specific embodiments, R₃ is an optionally substituted C1-C6 branched alkyl group, particularly a halogenated alkyl group or an unsubstituted alkyl group and more particularly a fluorinated alkyl group.

In other specific embodiments, R₃ is an optionally substituted C3-C6 cyclic alkyl group, particularly a halogenated cyclic alkyl group or an unsubstituted cyclic alkyl group and more particularly a fluorinated cyclic alkyl group. R₃ can for example be a cyclopropyl group, a fluorinated cyclopropyl group, a cyclobutyl group, a fluorinated cyclobutyl group, a cyclopentyl group, a fluorinated cyclopentyl group, a cyclohexyl group or a fluorinated cyclohexyl group. R₃ groups can also be cyclohexyl groups substituted with one or more C1-C6 alkyl groups, one or more C1-C6 alkoxide groups, one or more hydroxide groups, one or more cyano groups and/or one or more nitro groups.

The invention provides a method for selectively modulating the expression of one or more genes in a cell or tissue wherein the expression of the one or more genes is regulated by ERβ by contacting the cell or tissue with an effective amount of one or more compounds or pharmaceutically acceptable salts or prodrugs thereof of this invention. In a specific embodiment of the method, the compound, salt or prodrug exhibits a ratio of relative binding affinities (RBAs) (ERbeta/ERalpha) of 5 or more. In a preferred embodiment of the method, the compound, salt or prodrug exhibits a ratio of RBAs (ERbeta/ERalpha) of 10 or more. In a more preferred embodiment of the method, the compound, salt or prodrug exhibits a ratio of RBAs (ERbeta/ERalpha) of about 25 or more. In other specific embodiments of the method, the compound is a compound of formula IV where R is an optionally substituted C1-C6 alkyl or C2-C6 alkenyl group, particularly halogenated C1-C6 alkyl or halogenated C2-C6 alkenyl groups, and more particularly fluorinated C1-C6 alkyl groups or fluorinated C1-C6 alkenyl groups. Preferred compounds, salts and prodrugs for use in this method are those where X is H or F.

The invention further provides a method for selectively modulating the expression of one or more genes in a cell or tissue wherein the expression of the one or more genes is regulated by ERbeta by contacting the cell or tissue with an effective amount of one or more compounds or pharmaceutically acceptable salts or prodrugs thereof or stereoisomers thereof of this invention and wherein the compound, salt, stereoisomer, or prodrug thereof at the amount employed exhibits minimal effect on the expression of a gene in the cell or tissue the expression of which is regulated by ER alpha. In a preferred embodiment of the method, the compound, salt or prodrug exhibits a ratio of RBAs (ER beta/ER alpha) of 10 or more. In a more preferred embodiment of the method, the compound, salt, stereoisomer or prodrug exhibits a ratio of RBAs (ER beta/ER alpha) of about 100 or more. In other specific embodiments of the method, the compound is a compound of formula IV where R is an optionally substituted C1-C6 alkyl or C2-C6 alkenyl group, particularly halogenated C1-C6 alkyl or halogenated C2-C6 alkenyl groups, and more particularly fluorinated C1-C6 alkyl groups or fluorinated C1-C6 alkenyl groups. Preferred compounds, salts, stereoisomers and prodrugs for use in this method are those where X is H or F.

The term “alkyl” generally refers to straight-chain, branched or cyclic alkyl groups, which are monovalent. C1-C6 alkyl groups are those that contain 1 to 6 carbon atoms and include all isomeric structures. Exemplary alkyl groups are methyl, ethyl, propyl, isopropyl, butyl, isobutyl, pentyl, isopentyl, and hexyl and structural isomers thereof. Cycloalkyl groups are a subset of alkyl groups which contain a ring of carbon atoms, exemplary cycloalkyl groups are cyclopropyl, cyclobutyl, cyclopenty, and cyclohexyl groups. Unless indicated otherwise, alkyl groups are optionally substituted with one or more non-hydrogen substituents which include, among others, halogens (fluorine, chlorine, bromine and iodine), cyano groups, nitro groups, hydroxyl groups, thiol groups, thialkoxy groups (particularly C1-C6 thioalkoxy groups), alkyl groups (particularly C1-C6 alkyl groups), alkoxy groups (particularly C1-C6 alkoxyl groups), aryloxy groups, amine (or amino) groups (—NH₂), amine groups (—NHR or N(R)₂, where each R independently is an alkyl group, particularly a C1-C6 alkyl group), ether groups (e.g., —(CH₂)ₙ—[O(CH₂)ₘ—]ₚCH₂-M groups, where M is H, OH, SH, NH₂, amine or an alkyl group), —COOR groups (where R is H or alkyl). Unsubstituted alkyl groups are those alkyl groups that contain only carbon and hydrogen. Alkyl groups may be substituted with cycloalkyl groups and cycloalkyl groups may be substituted with alkyl groups. Specific subsets of alkyl groups for all variable definitions herein are unsubstituted alkyl groups, C1-C6 alkyl groups, C1-C3 alkyl groups, C8-C20 alkyl groups, and C12-C18 alkyl groups

The term “alkenyl” generally refers to a straight-chain, branched, or cyclic hydrocarbons having one or more carbon-carbon double bonds. The group is typically monovalent. The term includes monoolefins and dienes and groups containing two or more conjugated double bonds. C2-C6 alkenyl groups are those that contain 2 to 6 carbon atoms. Exemplary alkenyl groups are ethylene, propylene, isopropylene, butylene, pentylene, hexylene and various structural isomers thereof. Cycloalkenyl groups are cyclic hydrocarbons that contain one or more double bonds, such as cyclohexylene. Unless otherwise indicated alkenyl groups are optionally substituted with one or more non-hydrogen substituents such as those listed above for alkyl groups. Specific subsets of alkenyl groups for all variable definitions herein are unsubstituted alkenyl groups, mono-ene alkenyl groups, dienyl groups, C2-C6 alkenyl groups, C2-C3 alkenyl groups, C8-C20 alkenyl groups, and C12-C18 alkenyl groups.

The term “alkynyl” refers to a monoradical of an unsaturated hydrocarbon having one or more triple bonds (C≡C). Unless otherwise indicated preferred alkyl groups have 1 to 30 carbon atoms and more preferred are those that contain 1-22 carbon atoms. Alkynyl groups include ethynyl, propargyl, and the like. Short alkynyl groups are those having 2 to 6 carbon atoms, including all isomers thereof. Long alkynyl groups are those having 8-22 carbon atoms and preferably those having 12-22 carbon atoms as well as those having 12-20 carbon atoms and those having 16-18 carbon atoms. Unless otherwise indicated alkynyl groups are optionally substituted with one or more non-hydrogen substituents such as those listed above for alkyl groups. Specific subsets of alkynyl groups for all variable definitions herein are unsubstituted alkynyl groups, mono-yne alkynyl groups (containing one triple bond), diynyl groups (containing two triple bonds), C2-C6 alkynyl groups, C2-C3 alkynyl groups, C8-C20 alkynyl groups, and C12-C18 alkynyl groups.

The term “aryl” generally refers to a group containing a cyclic, aromatic hydrocarbon. Examples of aryl groups include phenyl, naphthyl, and biphenyl. Aryl generically includes heteroaryl. The term heteroaryl refers to a cyclic, aromatic hydrocarbon in which one or more of the ring carbons are replaced with a heteroatom (e.g., N, O or S). Examples of heteroaryl groups are thienyl, furyl, pyridyl, and pyrimidyl groups. Unless otherwise indicated, aryl and heteroaryl groups can be substituted with one or more non-hydrogen atoms or functional groups, such as those listed above for alkyl group. Specific subsets of aryl groups for all variable definitions herein are unsubstituted aryl groups, aryl groups substituted with one or more alkyl groups, aryl groups having one or more six-membered rings, aryl groups have one five-membered ring or one five-membered ring and one or more six-membered rings; aryl groups having 6-11 C atoms, aryl groups having 12-24 C atoms and heteroaryl groups which belong to any one of the specifically listed subsets above.

The terms “alkoxy” and thioalkoxy” refer respectively to groups of formula —O—R and —S—R, where R is an alkyl group that is optionally substituted as noted above for alkyl groups. Alkoxy and thioalkoxy groups include those having from 1-30 carbon atoms, and more particularly include those that have C1-C6 alkyl groups. Alkoxy and thioalkoxy groups include those having C1-C3 alkyl groups, C8-C20 alkyl groups, and C12-C18 alkyl groups. The term “aryloxy” refers to a groups of formula —OR where R is an aryl group as defined above including an optionally substituted aryl group.

The term “amino” or “amine” refers to the group —NH₂ or to the group —N(R₁₀)₂ where each R₁₀ is independently selected from the group consisting of hydrogen, alkyl, cycloalkyl, alkenyl, cycloalkenyl, alkynyl, aryl, heteroaryl and heterocyclic provided that both R₁₀ are not hydrogen. As noted above the various alkyl, alkenyl, alkynyl and aryl groups of any amine group are optionally substituted as discussed above.

“Haloalkyl” refers to alkyl as defined herein substituted by one or more halides (e.g., F—, Cl—, I—, Br—) as defined herein, which may be the same or different. A haloalkyl group may, for example, contain 1-10 halide substituents. Representative haloalkyl groups include, by way of example, trifluoromethyl, 3-fluorododecyl, 12,12,12-trifluorododecyl, 2-bromooctyl, 3-bromo-6-chloroheptyl, and the like. Haloalkyl groups include fluoroalkyl groups. The terms “haloalkenyl”, haloalkynyl” and “haloaryl” have analogous meaning herein.

The term “sulfonamide” refers to the group: 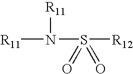
wherein most generally, each R₁₁ and R₁₂ are independently selected from the group consisting of hydrogen, alkyl, cycloalkyl, alkenyl, cycloalkenyl, alkynyl, aryl, heteroaryl and heterocyclic provided that R₁₂ is not hydrogen. As noted above the alkyl, alkenyl, alkynyl and aryl groups of any amine group are optionally substituted. As discussed above.

Other chemical terms used herein which are not specifically defined are intended to have the broadest meaning that the terms have in the art that is consistent with the context of their use herein.

The term “mammal” is intended to take its usual biological meaning and refers to animals including, for example, dogs, cats, cows, sheep, horses, and humans. Preferred mammals include humans.

The term “pharmaceutically acceptable” used in reference to a compound, composition or salt indicates that the designated species is appropriate for use for administration to an individual. The pharmaceutically acceptable species of this invention are particularly useful for administration to mammals, including dogs, cats, horses, cows, sheep and humans and are intended to be suitable for use in one or more of such mammals. Preferred mammals are humans and pharmaceutically acceptable species herein are intended to be appropriate for administration to humans.

The term “prodrug” is used generally herein as broadly as the term is used in the art and refers to a compound that is a drug precursor which, following administration, releases the drug in vivo via a chemical or physiological process (for example, by a change in pH or through enzyme activity). A discussion of the use of prodrugs is provided by T. Higuchi and W. Stella, “Prodrugs as Novel Delivery Systems, Vol. 14 of the ACS Symposium Series, and in Bioreversible Carriers in Drug Design, ed. Edward B. Roche, American Pharmaceutical Association and Pergamon Press, 1987.

The term “salt” refers to organic and inorganic salts of any compound stereoisomer, or prodrug of this invention. Salts can be prepared as is known in the art employing a suitable organic or inorganic acid or base and isolating the salt thus formed. Representative salts include the hydrobromide, hydrochloride, sulfate, bisulfate, nitrate, acetate, oxalate, besylate, palmitate, stearate, laurate, borate, benzoate, lactate, phosphate, tosylate, citrate, maleate, fumarate, succinate, tartrate, naphthylate, mesylate, glucoheptonate, lactobionate, and laurylsulphonate salts, among others. These may also include cations based on the alkali and alkaline earth metals, such as sodium, lithium, potassium, calcium, magnesium, and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like.

Sulfonamides of this invention are prepared by methods described herein or by routine adaptation of those methods by routine variation in starting material, solvent, temperature or pressure, and/or reagent. Those of ordinary skill in the art can prepare the sulfonamides of this invention in view of the description herein, particularly in the examples, and in further view of methods, techniques and reagents that are well known in the art. Additional representative methods for preparation of sulfonamides are provided in references cited herein which are, at least in part, incorporated by reference herein to provide a description of such methods.

Synthetic methods that can be employed for the synthesis of compounds of this invention are discussed in more detail hereafter. One of ordinary skill in the art can prepare the compounds of this invention in view of the specific teachings herein and in further view of what is well known in the art concerning methods of synthesis of organic compounds.

The sulfonamide system is amenable to preparation of multiple analogs quickly. Sulfonamide 7 was prepared according the procedure published by Stauffer et al. 2000 and was alkylated with primary and secondary alkyl halides 8a-j to yield sulfonamides 9a-j of various sizes and hydrophobic character (Scheme 1).

Several methods were attempted to generate sulfonamides with N-halogenated alkyl groups, particularly a CF₃ group. Efforts to alkylate compound 7 (Scheme 1) with alkyl iodides 10a-b (Scheme 2) were unsuccessful. This was likely due to the inductive effects of the CF₃ group which can stabilize a partially positive carbon α or β to the CF₃ group. In addition, the electronegative fluorines are likely to repel the sulfonamide anion, the approaching nucleophile. An alternative route based on the reaction of an amine with trifluoroacetic anhydride (TFAA) was successful. As shown in Scheme 3, p-anisidine 11 was reacted with TFAA 12 to yield the trifluoromethyl acetamide 13. The carbonyl of 13 was reduced with borane to yield the 2,2,2-trifluoroethyl substituted aniline 14.

A slightly different procedure was used for the incorporation of the 3,3,3-trifluoropropyl group, which employed the commercially available carboxylic acid. Using DCC, 11 was coupled to 15 to yield amide 16, which was reduced to yield the desired product 17. To prepare the sulfonamides 19a-b, 4-methoxybenzene-sulfonylchloride 18 was reacted with 14 and 17. The 2-methyl protected sulfonamides were demethylated using boron tribromide (Scheme 4) to generate free hydroxyls.

To add halogens to the phenyl rings of sulfonamides, anisidines 21a-b were selected as the starting materials. Scheme 5 shows the synthesis of the N-propyl sulfonamides containing a fluorine or chlorine attached to the aniline ring. Compounds 21a-b can be reacted with 18 to yield the unalkylated sulfonamides 22a-b; these were then reacted with 8c to give 23a-b. Compounds 23a-b were demethylated with boron tribromide to 24a-b.

The synthesis of the 3,3,3-trifluoropropylsulfonamides couples compounds 21a-b and 15 using DCC to yield amides 25a-b (Scheme 6), which are reduced with borane to yield amines 26a-b. Amines 26a-b were reacted with 18 to yield the protected sulfonamides 27a-b. Demethylation using boron tribromide provides sulfonamides 28a-b.

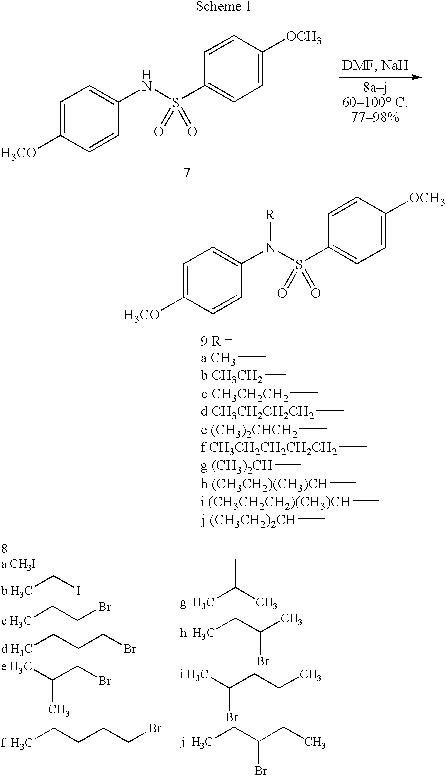

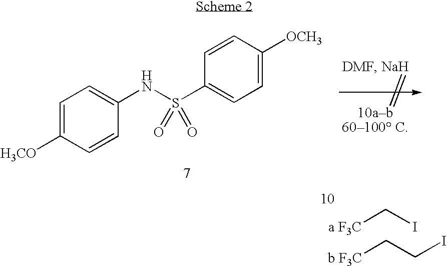

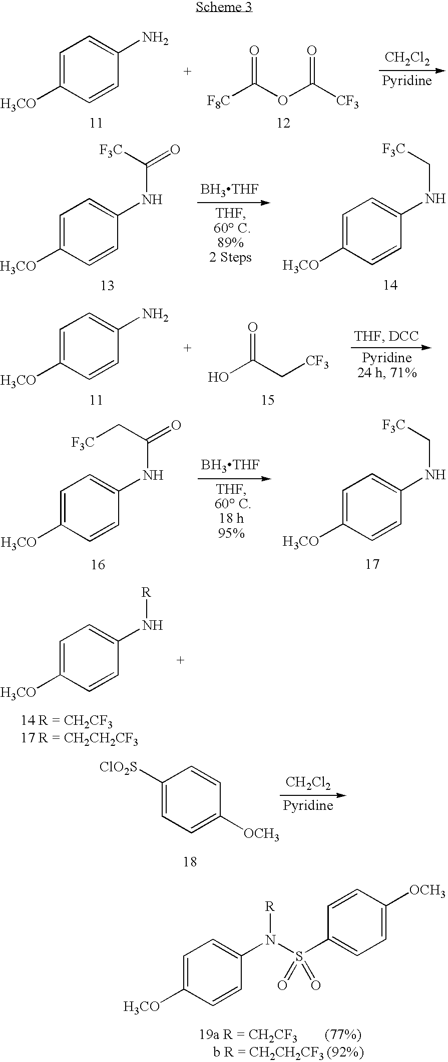

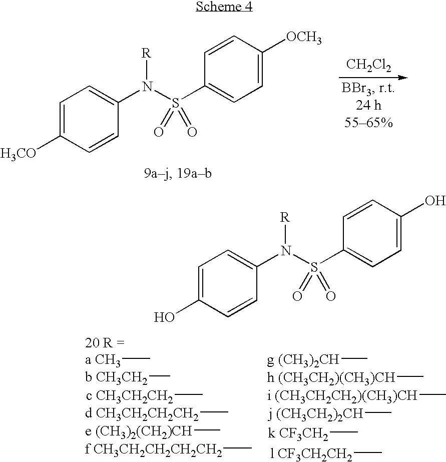

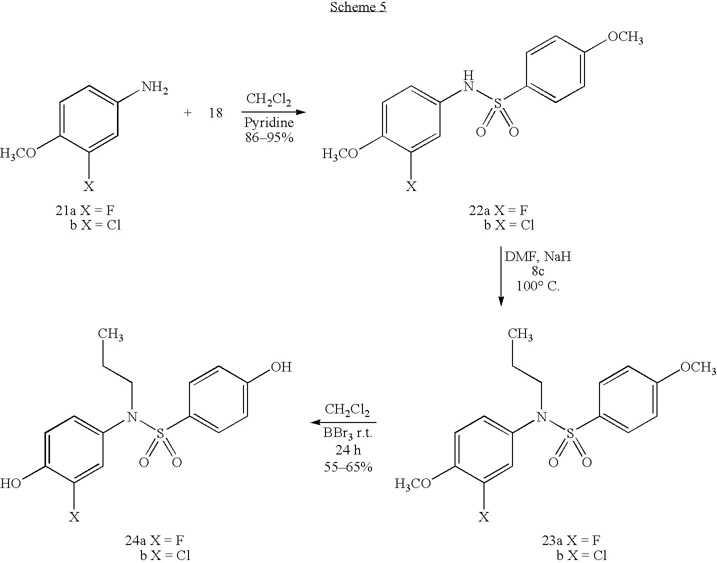

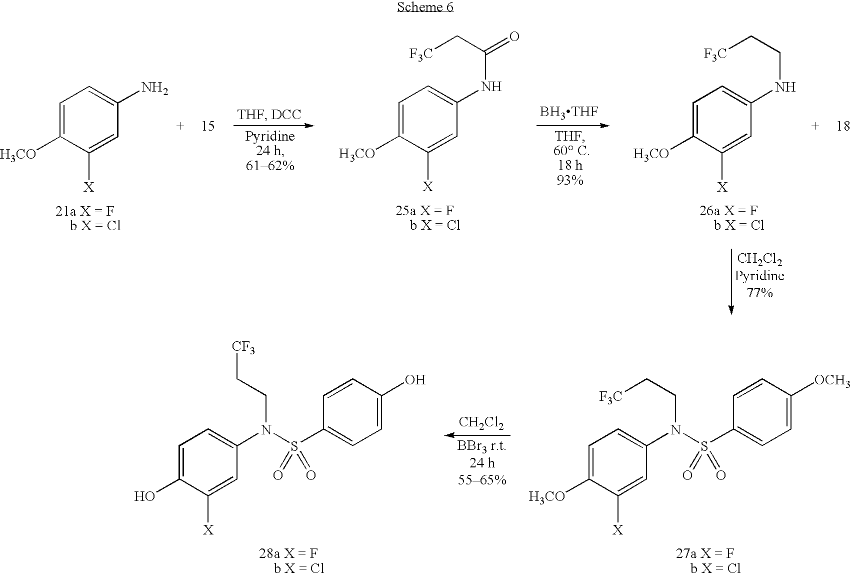

The relative binding affinities (RBAs) of potential ER ligands can be measured using purified full length human ERalpha and ERbeta receptors in a competitive radiometric binding assay, according to published procedures (Carlson, K. E.; Choi, I.; Gee, A,; Katzenellenbogen, B. S.; Katzenellenbogen, J. A. Altered ligand binding properties and enhanced stability of a constitutively active estrogen receptor: evidence that an open pocket conformation is required for ligand interaction. Biochemistry 1997, 36, 14897-14905; Katzenellenbogen, J. A.; Johnson, H. J., Jr.; Myers, H. N. Photoaffinity labels for estrogen binding proteins of rat uterus. Biochemistry 1973, 12, 4085-4092) as described in the Examples.

The RBA values of certain compounds of formula (IV):

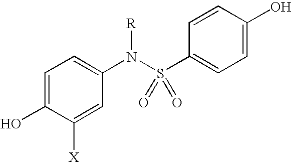

are listed in Table 1, and are normalized to estradiol, which is set at 100%.

TABLE 1
Relative Binding affinities (RBAs) of N-alkylsulfonamides
for ERalpha and ERbeta¹

| X | Cmpd No. | R | ERα | ERβ | ERβ/ERα |
|---|---|---|---|---|---|
| H | 20a | methyl | 0.009 | 0.058 | 6.4 |
| H | 20b | ethyl | 0.007 | 0.28 | 40 |
| H | 20c | n-propyl (FS-2) | 0.026 | 2.56 | 98 |
| H | 20d | n-butyl | 0.023 | 1.15 | 50 |
| | 20f | n-pentyl | 0.047 | 0.60 | 13 |
| | 20m | n-hexyl | 0.056 | 0.44 | 7.8 |
| | 20g | isopropyl | 0.012 | 0.22 | 18 |
| | 20e | —CH₂CH(CH₃)₂ | 0.013 | 0.14 | 11 |
| | 20h | —CH(CH₃)(CH₂CH₃) | 0.006 | 0.97 | 162 |
| | | (Racemic) | | | |
| | 20h | —CH(CH₃)(CH₂CH₃) | 0.019 | 1.43 | 75 |
| | | [R enantiomer] | | | |
| | 20h | —CH(CH₃)(CH₂CH₃) | 0.004 | 0.279 | 70 |
| | | [S enantiomer] | | | |
| | 20i | —CH(CH3)(CH₂CH₂CH₃) | 0.013 | 1.3 | 100 |
| | 20j | —CH(CH₂CH₃)₂ | 0.018 | 0.11 | 6.1 |
| | 20n | —CH(CH₂)₄² | 0.016 | 0.056 | 3.5 |
| | 20k | —CH₂CF₃ | 0.020 | 0.17 | 8.5 |
| | 20l | —CH₂CH₂CF₃ (FS-5) | 0.006 | 1.27 | 212 |
| | 24a | —CH₂CH₂CH₃ | 0.011 | 0.254 | 23 |
| | 28a | —CH₂CH₂CF₃ | 0.009 | 0.178 | 20 |
| | 24b | —CH₂CH₂CH₃ | 0.005 | 0.038 | 7.6 |
| | 28b | —CH₂CH₂CF₃ | 0.003 | 0.023 | 7.7 |

¹See Schemes 4-6 for compound numbers;
²—CH(CH₂)₄ is the cyclopentyl group.

In general the compounds in Table 1 exhibit low binding affinities for ER alpha. Compounds 20f and 20m have the highest ERalpha affinities of 0.047% and 0.056%, respectively. The entire series of compounds is ER beta selective, even when there is low ER beta affinity. The ERbeta binding affinities range from modest to good (0.1 to about 3%). Compound 20c has the highest ER beta affinity of 2.56%, and it is 98-fold selective for ER beta. Although 20c has good selectivity, compounds 20h (racemic) and 20 l have the greatest ER beta selectivity (162- and 212-fold, respectively). Fluorination of the alkyl group of the N-alkyl sulfonamides tended to increase ER beta selectivity, but in general caused a decrease in ER beta binding affinity relative to estradiol.

In specific embodiments herein, ER ligands exhibit ER beta binding affinities of about 0.1% or more. In other embodiments herein, ER ligands exhibit ER beta binding affinities of about 0.2% or more. In other embodiments herein, ER ligands exhibit ER beta binding affinities of about 0.5% or more. In other embodiments herein, ER ligands exhibit ER beta binding affinities of 1% or more. In other embodiments herein, ER ligands exhibit ER beta binding affinities of 2% or more.

In specific embodiments herein, ER ligands exhibit ER beta/ERalpha binding selectivity of 2 or more. In other embodiments herein, ER ligands exhibit ER beta/ER alpha binding selectivity of 5 or more. In additional embodiments herein, ER ligands exhibit ER beta/ER alpha binding selectivity of about 10 or more. In yet other embodiments herein, ER ligands exhibit ER beta/ER alpha binding selectivity of about 20 or more. In more preferred embodiments, ER ligands exhibit ER beta/ER alpha binding selectivity of about 50 or more or about 100 or more

In addition, it has been noted that N-alkyl sulfonamides are stable without loss of activity with respect to ER when stored at −20 C for an extended period of time.

The agonist or antagonist character of potential ER ligands as regulators of transcription can be assessed in cells transiently transfected with ERalpha or ERbeta, for example, by co-transfection assays in human endometrial cells (HEC-1), using expression plasmids for either ER alpha or ER beta, and an estrogen-responsive reporter gene as described in the experimental section (See also: McInerney, E. M.; Tsai, M. J.; O'Malley, B. W.; Katzenellenbogen, B. S. Analysis of estrogen receptor transcriptional enhancement by a nuclear hormone receptor coactivator. Proc. Natl. Acad. Sci. USA 1996, 93, 10069-10073). The agonist or antagonist character of potential ER ligands as regulators of transcription can alternatively be assessed in other cell-types and employing different promoters and reporter genes. Furthermore, the agonist and antagonist character of potential ER ligands can be assessed employing either reporter gene expression or endogenous gene expression in cells which stably express ERalpha and/or ERbeta, for example, in U2-OS cells expressing either ERalpha or ER beta. The results of such assessments of ER ligands in different cells, employing different promoters or reporter genes and employing either reporter gene expression or endogenous gene expression may quantitatively differ. As will be appreciated in the art, specific comparisons of the agonist or antagonist character of ER ligands are best made employing the same methods.

The agonist or antagonist character of potential ER ligands as regulators of transcription can be assessed in animal model systems in which the affect of the ligand on various tissues, e.g., uterus, pituitary, liver, bone or brain; on body weight; uterus weight; plasma cholesterol; gene expression (e.g., complement C3 gene expression); induction of progesterone receptor mRNA in the brain; or in hot flush prevention; among a number of other in vivo effects is measured. In vivo assays of ER ligands are described for example in Harris et al. (2002) “ER alpha-Mediated In Vivo Responses,” Endocrinology 143(11):4172-4177; Hillisch et al. (2004) “Dissecting Physiological Roles of Estrogen Receptor Alpha and Beta with Potential Selective Ligands from Structure-Based Design,” Molecular Endocrinology 18(7):1599-1609; and Merchenthaler et al. (1998) “The Effect of Estrogens and Antiestrogens in a Rat Model for Hot Flush,” Maturitas 30:307-316.

As used herein and as understood in the art potency refers to the dose required to get an effect, generally expressed as EC50. In contrast, efficacy refers to the maximum level of effect observed at a high dose and is used to characterize compounds as agonists, antagonsits, or mixed agonist/antagonists.

Without wishing to be bound by any particular theory, it is believed that the smaller overall size of the sulfonamide ligands prevents them from binding well to ER alpha and allows for good ER beta affinity selectivity.

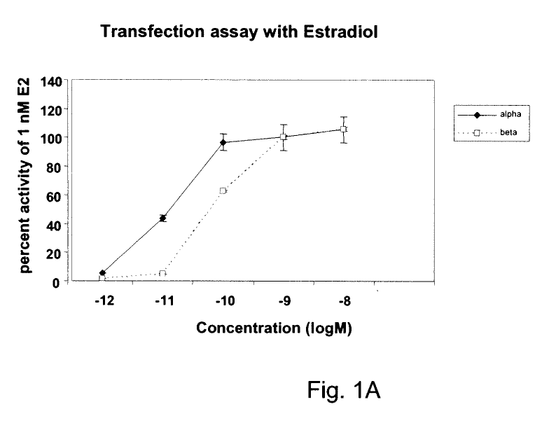

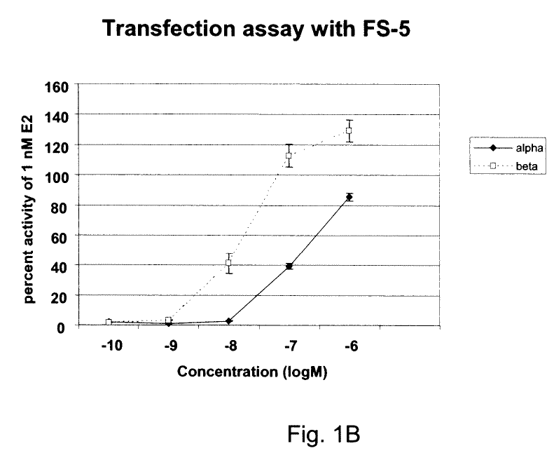

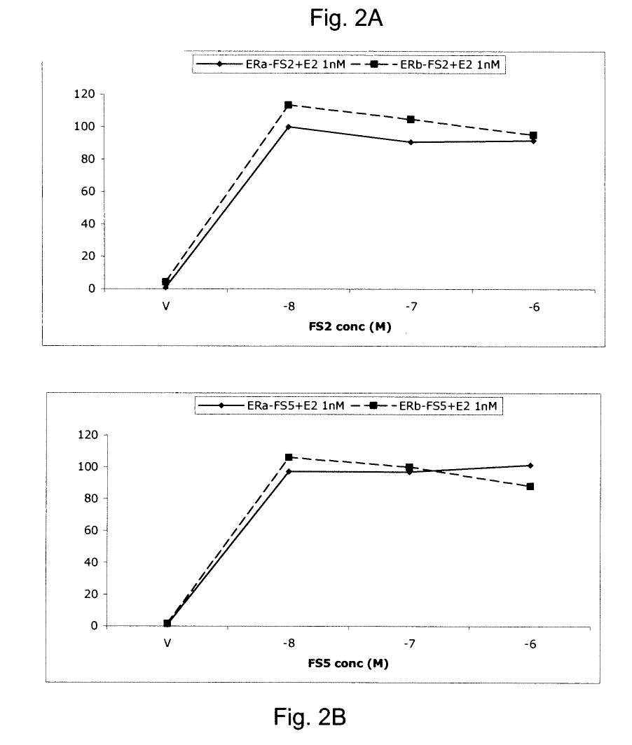

Dose-response curves for estradiol and sulfonamide FS-2 (R is n-propyl, 20b) in HEC-1 cells transiently transfected with ERalpha or ERbeta are shown in FIGS. 1A-B where the responses on ERalpha (solid line) and ERbeta (dashed line) are compared. Data in FIGS. 1A-B were obtained employing constructs containing the pS2 promoter. As shown in FIGS. 2A and 2B, ERbeta-selective ligands of this invention exhibit little or no measurable antagonism on estrogen-induced gene expression. The data presented was measured using a reporter gene under the control of the pS2 promoter. Thus, activation of transcription by estradiol through either ERalpha or ERbeta is expected to be unaffected by ER beta ligands of this invention, particularly by FS-2 and FS-5. These ligands do not act as antagonists of estrogen action through either ERalpha or ER beta.

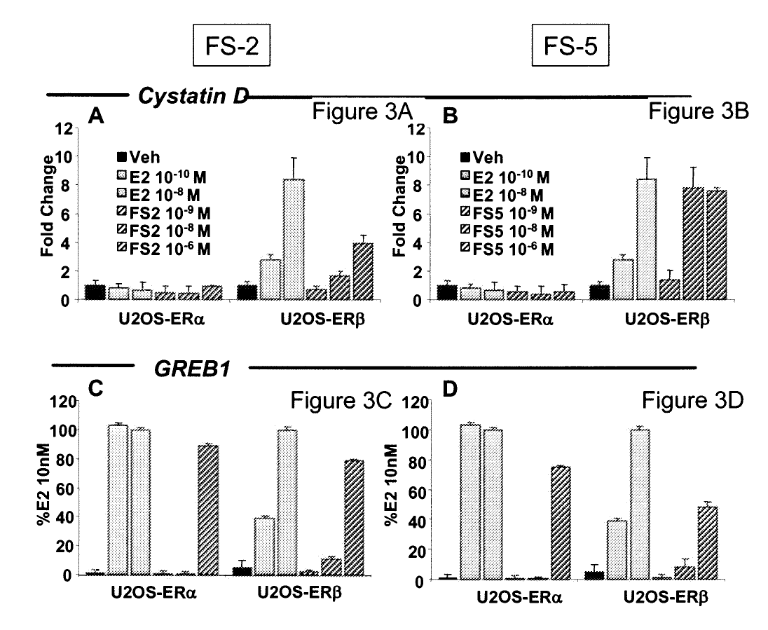

As shown in FIGS. 3A-D, the regulation of endogenous genes (e.g., cystatin D and GREB1), by ERbeta-selective ligands of this invention, particularly FS-2 and FS-5, exhibits preferential regulation through ERbeta. Cystatin D is a gene that is activated only through ERbeta, whether by estradiol, or the ligands of this invention. GREB1 is activated by estradiol through both ER alpha and ER beta. Compared to the dose response of estradiol, FS-2 and FS-5, which are representative ligands of this invention, show preferential potency through ERbeta.

The non-steroidal subtype-selective ER ligands of this invention are particularly useful in pharmaceutical applications for prevention or treatment of estrogen-responsive disorders and conditions, as active ingredients of pharmaceutical compositions in combination with a pharmaceutically acceptable carrier or exipient. Compounds of formulas I-IV can also exhibit selective activation of ER subtypes, variants, and/or mutants for selective regulation of ER-responsive genes. The ER ligands may be combined with each other to achieve a desired pharmaceutical response or administered in combination with known estrogens, progestin, or antiestrogens. The ER ligand is present in the pharmaceutical compositions in an amount, or in combination with other ligands in a combined amount, sufficient to selectively induce or inhibit a desired estrogen response. In those cases in which the ER ligand selectively interacts with an ER subtype variant or mutant, the amount of ligand (or combined amount of ligands) present in the pharmaceutical composition is in the range that induces or inhibits the desired selective response. The invention also relates to methods of preventing or treating estrogen-responsive disorders and physiological conditions employing pharmaceutical compositions comprising ER ligands of this invention alone or in combination.

Pharmaceutical compositions of this invention can also include other steroid or non-steroid ER ligands which may supplement or enhance the activity of the composition for a particular medical application. Pharmaceutical compositions of this invention include those which are useful in the prevention or treatment of hormone-dependent cancers, including breast cancer, those useful for hormone-replacement therapy, those useful in the treatment of infertility, those useful for prevention or treatment of osteoporosis, those useful for providing cardiovascular, CNS (suppress hot flashes, provide cognitive improvements, etc.) or related benefits, and those useful for lowering serum cholesterol levels.

ER ligands of this invention can exhibit agonist or antagonist behavior in vitro, in vivo and ex vivo which is selective or specific for a given ER subtype, variant or mutant(e.g., ER beta). In general, ER ligands can be selective in potency (i.e., a more potent agonist for ERbeta than for ER alpha, or in character (an agonist on ER beta and an antagonist on ER alpha). These functions can be assessed for a given ER ligand or ligand mixture employing in vitro, in vivo and/or ex vivo methods known in the art or as described in the Examples herein. A number of ER alpha agonists are known in the art. A number of ER alpha selective antagonists are known in the art. Examples of ER alpha selective antagonists are found in Sun J, Huang Y R, Harrington W R, Sheng S, Katzenellenbogen J A, Katzenellenbogen B S. Antagonists selective for estrogen receptor alpha. Endocrinology. March 2002; 143(3):941-7 and Stauffer S R, Huang Y R, Aron Z D, Coletta C J, Sun J, Katzenellenbogen B S, Katzenellenbogen J A. Triarylpyrazoles with basic side chains: development of pyrazole-based estrogen receptor antagonists. Bioorg Med Chem. January 2001; 9(1):151-61.

Pharmaceutical compositions of this invention can be provided in a variety of dosage forms including without limitation pills for oral administration, solutions or emulsions for oral administration or for injection.

ER ligands are useful in vitro, in vivo and/or ex vivo for selective activation or repression of expression, dependent upon the agonist or antagonist nature of the ligand, of a gene regulated by an ER (estrogen receptor). Gene activation or repression can be selective with respect to subtype of ER (e.g., ER alpha or ER beta), variant of ER (e.g., splice variant forms, truncated or processed forms, covalently modified forms, etc.) or mutant of ER. The term “in vitro” is intended to have its broadest art-recognized meaning and generally refers to an artificial environment and to processes or reactions that occur within an artificial environment. In vitro environments can consist of, but are not limited to, test tubes and cell cultures. Similarly, the term “in vivo” is intended to have its broadest art-recognized meaning and in general refers to the natural environment (e.g., an animal or a cell) and to processes or reaction that occur within a natural environment. The ER ligands of this invention are also useful for selective activation or repression of expression as noted above of a gene regulated by an ER in ex vivo systems, where the term “ex vivo” is also intended to have its broadest art-recognized meaning and generally refers to cells or tissue that are obtained from a natural environment to be manipulated typically with the intention that the manipulated cells are to returned to a natural environment (not necessarily the environment from which the cells or tissue were obtained).

ER ligands are also useful in vitro, in vivo and ex vivo for selective regulation of cellular activities under the control of ER. Cellular activities may be regulated in a variety of ways by ER, subtypes of ER or variants of ER, e.g., up or down regulation of a given cellular process. Regulation is selective with respect to subtype of ER (e.g., ER alpha or ER beta), or variant of ER (e.g., splice variant forms, truncated or processed forms, covalently modified forms, etc.). Cellular activities that may be regulated include both genomic (related to ER-responsive gene expression) or non-genomic activities (not directly related to gene expression, e.g., such as regulation of calcium flux, particularly in bone cells, hormone release, particularly prolactin release from pituitary cells, etc.).

The biological, therapeutic and pharmaceutical uses for an ER beta-selective agonist can be organized on the basis of the general biological effect of ER beta compared to the other ER subtype, ER alpha, the tissues and organs in which ER beta is predominant or present at significant concentrations together with ER alpha, the effects in these tissues or organs that have been observed as a result of the genetic deletion of ER beta,(Couse, J. F.; Korach, K. S., Estrogen receptor null mice: what have we learned and where will they lead us? Endocr Rev 1999, 20, (3), 358-417; Pettersson, K.; Gustafsson, J. A., Role of estrogen receptor beta in estrogen action. Annu Rev Physiol 2001, 63, 165-92) or, in some cases, the effect of other ER alpha or ER beta selective ligands, and various endocrine disorders that might occur in these tissues or organs that can be treated with ER beta-selective ligands (Mueller, S. O.; Korach, K. S., Estrogen receptors and endocrine diseases: lessons from estrogen receptor knockout mice. Curr Opin Pharmacol 2001, 1, (6), 613-9.)

The General Biological Activity of ER beta—Like ER alpha, ER beta functions as a transcriptional regulator, but in general its effectiveness in this role is less than that of ER alpha, with the result that estrogen agonists acting through ER beta often oppose the effect of the same compound acting through ER alpha. Because estrogen action through ER beta has a general moderating effect on the activity of ER alpha, ER beta has been said to act as a “brake” on ER alpha activity (Matthews, J.; Gustafsson, J. A., Estrogen signaling: a subtle balance between ER alpha and ER beta. Mol Interv 2003, 3, (5), 281-92), and ER alpha and ER beta are said to have a “Yin Yang” relationship(Lindberg, M. K.; Moverare, S.; Skrtic, S.; Gao, H.; Dahlman-Wright, K.; Gustafsson, J. A.; Ohlsson, C., Estrogen receptor (ER)-beta reduces ER alpha-regulated gene transcription, supporting a “ying yang” relationship between ER alpha and ER beta in mice. Mol Endocrinol 2003, 17, (2), 203-8.)

Thus, because estrogen agonist action through ER alpha frequently causes proliferation of cells in different tissues, estrogen agonist action through ER beta can have an antiproliferative effect in certain organs and cancers. Estrogen acting through ER beta also can have a variety of other positive effects, such as anti-inflammatory, cardiovascular protective, immune protective, and antidepressive, and fertility enhancing effects.

Antiproliferative effects of ER beta: Breast and Prostate Cancers—In ER beta knockout mice, there are some reports of hyperplasia in the prostate (Weihua, Z.; Warner, M.; Gustafsson, J. A., Estrogen receptor beta in the prostate. Mol Cell Endocrinol 2002, 193, (1-2), 1-5), which is often considered a precursor of prostate cancer (although there is not universal agreement on this phenomenon, see Jarred, R. A.; McPherson, S. J.; Bianco, J. J.; Couse, J. F.; Korach, K. S.; Risbridger, G. P., Prostate phenotypes in estrogen-modulated transgenic mice. Trends Endocrinol Metab 2002, 13, (4), 163-8.) This hyperplasia is thought to be the consequence of the loss in this organ of the ER beta restraint on the proliferative effects of estrogen acting through ER alpha. Thus, an ER beta-selective ligand can reduce prostate hyperplasia, as encountered in benign prostatic hyperplasia (BPH) and in prostate cancer.

Similarly, in breast cancer there appears to be a progressive loss in the levels of ER beta relative to ER alpha as the disease progresses and tumor cells become more proliferative. See: Balfe, P. J.; McCann, A. H.; Welch, H. M.; Kerin, M. J., Estrogen receptor beta and breast cancer. Eur J Surg Oncol 2004, 30, (10), 1043-50; Hayashi, S. I.; Eguchi, H.; Tanimoto, K.; Yoshida, T.; Omoto, Y.; Inoue, A.; Yoshida, N.; Yamaguchi, Y., The expression and function of estrogen receptor alpha and beta in human breast cancer and its clinical application. Endocr Relat Cancer 2003, 10, (2), 193-202; Pearce, S. T.; Jordan, V. C., The biological role of estrogen receptors alpha and beta in cancer. Crit Rev Oncol Hematol 2004, 50, (1), 3-22; Bardin, A.; Boulle, N.; Lazennec, G.; Vignon, F.; Pujol, P., Loss of ER beta expression as a common step in estrogen-dependent tumor progression. Endocr Relat Cancer 2004, 11, (3), 537-51; Esslimani-Sahla, M.; Simony-Lafontaine, J.; Kramar, A.; Lavaill, R.; Mollevi, C.; Warner, M.; Gustafsson, J. A.; Rochefort, H., Estrogen receptor beta (ER beta) level but not its ER beta cx variant helps to predict tamoxifen resistance in breast cancer. Clin Cancer Res 2004, 10, (17), 5769-76; and Roger, P.; Sahla, M. E.; Makela, S.; Gustafsson, J. A.; Baldet, P.; Rochefort, H., Decreased expression of estrogen receptor beta protein in proliferative preinvasive mammary tumors. Cancer Res 2001, 61, (6), 2537-41. Thus, the selective activation of ER beta by an ER beta ligand can suppress or reverse the progression of breast cancer.

Antiinflammatory Effects of Estrogens Acting Through ER beta—There are significant levels of ER beta in the colon (Matthews and Gustafsson 2003, supra). ER beta-selective ligands have also been effective in other models of inflammation, namely in a rheumatoid arthritis model (Harris, H. A.; Albert, L. M.; Leathurby, Y.; Malamas, M. S.; Mewshaw, R. E.; Miller, C. P.; Kharode, Y. P.; Marzolf, J.; Komm, B. S.; Winneker, R. C.; Frail, D. E.; Henderson, R. A.; Zhu, Y.; Keith, J. C., Jr., Evaluation of an estrogen receptor-beta agonist in animal models of human disease. Endocrinology 2003, 144, (10), 4241-9.) Spleens of ER beta knockout mice show proinflammatory changes, consistent with the anti-inflammatory effects shown by the ER beta-selective ligands (Zhang, Q. H.; Cao, J.; Hu, Y. Z.; Huang, Y. H.; Lu, S. Y.; Wei, G. Z.; Zhao, Y. F., Morphological observation of immunological alterations in the spleens from estrogen receptor deficient mice. Xi Bao Yu Fen Zi Mian Yi Xue Za Zhi 2004, 20, (1), 1-6.)

Effects of Estrogens Acting Through ER beta in the Cardiovascular System—There are significant levels of ER beta in vascular endothelium, and ER beta appears to play a role in regulating blood pressure. ER beta-knockout mice had a hypertension phenotype. This indicates that ER beta-selective ligands can be effective as anti-hypertensive agents (Zhu, Y.; Bian, Z.; Lu, P.; Karas, R. H.; Bao, L.; Cox, D.; Hodgin, J.; Shaul, P. W.; Thoren, P.; Smithies, O.; Gustafsson, J. A.; Mendelsohn, M. E., Abnormal vascular function and hypertension in mice deficient in estrogen receptor beta. Science 2002, 295, (5554), 505-8.)

Effects of Estrogens Acting Through ER beta in the Ovary—The ovary is one of the most ER beta-rich tissues, with this ER subtype being localized to the granulosa cells. There is a severe ovarian phenotype in ER beta knockout mice; the ovaries are hemorrhagic. An ER beta-selective ligand was found to stimulate folliculogenesis and ovulation, and can be effective in enhancing fertility (Hegele-Hartung, C.; Siebel, P.; Peters, O.; Kosemund, D.; Muller, G.; Hillisch, A.; Walter, A.; Kraetzschmar, J.; Fritzemeier, K. H., Impact of isotype-selective estrogen receptor agonists on ovarian function. Proc Natl Acad Sci U S A 2004, 101, (14), 5129-34.) As in breast cancer, there appears to be a progressive loss of ER beta in ovarian cancer, and in cell models of ovarian cancer, upregulation of ER beta reduces the malignant phenotype, suggesting that ER beta-selective ligands can be helpful in the prevention and/or management of ovarian cancer (Bardin, A.; Hoffmann, P.; Boulle, N.; Katsaros, D.; Vignon, F.; Pujol, P.; Lazennec, G., Involvement of estrogen receptor beta in ovarian carcinogenesis. Cancer Res 2004, 64, (16), 5861-9; Cunat, S.; Hoffmann, P.; Pujol, P., Estrogens and epithelial ovarian cancer. Gynecol Oncol 2004, 94, (1), 25-32.)

Effects of Estrogens Acting Through ER beta in the Uterus—Though ER alpha predominates in the uterus, there are detectable levels of ER beta in this organ. An ER beta-selective ligand has been shown to be effective in causing regression of endometriosis in an animal model of human endometriosis (Harris, H. A.; Bruner-Tran, K. L.; Zhang, X.; Osteen, K. G.; Lyttle, C. R., A selective estrogen receptor-{beta} agonist causes lesion regression in an experimentally induced model of endometriosis. Hum Reprod 2005, 20, (4), 936-41.) Because endometriosis is a common gynecological problem and the cause of infertility in women of reproductive age, ER beta-selective ligands can be useful in treatment of endometriosis.

Effects of Estrogens Acting Through ER beta in Bone—While a positive effect of ER beta-selective ligands in the bone has not been established, it is clear that bone mineral density is related to different polymorphic forms of ER beta (Shearman, A. M.; Karasik, D.; Gruenthal, K. M.; Demissie, S.; Cupples, L. A.; Housman, D. E.; Kiel, D. P., Estrogen receptor beta polymorphisms are associated with bone mass in women and men: the Framingham Study. J Bone Miner Res 2004, 19, (5), 773-81.) Also, in bone cell models, ER alpha and ER beta have been shown to regulate distinct sets of gene (Stossi, F.; Barnett, D. H.; Frasor, J.; Komm, B.; Lyttle, C. R.; Katzenellenbogen, B. S., Transcriptional profiling of estrogen-regulated gene expression via estrogen receptor ER alpha or ER beta in human osteosarcoma cells: distinct and common target genes for these receptors. Endocrinology 2004, 145, (7), 3473-86; Monroe, D. G.; Getz, B. J.; Johnsen, S. A.; Riggs, B. L.; Khosla, S.; Spelsberg, T. C., Estrogen receptor isoform-specific regulation of endogenous gene expression in human osteoblastic cell lines expressing either ER alpha or ER beta. J Cell Biochem 2003, 90, (2), 315-26) and studies in ER beta knockout mice suggests that ER beta promotes the closure of bone growth plates (Chagin, A. S.; Lindberg, M. K.; Andersson, N.; Moverare, S.; Gustafsson, J. A.; Savendahl, L.; Ohlsson, C., Estrogen receptor-beta inhibits skeletal growth and has the capacity to mediate growth plate fusion in female mice. J Bone Miner Res 2004, 19, (1), 72-7.) Thus, ER beta-selective ligands can have useful effects on bone. ER beta-selective ligands can be useful in treatment of osteoporosis.

Effects of Estrogens Acting Through ER beta in the Immune System—Pathological changes in ER beta knockout mice suggest increased risk of autoimmune diseases (Zhang, Q. H.; Huang, Y. H.; Hu, Y. Z.; Wei, G. Z.; Han, X. F.; Lu, S. Y.; Zhao, Y. F., Disruption of estrogen receptor beta in mice brain results in pathological alterations resembling Alzheimer disease. Acta Pharmacol Sin 2004, 25, (4), 452-7.) ER beta knockout mice also show myeloproliferative disease resembling human chronic myeloid leukemia with lymphoid blast crisis (Shim, G. J.; Wang, L.; Andersson, S.; Nagy, N.; Kis, L. L.; Zhang, Q.; Makela, S.; Warner, M.; Gustafsson, J. A., Disruption of the estrogen receptor beta gene in mice causes myeloproliferative disease resembling chronic myeloid leukemia with lymphoid blast crisis. Proc Natl Acad Sci U S A 2003, 100, (11), 6694-9.) Thus, it is anticipated that ER beta-selective ligands can be effective in treating myeloid and lymphoid leukemia and lymphoproliferative autoimmune diseases.

Effects of Estrogens Acting Through ER beta in the Brain—ER beta is found in various regions of the brain, often together with ER alpha, but it also predominates in certain brain regions. The ER beta knockout mouse shows behavioral abnormalities, namely enhanced aggression (Nomura, M.; Durbak, L.; Chan, J.; Smithies, O.; Gustafsson, J. A.; Korach, K. S.; Pfaff, D. W.; Ogawa, S., Genotype/age interactions on aggressive behavior in gonadally intact estrogen receptor beta knockout (betaERKO) male mice. Horm Behav 2002, 41, (3), 288-96), and the accumulation in the brain of amyloid plaques characteristic of Alzheimer's disease (Zhang, Q. H.; Huang, Y. H.; Hu, Y. Z.; Wei, G. Z.; Han, X. F.; Lu, S. Y.; Zhao, Y. F., Disruption of estrogen receptor beta in mice brain results in pathological alterations resembling Alzheimer disease. Acta Pharmacol Sin 2004, 25, (4), 452-7.) Thus, ER beta-selective ligands can affect mood and protect against neurodegenerative diseases. ER beta-selective ligands are also reported to have anti-depressive effects in a mouse model (Walf, A. A.; Rhodes, M. E.; Frye, C. A., Antidepressant effects of ER beta-selective estrogen receptor modulators in the forced swim test. Pharmacol Biochem Behav 2004, 78, (3), 523-9.)

Effects of Estrogens Acting Through ER beta in Lung—Some lung cancers contain ERβ (Stabile, L. P.; Davis, A. L.; Gubish, C. T.; Hopkins, T. M.; Luketich, J. D.; Christie, N.; Finkelstein, S.; Siegfried, J. M., Human non-small cell lung tumors and cells derived from normal lung express both estrogen receptor alpha and beta and show biological responses to estrogen. Cancer Res 2002, 62, (7), 2141-50.) ERβ-selective ligands can have beneficial effects in lung cancer.

ER ligands can be prepared which exhibit fluorescence. Such labeled ER ligands can be employed for imaging, visualization or detection of ER in normal or pathogenic tissue or cells, or tissue or cell extracts. Fluorescent ligands which also exhibit selective interaction with ERs (subtypes or variants) can be employed for the selective imaging, visualization or detection of these ERs in tissues, cells or cell extracts. Fluorescence detection will be selective for ER subtype or for ER variant. Fluorescent ligands of this invention are put in contact with the test tissue, cell or cell extract and treated samples are examined by conventional methods for fluorescence. The selective detection of ER by subtype in breast, ovarian, uterine, cervical and prostate cancers, in pituitary and hypothalamic tumors, in uterine, vascular, and bone pathologies, and in fertility disorders in both males and females can be employed for diagnosis of disorders or in determining the optimal treatment for a given disorder or pathology. ER ligands of this invention can be provided with a label and as such can be employed for imaging or visualization of ER (by subtype, variant and/or tissue or cell distribution) in cultured cells or in tissue samples, e.g., frozen tissue section, and can be employed for assay of tumor cells or tissue or for assay of normal tissue. Fluorescent ER ligands of this invention can be employed in imaging of ER in human and animal cells and tissue, including all mammalian cells and tissue that express ER. Fluorescent ER ligands of this invention which exhibit selective interaction with ERs (e.g., selective binding affinity for different ER subtypes) can be employed for selective imaging of ER subtypes in cells or tissue. Fluorescent ER ligands of this invention can also be used to analyze the cell or tissue distribution of normal ER as well as ER mutants or variants.

The subtype-selective ER ligands of this invention can also be of general use in the investigation of ER and its functions. These ligands can be employed to better understand structure and conformation of ER (both subtypes) and to elucidate how ER subtypes interact with other molecules and to relate structure, conformation and interaction with other molecules to ER function. The subtype-selective ER ligands of this invention are also of general use in drug discovery and development for obtaining additional ER ligands having pharmaceutical use.

This invention is directed to pharmaceutically acceptable compounds, salts, steroisomers and prodrugs of the ER ligands of various structures disclosed herein. Acid addition salts are prepared by contacting compounds having appropriate basic groups therein with an acid whose anion is generally considered suitable for human or animal consumption. Pharmacologically acceptable acid addition salts include, but are not limited to the hydrochloride, hydrobromide, hydroiodide, sulfate, phosphate, acetate, propionate, lactate, maleate, malate, succinate, and tartrate salts. All of these salts can be prepared by conventional means by reacting, for example, the selected acid with the selected basic compound. Base addition salts are analogously prepared by contacting compounds having appropriate acidic groups therein with a base whose cation is generally considered to be suitable for human or animal consumption. Pharmacologically acceptable base addition salts, include but are not limited to ammonium, amine and amide salts.

Pharmaceutically acceptable esters of compounds of this invention are prepared by conventional methods, for example by reaction with selected acids. Pharmaceutically acceptable esters include but are not limited to carboxylic acid esters RCOO-D (where D is a cationic form of a compound of this invention and where R is H, alkyl or aryl groups).

This invention is also directed to prodrugs and derivatives which on being metabolized will result in any of the ER ligands of this invention. For example, alkoxy or acetate groups can be metabolized to hydrogens. Labile substituents may be protected employing conventional and pharmaceutically acceptable protecting groups removable on metabolism. Pharmaceutically active compounds may be derivatized by conventional methods to provide for extended metabolic half-life, to enhance solubility in a given carrier, to provide for or facilitate slow-release or timed-release or enhance or affect other drug delivery properties.

Pharmaceutical compositions according to the present invention comprise one or more ER ligands of this invention in association with a pharmaceutically acceptable carrier or excipient adapted for use in human or veterinary medicine. Such compositions may be prepared for use in conventional manner in admixture with one or more physiologically acceptable carriers or excipients. The compositions may optionally further contain one or more other therapeutic agents which may, if desired, be known ER ligands (agonists, antagonists and/or mixed agonist-antagonist as appropriate). ER ligands are present in these pharmaceutical compositions in an amount or in a combined amount sufficient to elicit a measurable positive effect on a symptom or condition associated with an estrogen-dependent disorder or physiological condition on administration to an individual suffering from the symptom or disorder. Preferred ER ligands of this invention elicit such a measurable positive effect and exhibit selective effect on an ER subtype or variant.

The ER ligands according to the invention may be formulated for oral, buccal, parenteral, topical or rectal administration. In particular, the ER ligands according to the invention may be formulated for injection or for infusion and may be presented in unit dose form in ampules or in multidose containers with an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form for constitution with a suitable vehicle, e.g. sterile, pyrogen-free water, before use. The pharmaceutical compositions according to the invention may also contain other active ingredients, such as antimicrobial agents, or preservatives.

In general, pharmaceutical compositions of this invention can contain from 0.001-99% (by weight) of one or more of the ER ligands disclosed herein. ER ligands may be provided as pure regioisomers or as a mixture of regioisomers; also as pure stereoisomers and a mixture of stereoisomers. Analogously ER ligands may be provided as a mixture of enantiomeric forms or as a purified enantiomer.

The invention further provides a process for preparing a pharmaceutical composition which comprises bringing a ER ligand of the invention into association with a pharmaceutically acceptable excipient or carrier. The carrier or excipient being selected as is known in the art for compatibility with the desired means of administration, for compatibility with the selected ER ligands and to minimize detrimental effects to the patient.

For administration by injection or infusion, the daily dosage as employed for treatment of an adult human of approximately 70 kg body weight will range from 0.2 mg to 10 mg, preferably 0.5 to 5 mg, which can be administered in 1 to 4 doses, for example, depending on the route of administration and the clinical condition of the patient. These formulations also include formulations in dosage units. This means that the formulations are present in the form of a discrete pharmaceutical unit, for example, as tablets, dragees, capsules, caplets, pills, suppositories or ampules. The active compound content of each unit is a fraction or a multiple of an individual dose. The dosage units can contain, for example, 1, 2, 3 or 4 individual doses or ½, ⅓ or ¼ of an individual dose. An individual dose preferably contains the amount of active compound which is given in one administration and which usually corresponds to a whole, one half, one third or one quarter of a daily dose.

The magnitude of a prophylactic or therapeutic dose of a particular compound will, of course, vary with the nature of the severity of the condition to be treated, the particular ER ligand compound and its route of administration. It will also vary according to the age, weight and response of the individual patient.

The compounds of the present invention are preferably formulated prior to administration. The present pharmaceutical formulations are prepared by known procedures using well-known and readily available ingredients. In making the compositions of the present invention, the active ingredient will usually be mixed with a carrier, or diluted by a carrier, or enclosed within a carrier which may be in the form of a capsule, sachet, paper or other container. When the carrier serves as a diluent, it may be a solid, semi-solid or liquid material which acts as a vehicle, excipient or medium for the active ingredient. The compositions can be in the form of tablets, pills, powders, lozenges, sachets, cachets, elixirs, suspensions, emulsions, solutions, syrups, aerosols (as a solid or in a liquid medium), ointments containing for example up to 10% by weight of the active compound, soft and hard gelatin capsules, suppositories, sterile injectable solutions and sterile packaged powders.

Some examples of suitable carriers, excipients, and diluents include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, methyl cellulose, methyl and propylhydroxybenzoates, talc, magnesium stearate and mineral oil. The formulations can additionally include lubricating agents, wetting agents, emulsifying and suspending agents, preserving agents, sweetening agents or flavoring agents. The compositions of the invention may be formulated so as to provide quick, sustained or delayed release of the active ingredient after administration to the patient by employing procedures well known in the art.

The compositions are preferably formulated in a unit dosage form, each dosage containing from about 0.5 to about 150 mg, more usually about 0.1 to about 10 mg, of the active ingredient. The term “unit dosage form” refers to physically discrete units suitable as unitary dosages for human subjects and other mammals, each unit containing a predetermined quantity of active material calculated to produce the desired therapeutic effect, in association with a suitable pharmaceutical carrier.

As a pH adjusting reagent for preparing the pharmaceutical composition, any allowed for preparing medicines can be used, including but not limited to hydrochloric acid-sodium hydroxide, acetic acid-sodium acetate, glycine-sodium chloride-hydrochloric acid, potassium dihydrogenphosphate-disodium hydrogenphosphate, potassium hydrogenphthalate-sodium hydroxide, sodium secondary citrate-hydrochloric acid, sodium dihydrogen-phosphate-disodium hydrogen phosphate, sodium dihydrogenphosphate-dipotassium hydrogen-phosphate, potassium dihydrogenphosphate-dipotassium hydrogenphosphate, tartaric acid-sodium tartrate, lactic acid-sodium lactate, sodium barbital-sodium acetate-hydrochloric acid, succinic acid-boric acid, potassium primary citrate-sodium hydroxide, sodium primary citrate-borax, disodium hydrogenphosphate-citric acid, sodium acetate-hydrochloric acid, glutamic acid-sodium hydroxide, and aspartic acid-sodium hydroxide. Among them, hydrochloric acid-sodium hydroxide, acetic acid-sodium acetate, glycine-sodium chloride-hydrochloric acid, tartaric acid-sodium tartrate, lactic acid-sodium lactate, sodium acetate-hydrochloric acid, glutamic acid-sodium hydroxide, and aspartic acid-sodium hydroxide.

This invention is further directed to therapeutic methods employing the ER ligands of this invention and pharmaceutical compositions containing them in the treatment of estrogen-dependent or estrogen-related disorders or physiological conditions. These methods comprise a step of administering to a patient having the disorder or symptoms thereof a pharmaceutical composition comprising one or a mixture of the ER ligands of this invention where the ER ligand or mixture of ligands is present in the composition at a level or a combined level sufficient to effect a positive biological response. The present invention provides ER ligands that can be used in place of or in combination with currently known pharmaceuticals active in estrogen-dependent or estrogen-related disorders. Certain ER ligands of this invention can exhibit improved properties (enhanced activity and/or decreased undesired side-effects) for treatment of estrogen-dependent and estrogen-responsive disorders.

When a group of substituents is disclosed herein, it is understood that all individual members of those groups and all subgroups, including any isomers and enantiomers of the group members, and classes of compounds that can be formed using the substituents are disclosed separately. When a Markush group or other grouping is used herein, all individual members of the group and all combinations and subcombinations possible of the group are intended to be individually included in the disclosure. When a compound is described herein such that a particular isomer or enantiomer of the compound is not specified, for example, in a formula or in a chemical name, that description is intended to include each isomers and enantiomer of the compound described individual or in any combination. Specific names of compounds are intended to be exemplary, as it is known that one of ordinary skill in the art can name the same compounds differently. Every compound, component, formulation or combination of components described or exemplified herein can be used to practice the invention, unless otherwise stated. Whenever a range is given in the specification, for example, a temperature range, a time range, or a composition range, all intermediate ranges and subranges, as well as all individual values included in the ranges given are intended to be included in the disclosure.

All patents and publications mentioned in the specification are indicative of the levels of skill of those skilled in the art to which the invention pertains. References cited herein are incorporated by reference herein in their entirety to indicate the state of the art as of their filing date and it is intended that this information can be employed herein, if needed, to exclude specific embodiments that are in the prior art. For example, when a compound is claimed, it should be understood that compounds known in the art including the compounds for which an enabling disclosure is provided in the references disclosed herein are not intended to be included in the claim.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms.

The invention illustratively described herein suitably may be practiced in the absence of any element or elements, limitation or limitations which is not specifically disclosed herein.

One of ordinary skill in the art will appreciate that starting materials, reagents, synthetic methods, purification methods, analytical methods, cell expression systems, expression vectors, and biological assays other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

All references cited herein are hereby incorporated by reference in their entirety. Specific definitions provided in this specification take precedence over those in the cited references, although cited references may be employed to indicate the art-known meaning of a term employed herein Some references provided herein are incorporated by reference to provide details concerning sources of starting materials, additional starting materials, additional reagents, additional methods of synthesis, additional methods of analysis and additional uses of the invention.

The following examples are illustrative and not intended to limit the scope of the invention.

### THE EXAMPLES

### Example 1

### General Synthetic Methods

Materials and Methods: All reagents and solvents were obtained from commercial sources. Methylene chloride and tetrahydrofuran were obtained immediately prior to use from a solvent dispensing system (SDS) based on a design developed by Pangborn et al. The reactions were performed under a nitrogen atmosphere unless otherwise noted. Reactions were monitored by thin layer chromatography (TLC), performed on 0.25-mm silica gel plastic plates containing F₂₅₄ indicator. Visualization was obtained using a UV lamp. Column chromatography was performed using Woelm 32-63 micron silica gel packing. Melting points were obtained on a Thomas Hoover capillary melting point apparatus and are uncorrected.

¹H-NMR spectra were obtained on a Varian Unity 400 MHz or 500 MHz spectrometer. ¹³C-NMR were obtained at 100 MHz or 125 MHz, and ¹⁹F-NMR were obtained at 470 MHz. Chemical shifts are reported downfield in parts per million from TMS utilizing the solvent peaks as the reference. Mass spectra were recorded under electron impact (EI) conditions at 70 eV by the Mass Spectrometry Laboratory at the University of Illinois. Elemental analysis of carbon, hydrogen, and nitrogen was performed by the Microanalytical Service Laboratory at the University of Illinois on an Exter Analytical CE440 analyzer.

General Procedure A for the Alkylation of 4-Methoxy-N-(4-methoxy-phenyl)-benzenesulfonamide 7. One equivalent of sulfonamide 7 was dissolved in DMF and 2-3 equivalents of NaH (60% dispersion in mineral oil) was added carefully and the solution allowed to stir for 10 minutes at room temperature. Five equivalents of the desired alkyl halide was added at room temperature and the temperature was increased to 80-100° C., depending upon the boiling point of the alkyl halide. The solution was stirred for 3 h at the desired temperature. 5 mL of a saturated NH₄Cl solution was added carefully, followed by an additional 5 mL of water. The mixture was extracted with EtOAc (3×20 mL) and the organic layers were combined. The EtOAc was washed with a saturated LiCl (3×20 mL) and dried with Na₂SO₄. The EtOAc was removed by rotary evaporation to yield an oil that was purified by flash chromatography 10% acetone/90% methylene chloride.

General Procedure B for the Reduction of Trifluoromethyl amides. One equivalent of the desired amide was dissolved in THF and 5 equivalents of a 1.0 M solution of BH₃.THF was added slowly at room temperature. The reaction was heated to 60° C. and the solution stirred overnight, or until consumption of starting material by TLC. The solution was allowed to cool to room temperature and 5 mL of a saturated NH₄Cl was added carefully, followed by an additional 5 mL of water. The mixture was extracted with EtOAc (3×20 mL) and the organic layers were combined. The EtOAc was washed with a saturated NaCl solution (2×20 mL) and dried with Na₂SO₄. The EtOAc was removed by rotary evaporation. The amine was obtained as an oil and purified by flash chromatography 10% acetone/90% methylene chloride.

General Procedure C for the Coupling of p-Anisidine with 3,3,3-Trifluoropropionoic acid 15. One equivalent of p-anisidine was dissolved in THF. One equivalent of 3,3,3-trifluoropropionic (15) acid and 5 equivalents of pyridine were added to the solution. To this mixture 1.1 equivalents of DCC was added. The reaction was allowed to react for 24 h or until the consumption of starting material. The precipitates were filtered over a fritted filter and the THF was removed by rotary evaporation. The amides were purified by flash chromatography 50% EtOAc/50% hexane.

General Procedure D for the Reaction of Amines with 4-Methoxybezene-sulfonyl chloride 18. 1.1 equivalents of 4-methoxybenzene-sulfonyl chloride (18) was dissolved in THF, 5 equivalents of pyridine was added to this solution and cooled to 0° C. One equivalent of the desired amine dissolved in a minimal amount of CH₂Cl₂ was then added slowly. The reaction mixture was allowed to warm to room temperature and stirred until the consumption of starting material. Water (10 mL) was added to the reaction solution. The mixture was extracted with EtOAc (3×20 mL) and the organic layers combined. The EtOAc was washed with a saturated solution of NaCl and dried with Na₂SO₄. The EtOAc was removed by rotary evaporation and the sulfonamides were purified by flash chromatography 10% acetone/90% methylene chloride.

General Procedure E for the Demethylation of the N-Alkyl-sulfonamides. One equivalent of the N-alkyl-sulfonamide was dissolved in CH₂Cl₂ and cooled to −78° C. in an isopropyl alcohol/dry ice bath. Six equivalents of a 1.0 M solution of BBr₃ in CH₂Cl₂ was added to the solution at −78° C. The reaction mixture was allowed to slowly warm to room temperature and stirred for 24 h or until the starting material was consumed as indicated by TLC. The mixture was cooled to 0° C. and 2 mL of water followed by 2 mL of methanol was slowly added to the mixture. An additional 10 mL of water was added thereafter the mixture was extracted with EtOAc (3×20 mL) and the organic layers combined. The EtOAc was washed with a saturated NaCl (2×20 m) and dried with Na₂SO₄. The EtOAc was removed by rotary evaporation and the demethylated sulfonamides were purified by preparatory TLC 10% acetone/90% methylene chloride (at least 2 developments).

### Exemplary Syntheses

### 4-Methoxy-N-(4-methoxy-phenyl)-N-methyl-benzenesulfonamide (9a)

Following general procedure A, methyl iodide (8a) was reacted with sulfonamide 7 to yield 9a as a colorless oil. Isolated yield 75%. ¹H-NMR ((CD₃)₂CO 500 MHz) δ 3.10 (s, 3H, Ar—NCH₃), 3.77 (s, 3H, Ar—OCH₃), 3.88 (s, 3H, Ar—OCH₃), 6.84 (AA′XX′, 2H, Ar—H), 7.00 (AA′XX′, 2H, Ar—H), 7.05 (AA′XX′, 2H, Ar—H), 7.46 (AA′XX′, 2H, Ar—H); ¹³C-NMR ((CD₃)₂CO 125 MHz) δ 38.69 (1C, Ar—NCH₃), 55.71 (1C, Ar—OCH₃), 56.07 (1C, Ar—OCH₃), 114.67 (2C, Ar—C), 114.80 (2C, Ar—C), 128.79 (2C, Ar—C), 129.40 (1C, Ar—CSO₂), 130.79 (2C, Ar—C), 135.47 (1C, Ar—CNCH₃), 159.59 (1C, Ar—COCH₃), 164.01 (1C, Ar—COCH₃); LRMS m/z 307.1 (M⁺); HRMS (C₁₅H₁₇NO₄S) calcd 307.0878 found 307.0881.

### N-Ethyl-4-methoxy-N-(4-methoxy-phenyl)-benzenesulfonamide (9b)

Following general procedure A, ethyl iodide (8b) was reacted with sulfonamide 7 to yield 9b as a colorless oil. Isolated yield 75%. ¹H-NMR (CDCl₃ 500 MHz) δ 1.04 (t, J=7.07 Hz, 3H, NCH₂CH₃), 3.53 (q, J=7.07 Hz, 2H, NCH₂CH₃), 3.78 (s, 3H, Ar—OCH₃), 3.84 (s, 3H, Ar—OCH₃), 6.80 (AA′XX′, 2H, Ar—H), 6.92 (m, 2H, Ar—H), 7.52 (AA′XX′, 2H, Ar—H); ¹³C-NMR (CDCl₃ 125 MHz) δ 14.06 (1C, NCH₂CH₃), 45.68 (1C, NCH₂CH₃), 55.49 (1C, Ar—OCH₃), 55.67 (1C, Ar—OCH₃), 113.97 (2C, Ar—C), 114.23 (2C, Ar—C), 129.86 (2C, Ar—C), 130.20 (1C, Ar—CSO₂), 130.27 (2C, Ar—C), 131.48 (2C, Ar—C), 159.10 (1C, Ar—COCH₃), 162.86 (1C, Ar—COCH₃); LRMS m/z 321.1 (M⁺); HRMS (C₁₆H₁₉NO₄S) calcd 321.1035 found 321.1033.

### 4-Methoxy-N-(4-methoxy-phenyl)-N-propyl-benzenesulfonamide (9c)

Following general procedure A, 1-brompropane (8c) was reacted with sulfonamide 7 to yield 9c as a colorless oil. Isolated yield 92%. ¹H-NMR (CDCl₃ 500 MHz) δ 0.89 (t, J=7.4 Hz, 3H, NCH₂CH₂CH₃), 1.41 (sext, J=7.29 Hz, 2H, NCH₂CH₂CH₃), 3.44 (t, J=7.07 Hz, 2H, NCH₂CH₂CH₃), 6.81 (AA′XX′, 2H, Ar—H), 6.91 (AA′XX′, 2H, Ar—H), 6.94 (AA′XX′, 2H, Ar—H), 7.52 (AA′XX′, 2H, Ar—H); ¹³C-NMR (CDCl₃ 125 MHz) δ 11.15 (1C, NCH₂CH₂CH₃), 21.68 (1C, NCH₂CH₂CH₃), 52.58 (1C, NCH₂CH₂CH₃), 55.58 (1C, Ar—OCH₃), 55.72 (1C, Ar—OCH₃), 114.03 (2C, Ar—C), 114.32 (1C, Ar—C), 129.97 (1C, Ar—C), 130.26 (1C, Ar—C), 130.56 (1C, Ar—CSO₂), 132.04 (1C, Ar—CN—Pr), 159.17 (1C, Ar—COCH₃), 162.95 (1C, Ar—COCH₃); LRMS m/z 335.1 (M⁺); HRMS (C₁₇H₂₁NO₄S) calcd 335.1191 found 335.1198.

### N-Butyl-4-methoxy-N-(4-methoxy-phenyl)-benzenesulfonamide (9d)

Following general procedure A, 1-bromobutane (8d) was reacted with sulfonamide 7 to yield 9d as a colorless oil. Isolated yield 97%. ¹H-NMR (CDCl₃ 500 MHz) δ 0.84 (t, J=7.07 Hz, 3H, NCH₂CH₂CH₂CH₃), 1.34 (m, 4H, NCH₂CH₂CH₂CH₃), 3.46 (t, J=6.86 Hz, 2H, NCH₂CH₂CH₂CH₃), 3.78 (s, 3H, Ar—OCH₃), 3.85 (s, 3H, Ar—OCH₃), 6.80 (AA′XX′, 2H, Ar—H), 6.90 (AA′XX′, 2H, Ar—H), 6.93 (AA′XX′, 2H, Ar—H), 7.51 (AA′XX′, 2H, Ar—H); ¹³C-NMR (CDCl₃ 125 MHz) δ 13.72 (1C, NCH₂CH₂CH₂CH₃), 19.72 (1C, NCH₂CH₂CH₂CH₃), 30.38 (1C, NCH₂CH₂CH₂CH₃), 50.48 (1C, NCH₂CH₂CH₂CH₃), 55.51 (1C, Ar—OCH₃), 55.68 (1C, Ar—OCH₃), 113.99 (2C, Ar—C), 114.26 (2C, Ar—C), 129.93 (1C, Ar—C), 130.18 (2C, Ar—C), 130.39 (1C, Ar—CSO₂), 131.97 (1C, Ar—CN-Bu), 159.11 (1C, Ar—COCH₃), 162.91 (1C, Ar—COCH₃); LRMS m/z 349.2 (M⁺); HRMS (C₁₈H₂₃NO₄S) calcd 349.1341 found 349.1343.

### N-Isobutyl-4-methoxy-N-(4-methoxy-phenyl)-benzenesulfonamide (9e)

Following general procedure A, isobutyl-bromide (8e) was reacted with sulfonamide 7 to yield 9e as a colorless oil. Isolated yield 98%. ¹H-NMR (CDCl₃ 500 MHz) δ 0.89 (d, J=6.65 Hz, 6H, NCH₂CH(CH₃)₂), 1.54 (non., J=6.86 Hz, 1H, NCH₂CH(CH₃)₂), 3.24 (d, J=7.29 Hz, NCH₂CH(CH₃)₂), 3.78 (s, 3H, Ar—OCH₃), 3.84 (s, 3H, Ar—OCH₃), 6.79 (AA′XX′, 2H, Ar—H), 6.90 (AA′XX′, 2H, Ar—H), 6.94 (AA′XX′, 2H, Ar—H), 7.49 (AA′XX′, 2H, Ar—H); ¹³C-NMR (CDCl₃ 125 MHz) δ 19.99 (2C, NCH₂CH(CH₃)₂), 26.79 (1C, NCH₂CH(CH₃)₂), 55.49 (1C, Ar—OCH₃), 55.67 (1C, Ar—OCH₃), 58.11 (1C, NCH₂CH(CH₃)₂), 113.94 (2C, Ar—C), 114.20 (2C, Ar—C), 129.89 (2C, Ar—C), 129.94 (2C, Ar—C), 130.07 (1C, ArCSO₂), 132.15 (1C, Ar—CN-i-Bu), 158.97 (1C, Ar—COCH₃) 162.85 (1C, Ar—COCH₃); LRMS m/z 349.2 (M⁺); HRMS (C₁₈H₂₃NO₄S) calcd 349.1341 found 349.1344.

### 4-Methoxy-N-(4-methoxy-phenyl)-N-pentyl-benzenesulfonamide (9f)

Following general procedure A, 1-bromopentane (8f) was reacted with sulfonamide 7 to yield 9f as a colorless oil. Isolated yield 60%. ¹H-NMR ((CD₃)₂CO 500 MHz) δ 0.83 (t, J=7.07 Hz, 3H, NCH₂CH₂CH₂CH₂CH₃), 1.28 (m, 6H, NCH₂CH₂CH₂CH₂CH₃), 3.51 (t, J=6.86 Hz, 2H, NCH₂CH₂CH₂CH₂CH₃), 3.78 (s, 3H, Ar—OCH₃), 3.88 (s, 3H, Ar—OCH₃), 6.86 (AA′XX′, 2H, Ar—H), 6.96 (AA′XX′, 2H, Ar—H), 7.05 (AA′XX′, 2H, Ar—H), 7.51 (AA′XX′, 2H, Ar—H), ¹³C-NMR (CDCl₃ 125 MHz) δ 14.18 (1, NCH₂CH₂CH₂CH₂CH₃), 22.78 (1C, NCH₂CH₂CH₂CH₂CH₃), 28.55 (1C, NCH₂CH₂CH₂CH₂CH₃), 51.13 (1C, NCH₂CH₂CH₂CH₂CH₃), 53.25 (1C, NCH₂CH₂CH₂CH₂CH₃), 55.73 (1C, Ar—OCH₃), 56.09 (1C, Ar—OCH₃), 114.82 (2C, Ar—C), 114.84 (2C, Ar—C), 130.59 (2C, Ar—C), 130.89 (2C, Ar—C), 131.41 (1C, Ar—CSO₂), 132.91 (1C, Ar—CN-Pent), 159.99 (1C, Ar—COCH₃), 163.89 (1C, Ar—COCH₃); LRMS m/z 363.2 (M⁺); HRMS (C₁₉H₂₅NO₄S) calcd 363.1504 found 363.1503.

### N-Isopropyl-4-methoxy-N-(4-methoxy-phenyl)-benzenesulfonamide (9g)

Following general procedure A, 2-iodopropane (8g) was reacted with sulfonamide 7 to yield 9g as a colorless oil. Isolated yield 71%. ¹H-NMR (CDCl₃ 500 MHz) δ 1.02 (d, J=6.86 Hz, 6H, NCH(CH₃)₂), 3.79 (s, 3H, Ar—OCH₃), 3.85 (s, 3H, Ar—OCH₃), 4.57 (sept, J=6.65 Hz, 1H, NCH(CH₃)₂), 6.82 (AA′XX′, 2H, Ar—H), 6.91 (AA′XX′, 2H, Ar—H), 6.94 (AA′XX′, 2H, Ar—H), 7.65 (AA′XX′, 2H, Ar—H); ¹³C-NMR (CDCl₃ 125 MHz) δ 22.16 (2C, NCH(CH₃)₂), 50.99 (1C, NCH(CH₃)₂), 55.51 (1C, Ar—OCH₃), 55.68 (1C, Ar—OCH₃), 114.04 (2C, Ar—C), 114.10 (2C, Ar—C), 127.61 (1C, Ar—CSO₂), 129.57 (2C, Ar—C), 133.49 (1C, Ar—CN-i-Pr), 133.73 (2C, Ar—C), 159.69 (1C, Ar—COCH₃), 162.70 (1C, Ar—COCH₃); LRMS m/z 335.2 (M⁺); HRMS (C₁₇H₂₁NO₄S) calcd 335.1191 found 335.1193.

### N-sec-Butyl-4-methoxy-N-(4-methoxy-phenyl)-benzenesulfonamide (9h)

Following general procedure A, 2-bromobutane (8h) was reacted with sulfonamide 7 to yield 9h as a colorless oil. Isolated yield 72%. ¹H-NMR (CDCl₃ 500 MHz) δ 0.94 (t, J=7.40 Hz, 3H, NCHCH₃(CH₂CH₃)), 1.00 (d, J=6.86 Hz, 3H, NCHCH₃(CH₂CH₃)), 1.19 (dpent, J=13.72, 7.07 Hz, 1H, NCHCH₃(CH₂CH₃)), 1.37 (dpent, J=13.94, 7.50 Hz, 1H, NCHCH₃(CH₂CH₃)), 3.79 (s, 3H, Ar—OCH₃), 3.85 (s, 3H, Ar—OCH₃), 4.27 (sextet, J=6.65 Hz, 1H, NCHCH₃(CH₂CH₃)), 6.81 (AA′XX′, 2H, Ar—H), 6.92 (m, 4H, Ar—H), 7.63 (AA′XX′, 2H, Ar—H); ¹³C-NMR (CDCl₃ 125 MHz) δ 11.60 (1C, NCHCH₃(CH₂CH₃)), 19.96 (1C, NCHCH₃(CH₂CH₃)), 28.71 (1C, NCHCH₃(CH₂CH₃)), 55.51 (1C, Ar—OCH₃), 55.68 (1C, Ar—OCH₃), 57.21 (1C, NCHCH₃(CH₂CH₃)), 113.96 (2C, Ar—C), 114.06 (2C, Ar—C), 127.71 (1C, Ar—CSO₂), 129.59 (2C, Ar—C), 133.47 (1C, Ar—CN-s-Butyl), 133.57 (2C, Ar—C), 159.60 (1C, Ar—COCH₃), 162.63 (Ar—COCH₃); LRMS m/z 349.1 (M⁺); HRMS (C₁₈H₂₃NO₄S) calcd 349.1348 found 349.1354.

### 4-Methoxy-N-(4-methoxy-phenyl)-N-(1-methyl-butyl)-benzenesulfonamide (9i)

Following general procedure A, 2-bromopentane (8i) was reacted with sulfonamide 7 to yield 9i as a colorless oil. Isolated yield 77%. ¹H-NMR (CDCl₃ 500 MHz) δ 0.88 (t, J=7.29 Hz, 3H, NCHCH₃(CH₂CH₂CH₃)), 1.00 (d, J=6.86 Hz, 3H, NCHCH(CH₂CH₂CH₃)), 1.37 (m, 4H, NCHCH₃(CH₂CH₂CH₃)), 3.79 (s, 3H, Ar—OCH₃), 3.85 (s, 3H, Ar—OCH₃), 4.38 (m, 1H, NCHCH₃(CH₂CH₂CH₃), 6.82 (AA′XX′, 2H, Ar—H), 6.92 (m, 4H, Ar—H), 7.63 (AA′XX′, 2H, Ar—H); ¹³C-NMR (CDCl₃ 125 MHz) δ 14.02 (1C, NCHCH₃(CH₂CH₂CH₃)), 19.98 (1C, NCHCH₃(CH₂CH₂CH₃)), 29.84 (1C, NCHCH₃(CH₂CH₂CH₃)), 37.88 (1C, NCHCH₃(CH₂CH₂CH₃)), 55.20 (1C, Ar—OCH₃), 55.45 (1C, NCHCH₃(CH₂CH₂CH₃), 55.68 (1C, Ar—OCH₃), 113.95 (2C, Ar—C), 114.06 (2C, Ar—C), 129.59 (1C, Ar—CSO₂), 129.67 (2C, Ar—C), 133.46 (1C, Ar—CN(2-Pentyl)), 133.60 (2C, Ar—C), 159.60 (1C, Ar—COCH₃), 162.63 (1C, Ar—COCH₃); LRMS m/z 363.1 (M⁺); HRMS (C₁₉H₂₅NO₄S) calcd 363.1504 found 363.1502.

### N-(1-Ethyl-propyl)-4-methoxy-N-(4-methoxy-phenyl)-benzenesulfonamide (9j)

Following general procedure A, 3-bromopentane (8j) was reacted with sulfonamide 7 to yield 9j as a colorless oil. Isolated yield 78%. ¹H-NMR (CDCl₃ 500 MHz) δ 0.95 (t, J=7.40 Hz, 6H, NCH(CH₂CH₃)₂), 1.24 (m, 2H, NCH(CH₂CH₃)₂), 1.33 (m, 2H, NCH(CH₂CH₃)₂), 3.79 (s, 3H, Ar—OCH₃), 3.84 (s, 3H, Ar—OCH₃), 4.02 (m, 1H, NCH(CH₂CH₃)₂), 6.80 (AA′XX′, 2H, Ar—H), 6.88 (AA′XX′, 2H, Ar—H), 6.94 (AA′XX′, 2H, Ar—H), 7.58 (AA′XX′, 2H, Ar—H); ¹³C-NMR (CDCCl₃ 125 MHz) δ 11.87 (2C, NCH(CH₂CH₃)₂), 26.45 (2C, NCH(CH₂CH₃)₂), 55.49 (1C, Ar—OCH₃), 55.65 (1C, Ar—OCH₃), 64.16 (1C, NCH(CH₂CH₃)₂), 113.80 (2C, Ar—C), 114.05 (2C, Ar—C), 127.76 (1C, Ar—CSO₂), 129.65 (2C, Ar—C), 133.41 (1C, Ar—CN(3-Pentyl)), 133.47 (2C, Ar—C), 159.54 (1C, Ar—COCH₃), 162.56 (1C, Ar—COCH₃); LRMS m/z 363.2 (M⁺); HRMS (C₁₉H₂₅NO₄S) calcd 363.1504 found 363.1507.

### 2,2,2-Trifluoro-N-(4-methoxy-phenyl)-acetamide (13)

One equivalent of p-anisidine 11 was dissolved in CH₂Cl₂ and cooled to 0° C. Trifluoroacetic anhydride 12 (4 equivalents) and 5 equivalents of pyridine were added and the mixture was allowed to warm to room temperature. The solution was stirred for 3 hours, at which time 10 mL of H₂O was added to the reaction. The mixture was extracted with EtOAc (3×25 mL) and the organic layers combined. The EtOAc was washed with a solution of saturated NaCl (2×25 mL) and dried with Na₂SO₄. The EtOAc was removed by rotary evaporation and the product was carried on to the next step without further purification. ¹H-NMR ((CD₃)₂CO 500 MHz) δ 3.79 (s, 3H, Ar—OCH₃), 6.95 (AA′XX′, 2H, Ar—H), 7.62 (AA′XX′, 2H, Ar—H), 10.11 (bs, 1H, NHC═O); ¹³C-NMR ((CD₃)₂CO 125 MHz) δ 55.70 (1C, Ar—OCH₃), 114.92 (2C, Ar—C), 123.23 (1C, Ar—CNHC═O), 123.32 (2C, Ar—C), 129.13 (q, J=280.79 Hz, 1C, NC═OCF₃), 158.43 (1C, Ar—COCH₃); ¹⁹F-NMR (CDCl₃ 470 MHz) δ −76.55 (3F, NC═OCF₃); LRMS m/z 219.0 (M⁺); HRMS (C₉H₈F₃NO₂) calcd 219.0507 found 219.0508.

### (4-Methoxy-phenyl)-(2,2,2-trifluoro-ethyl)-amine (14)

Following general procedure B, Compound 13 was reduced with borane to yield 14 as a colorless oil. Isolated yield 89%, from Compound 11. ¹H-NMR (CDCl₃ 500 MHz) δ 3.72 (q, J=6.86 Hz, 2H, NHCH₂CF₃), 3.73 (bs, 1H, NHCH₂CF₃), 3.78 (s, 3H, Ar—OCH₃), 6.68 (AA′XX′, 2H, Ar—H), 6.84 (AA′XX′, 2H, Ar—H); ¹³C-NMR (CDCl₃ 125 MHz) δ 46.76 (q, J=32.22 Hz, 1C, NHCH₂CF₃), 55.72 (1C, Ar—OCH₃), 114.75 (2C, Ar—C), 115.01 (2C, Ar—C), 125.42 (q, J=279.87 Hz, 1C, NHCH₂CF₃), 140.53 (1C, Ar—CNHCH₂CF₃), 153.22 (1C, Ar—COCH₃); LRMS m/z 205.1 (M⁺); HRMS (C₉H₈F₃NO) calcd 205.0714 found 205.0710.

### 3,3,3-Trifluoro-N-(4-methoxy-phenyl)-propionamide (16)

Following general procedure C, 11 was coupled with 3,3,3-trifluoropropionic acid 15 to yield 16 as a white solid. Isolated yield 71%. ¹H-NMR ((CD₃)₂CO 500 MHz) δ 3.40 (q, J=10.86 Hz, 2H, NC═OCH₂CF₃), 3.76 (s, 3H, Ar—OCH₃), 6.88 (AA′XX′, 2H, Ar—H), 7.53 (AA′XX′, 2H, Ar—H), 9.35 (bs, 1H, NHC═O); ¹³C-NMR ((CD₃)₂CO 125 MHz) δ 41.78 (q, J=28.54 Hz, 1C, NC═OCH₂CF₃), 55.58 (1C, Ar—OCH₃), 114.69 (2C, Ar—C), 121.91 (2C, Ar—C), 125.73 (q, J=276.19 Hz, 1C, NC═OCH₂CF₃), 132.57 (1C, Ar—CNHC═O), 157.25 (1C, Ar—COCH₃), 161.60 (q, J=2.76 Hz, 1C, NC═OCH₂CF₃); ¹⁹F-NMR ((CD₃)₂CO 470 MHz) δ −63.96 (3F, NC═OCH₂CF₃) LRMS m/z 233.1 (M⁺); HRMS (C₁₀H₁₀F₃NO₂) calcd 233.0664 found 233.0668.

### (4-Methoxy-phenyl)-(3,3,3-trifluoro-propyl)-amine (17)

Following general procedure B, 16 was reduced with borane to yield 17 as a colorless oil. ¹H-NMR (CDCl₃ 500 MHz) δ 2.40 (qt, J=10.93, 7.07 Hz, 2H, NHCH₂CH₂CF₃), 3.41 (t, J=6.97 Hz, 2H, NHCH₂CH₂CF₃), 3.53 (bs, 1H, Ar—NHCH₂CH₂CF₃), 3.77 (s, 3H, Ar—OCH₃), 6.61 (AA′XX′, 2H, Ar—H), 6.84 (AA′XX′, 2H, Ar—H); ¹³C-NMR (CDCl₃ 125 MHz) δ 33.52 (q, J=27.62 Hz, 1C, NCH₂CH₂CF₃), 38.14 (q, J=3.68 Hz, 1C, NCH₂CH₂CF₃), 55.80 (1C, Ar—OCH₃), 114.55 (2C, Ar—C), 115.13 (2C, Ar—C), 126.76 (q, J=277.11 Hz, 1C, NHCH₂CH₂CF₃), 141.23 (1C, Ar—CNHCH₂CH₂CF₃), 152.75 (1C, Ar—COCH₃); ¹⁹F-NMR (CDCl₃ 470 MHz) δ −65.36 (3F, NCH₂CH₂CF₃); LRMS m/z 219.1 (M⁺); HRMS (C₁₀H₁₂F₃NO) calcd 219.0871 found 219.0871.

### 4-Methoxy-N-(4-methoxy-phenyl)-N-(2,2,2-trifluoro-ethyl)benzenesulfonamide (19a)

Following general procedure D, 14 was reacted with 18 to yield 19a as a colorless oil. Isolated yield 77%. ¹H-NMR ((CD₃)₂CO 500 MHz) δ 3.78 (s, 3H, Ar—OCH₃), 3.88 (s, 3H, Ar—OCH₃), 4.41 (q, J=8.65 Hz, 2H, NCH₂CF₃), 6.86 (AA′XX′, 2H, Ar—H), 7.04 (m, 4H, Ar—H), 7.57 (AA′XX′, 2H, Ar—H); ¹³C-NMR ((CD₃)₂CO 125 MHz) δ 55.76 (1C, Ar—OCH₃), 56.15 (1C, Ar—OCH₃), 52.79 (q, J=34.06 Hz, 1C, NCH₂CF₃), 115.07 (4C, Ar—C), 125.35 (q, J=278.95 Hz, 1C, NCH₂CF₃), 130.81 (2C, Ar—C), 130.85 (1C, Ar—CSO₂), 131.30 (2C, Ar—C), 132.90 (1C, Ar—CNCH₂CF₃), 160.50 (1C, Ar—COCH₃), 164.40 (1C, Ar—COCH₃); ¹⁹F-NMR ((CD₃)₂CO 470 MHz) δ −71.69 (3F, NCH₂CF₃); LRMS m/z 375.1 (M⁺); HRMS (C₁₆H₁₆F₃NO₄S) calcd 375.0752 found 375.0752.

### 4-Methoxy-N-(4-methoxy-phenyl)-N-(3,3,3-trifluoro-propyl)benzenesulfonamide (19b)

Following general procedure D, 17 was reacted with 18 to yield 19b as a colorless oil. Isolated yield 92%. ¹H-NMR (CDCl₃ 500 MHz) δ 2.33 (m, 2H, NCH₂CH₂CF₃), 3.71 (m, 2H, NCH₂CH₂CH₃), 3.78 (s, 3H, Ar—OCH₃), 3.84 (s, 3H, Ar—OCH₃), 6.82 (AA′XX′, 2H, Ar—H), 6.92 (m, 4H, Ar—H), 7.50 (AA′XX′, 2H, Ar—H); ¹³C-NMR (CDCl₃ 125 MHz) δ 33.54 (q, J=28.54 Hz, 1C, NCH₂CH₂CF₃), 44.58 (q, J=3.68 Hz, 1C, NCH₂CH₂CF₃), 55.51 (1C, Ar—OCH₃), 55.69 (1C, Ar—OCH₃), 114.18 (2C, Ar—C), 114.56 (2C, Ar—C), 129.22 (1C, Ar—CSO₂), 129.94 (2C, Ar—C), 130.06 (2C, Ar—C), 131.18 (1C, Ar—CNCH₂CH₂CF₃), 159.48 (1C, Ar—COCH₃), 163.24 (1C, Ar—COCH₃); ¹⁹F-NMR (CDCl₃ 470 MHz) δ −65.67 (3F, NCH₂CH₂CF₃); LRMS m/z 389.1 (M⁺); HRMS (C₁₇H₁₈F₃NO₄S) calcd 389.0909 found 389.0914.

### 4-Hydroxy-N-(4-hydroxy-phenyl)-N-methyl-benzenesulfonamide (20a)

Following general procedure E, 9a was demethylated to yield 20a as a white solid. Isolated yield 52%. ¹H-NMR ((CD₃)₂CO 500 MHz) δ 3.07 (s, 3H, Ar—NCH₃), 6.74 (AA′XX′, 2H, Ar—H), 6.89 (AA′XX′, 2H, Ar—H), 6.94 (AA′XX′, 2H, Ar—H), 7.38 (AA′XX′, 2H, Ar—H); ¹³C-NMR ((CD₃)₂CO 125 MHz) δ 61.13 (1C, Ar—NCH₃), 116.01 (2C, Ar—C), 116.10 (2C, Ar—C), 128.90 (2C, Ar—C), 130.91 (1C, Ar—CSO₂), 131.00 (2C, Ar—C), 134.58 (1C, Ar—CNCH₃), 157.31 (1C, Ar—COH), 162.14 (1C, Ar—COH); LRMS m/z 279.2 (M⁺); HRMS (C₁₃H₁₃NO₄S) calcd 279.0565 found 279.0569; Anal (C₁₃H₁₃NO₄S.0.1H₂O) C, H, N calcd 55.54% C, 4.73% H, 4.98% N found 55.18% C, 4.79% H, 4.88% N.

### N-Ethyl-4-hydroxy-N-(4-hydroxy-phenyl)-benzenesulfonamide (20b)

Following general procedure E, 9b was demethylated to yield 20b as an off-white solid. Isolated yield 52%. ¹H-NMR ((CD₃)₂CO 500 MHz) δ 0.99 (t, J=7.18 Hz, 3H, NCH₂CH₃), 3.53 (q, J=7.07 Hz, 2H, NCH₂CH₃), 6.76 (AA′XX′, 2H, Ar—H), 6.85 (AA′XX′, 2H, Ar—H), 6.94 (AA′XX′, 2H, Ar—H), 7.44 (AA′XX′, 2H, Ar—H); ¹³C-NMR ((CD₃)₂CO 125 MHz) δ 14.32 (1C, NCH₂CH₃); 46.15 (1C, NCH₂CH₃); 116.17 (2C, Ar—C), 116.22 (2C, Ar—C), 130.47 (1C, Ar—CSO₂), 130.79 (2C, Ar—C), 131.10 (2C, Ar—C), 131.70 (1C, Ar—CNCH₂CH₃), 157.79 (1C, Ar—COH), 162.02 (1C, Ar—COH); LRMS m/z 293.1 (M⁺); HRMS (C₁₄H₁₅NO₄S) calcd 293.0722 found 293.0730; Anal (C₁₄H₁₅NO₄S.0.6H₂O) C, H, N calcd 55.29% C, 5.37% H, 4.61% N found 54.98% C, 4.98% H, 4.52% N.

### 4-Hydroxy-N-(4-hydroxy-phenyl)-N-propyl-benzenesulfonamide (20c)

Following general procedure E, 9c was demethylated to yield 20c as a white solid. Isolated yield 52%. ¹H-NMR ((CD₃)₂CO 500 MHz) δ 0.86 (t, J=7.29 Hz, 3H, NCH₂CH₂CH₃), 1.36 (sextet, J=7.07 Hz, 2H, NCH₂CH₂CH₃), 3.45 (t, J=6.97 Hz, 2H, NCH₂CH₂CH₃), 6.76 (AA′XX′, 2H, Ar—H), 6.86 (AA′XX′, 2H, Ar—H), 6.94 (AA′XX′, 2H, Ar—H), 7.42 (AA′XX′, 2H, Ar—H), 8.84 (bs, 2H, Ar—OH); ¹³C-NMR ((CD₃)₂CO 125 MHz) δ 11.22 (1C, NCH₂CH₂CH₃), 22.03 (1C, NCH₂CH₂CH₃), 52.79 (1C, NCH₂CH₂CH₃), 116.10 (1C, Ar—C), 116.12 (1C, Ar—C), 130.20 (1C, Ar—CSO₂), 130.76 (2C, Ar—C), 130.91 (1C, Ar—C), 131.83 (1C, Ar—CN(n-Pr)), 157.57 (1C, Ar—COH), 161.93 (1C, Ar—COH); LRMS m/z 307.2 (M⁺); HRMS (C₁₅H₁₇NO₄S) calcd 307.0878 found 307.0886; Anal (C₁₅H₁₇NO₄S.0.1H₂O) C, H, N calcd 58.27% C, 5.61% H, 4.53% N found 58.00% C, 5.71% H, 4.60% N.

### N-Butyl-4-hydroxy-N-(4-hydroxy-phenyl)-benzenesulfonamide (20d)

Following general procedure E, 9d was demethylated to yield 20d as a white solid. Isolated yield 60%. ¹H-NMR ((CD₃)₂CO 500 MHz) δ 0.83 (t, J=7.18 Hz, 3H, NCH₂CH₂CH₂CH₃), 1.33 (m, 4H, NCH₂CH₂CH₂CH₃), 3.49 (t, J=6.54 Hz, 2H, NCH₂CH₂CH₂CH₃), 6.76 (AA′XX′, 2H, Ar—H), 6.86 (AA′XX′, 2H, Ar—H), 6.94 (AA′XX′, 2H, Ar—H), 7.43 (AA′XX′, 2H, Ar—H), 8.56 (bs, 1H, Ar—OH), 9.27 (bs, 1H, Ar—OH); ¹³C-NMR ((CD₃)₂CO 125 MHz) δ 13.79 (1C, NCH₂CH₂CH₂CH₃), 20.09 (1C, NCH₂CH₂CH₂CH30.85 (1C, NCH₂CH₂CH₂CH₃), 50.64 (1C, NCH₂CH₂CH₂CH₃), 116.13 (4C, Ar—C), 130.14 (1C, Ar—CSO₂), 130.76 (2C, Ar—C), 130.88 (2C, Ar—C), 131.82 (1C, Ar—CN(n-Bu)), 157.67 (1C, Ar—COH), 161.92 (1C, Ar—COH); LRMS m/z 321.0 (M⁺); HRMS (C₁₆H₁₉NO₄S) calcd 321.1035 found 321.1037; Anal (C₁₆H₁₉NO₄S.0.2H₂O) C, H, N calcd 59.13% C, 6.02% H, 4.31% N found 58.88% C, 5.89% H, 4.24% N.

### N-Isobutyl-4-hydroxy-N-(4-hydroxy-phenyl)-benzenesulfonamide (20e)

Following general procedure E, 9e was demethylated to yield 20e as a white solid. Isolated yield 53%. ¹H-NMR ((CD₃)₂CO 500 MHz) δ 0.87 (d, J=6.65 Hz, 6H, NCH₂CH(CH₃)₂), 1.51 (non., J=6.86 Hz, 1H, NCH₂CH(CH₃)₂), 3.27 (d, J=7.29 Hz, 2H, NCH₂CH(CH₃)₂), 6.76 (AA′XX′, 2H, Ar—H), 6.87 (AA′XX′, 2H, Ar—H), 6.93 (AA′XX′, 2H, Ar—H), 7.41 (AA′XX′, 2H, Ar—H), 8.57 (bs, 1H, Ar—OH), 9.33 (bs, 1H, Ar—OH); ¹³C-NMR ((CD₃)₂CO 125 MHz) δ 20.09 (2C, NCH₂CH(CH₃)₂), 27.40 (1C, NCH₂CH(CH₃)₂), 58.53 (1C, NCH₂CH(CH₃)₂), 116.09 (2C, Ar—C), 116.12 (2C, Ar—C), 130.09 (1C, Ar—CSO₂), 130.72 (2C, Ar—C), 130.75 (2C, Ar—C), 132.16 (1C, Ar—CN(i-Bu), 157.63 (1C, Ar—COH), 161.93 (1C, Ar—COH); LRMS m/z 321.0 (M⁺); HRMS (C₁₆H₁₉NO₄S) calcd 321.1035 found 321.1040; Anal (C₁₆H₁₉NO₄S.0.3H₂O) C, H, N calcd 58.81% C, 6.05% H, 4.29% N found 58.58% C, 5.82% H, 4.10% N.

### 4-Hydroxy-N-(4-hydroxy-phenyl)-N-pentyl-benzenesulfonamide (20f)

Following general procedure E, 9f was demethylated to yield 20f as a white solid. Isolated yield 60%. ¹H-NMR ((CD₃)₂CO 500 MHz) δ 0.82 (t, J=7.07 Hz, 3H, N(CH₂)₄CH₃), 1.30 (m, 6H, NCH₂(CH₂)₃CH₃), 3.48 (t, J=6.75 Hz, 2H, NCH₂(CH₂)₃CH₃), 6.76 (AA′XX′, 2H, Ar—H), 6.86 (AA′XX′, 2H, Ar—H), 6.94 (AA′XX′, 2H, Ar—H), 7.43 (AA′XX′, 2H, Ar—H), 8.56 (bs, 1H, Ar—OH), 9.31 (bs, 1H, Ar—OH); ¹³C-NMR ((CD₃)₂CO 125 MHz) δ 14.19 (1C, N(CH₂)₄CH₃), 22.75 (1C, N(CH₂)₃CH₂CH₃), 28.42 (1C, N(CH₂)₂CH₂CH₂CH₃), 29.16 (1C, NCH₂CH₂(CH₂)₂CH₃), 50.94 (1C, NCH₂(CH₂)₃CH₃), 116.10 (2C, Ar—C), 116.13 (2C, Ar—C), 130.15 (1C, Ar—CSO₂), 130.75 (2C, Ar—C), 130.89 (2C, Ar—C), 131.81 (1C, Ar—CN(n-Pent)), 157.68 (1C, Ar—COH), 161.92 (1C, Ar—COH); LRMS m/z 335.1 (M⁺); HRMS (C₁₇H₂₁NO₄S) calcd 335.1191 found 335.1190; Anal (C₁₇H₂₁NO₄S.0.4H₂O) C, H, N calcd 59.59% C, 6.41% H, 4.09% N found 59.24% C, 6.21% H, 3.99% N.

### N-Isopropyl-4-hydroxy-N-(4-hydroxy-phenyl)-benzenesulfonamide (20g)

Following general procedure E, 9g was demethylated to yield 20g as a white solid, mp=158-159° C. Isolated yield 50%. ¹H-NMR ((CD₃)₂CO 500 MHz) δ 0.99 (d, J=6.86 Hz, 6H, NCH(CH₃)₂), 4.50 (sept. J=6.86 Hz, 1H, NCH(CH₃)₂), 6.79 (AA′XX′, 2H, Ar—H), 6.87 (AA′XX′, 2H, Ar—H), 6.95 (AA′XX′, 2H, Ar—H), 7.59 (AA′XX′, 2H, Ar—H), 8.59 (bs, 1H, Ar—OH), 9.25 (bs, 1H, Ar—OH); ¹³C-NMR ((CD₃)₂CO 125 MHz) δ 22.25 (2C, NCH(CH₃)₂), 51.24 (1C, NCH(CH₃)₂), 116.03 (2C, Ar—C), 116.20 (1C, Ar—C), 127.59 (1C, Ar—CSO₂), 130.45 (2C, Ar—C), 133.57 (1C, Ar—CNCH(CH₃)₂), 134.56 (2C, Ar—C), 158.34 (1C, Ar—COH), 161.72 (1C, Ar—COH); LRMS m/z 307.1 (M⁺); HRMS (C₁₅H₁₇NO₄S) calcd 307.0878 found 307.0877; Anal (C₁₅H₁₇NO₄S.0.3H₂O) C, H, N calcd 57.60% C, 5.67% H, 4.48% N found 57.50% C, 5.51% H, 4.29% N.

### N-sec-Butyl-4-hydroxy-N-(4-hydroxy-phenyl)-benzenesulfonamide (20h)

Following general procedure E, 9h was demethylated to yield 20h as a white solid. Isolated yield 56%. ¹H-NMR ((CD₃)₂CO 500 MHz) δ 0.91 (t, J=7.29 Hz, 3H, NCHCH₃(CH₂CH₃)), 0.97 (d, J=6.65 Hz, 3H, NCHCH₃(CH₂CH₃)), 1.20 (dp., J=13.72, 7.29 Hz, 1H, NCHCH₃(CH₂CH₃)), 1.32 (dp., J=13.94, 7.29 Hz, 1H, NCHCH₃(CH₂CH₃)), 4.21 (sextet, J=6.86 Hz, 1H, NCHCH₃(CH₂CH₃)), 6.78 (AA′XX′, 2H, Ar—H), 6.85 (AA′XX′, 2H, Ar—H), 6.95 (AA′XX′, 2H, Ar—H), 7.57 (AA′XX′, 2H, Ar—H), 8.61 (bs, 1H, Ar—OH), 9.25 (bs, 1H, Ar—OH); ¹³C-NMR ((CD₃)₂CO 125 MHz) δ 11.70 (1C, NCHCH₃(CH₂CH₃)), 20.14 (1C, NCHCH₃(CH₂CH₃)), 29.12 (1C, NCHCH₃(CH₂CH₃)), 57.42 (1C, NCHCH₃(CH₂CH₃)), 115.98 (2C, Ar—C), 116.10 (2C, Ar—C), 127.58 (1C, Ar—CSO₂), 130.42 (2C, Ar—C), 133.52 (1C, Ar—CN(s-Bu)), 134.34 (2C, Ar—C), 158.26 (1C, Ar—COH), 161.65 (1C, Ar—COH); LRMS m/z 321.1 (M⁺); HRMS (C₁₆H₁₉NO₄S) calcd 321.1035 found 321.1038; Anal (C₁₆H₁₉NO₄S.0.1H₂O) C, H, N calcd 59.46% C, 5.99% H, 4.33% N found 59.18% C, 5.68% H, 4.33% N.

### 4-Hydroxy-N-(4-hydroxy-phenyl)-N-(1-methyl-butyl)-benzenesulfonamide (20i)

Following general procedure E, 9i was demethylated to yield 20i as a white solid. Isolated yield 68%. ¹H-NMR ((CD₃)₂CO 500 MHz) δ 0.87 (t, J=7.18 Hz, NCHCH₃(CH₂CH₂CH₃)), 0.97 (d, J=6.65 Hz, 3H, NCHCH₃(CH₂CH₂CH₃)), 1.27 (m, 4H, NCHCH₃(CH₂CH₂CH₃)), 4.33 (m, 1H, NCHCH₃(CH₂CH₂CH₃)), 6.78 (AA′XX′, 2H, Ar—H), 6.84 (AA′XX′, 2H, Ar—H), 6.95 (AA′XX′, 2H, Ar—H), 7.56 (AA′XX′, 2H, Ar—H), 8.66 (bs, 1H, Ar—OH), 9.14 (bs, 1H, Ar—OH); ¹³C-NMR ((CD₃)₂CO 125 MHz) δ 14.05 (1C, NCHCH₃(CH₂CH₂CH₃)), 20.40 (1C, NCHCH₃(CH₂CH₂CH₃)), 20.44 (1C, NCHCH₃(CH₂CH₂CH₃)), 38.38 (1C, NCHCH₃(CH₂CH₂CH₃)), 55.32 (1C, NCHCH₃(CH₂CH₂CH₃)), 115.98 (2C, Ar—C), 116.11 (2C, Ar—C), 127.56 (1C, Ar—CSO₂), 130.45 (2C, Ar—C), 133.50 (1C, Ar—CN(2-Pentyl)), 134.39 (2C, Ar—C), 158.28 (1C, Ar—COH), 161.68 (1C, Ar—COH), LRMS m/z 335.2 (M⁺); HRMS (C₁₇H₂₁NO₄S) calcd 335.1191 found 335.1188; Anal (C₁₇H₂₁NO₄S.0.5H₂O) C, H, N calcd 59.28% C, 6.44% H, 4.07% N found 59.06% C, 6.15% H, 4.02% N.

### N-(1-Ethyl-propyl)-4-hydroxy-N-(4-hydroxy-phenyl)-benzenesulfonamide (20j)

Following general procedure E, 9j was demethylated to yield 20j as a white solid. Isolated yield 72%. ¹H-NMR ((CD₃)₂CO 500 MHz) δ 0.93 (t, J=7.40 Hz, 6H, NCH(CH₂CH₃)₂), 1.22 (m, 2H, NCH(CH₂CH₃)₂), 1.32 (m, 2H, NCH(CH₂CH₃)₂), 3.96 (m, 1H, NCH(CH₂CH₃)₂), 6.78 (AA′XX′, 2H, Ar—H), 6.85 (AA′XX′, 2H, Ar—H), 6.93 (AA′XX′, 2H, Ar—H), 7.52 (AA′XX′, 2H, Ar—H), 8.61 (bs, 1H, Ar—OH), 9.24 (bs, 1H, Ar—OH); ¹³C-NMR ((CD₃)₂CO 125 MHz) δ 11.98 (2C, NCH(CH₂CH₃)₂), 26.97 (2C, NCH(CH₂CH₃)₂), 64.34 (1C, NCH(CH₂CH₃)₂), 115.97 (2C, Ar—C), 116.02 (2C, Ar—C), 127.66 (1C, Ar—CSO₂), 130.50 (2C, Ar—C), 133.50 (1C, Ar—CN(3-Pentyl), 134.21 (2C, Ar—C), 158.26 (1C, Ar—COH), 161.62 (1C, Ar—COH); LRMS m/z 335.1 (M⁺); HRMS (C₁₇H₂₁NO₄S) calcd 335.1191 found 335.1198; Anal (C₁₇H₂₁NO₄S) C, H, N calcd 60.87% C, 6.31% H, 4.18% N found 60.48% C, 6.15% H, 4.18% N.

### 4-Hydroxy-N-(4-hydroxy-phenyl)-N-(2,2,2-trifluoro-ethyl)-benzenesulfonamide (20k)

Following general procedure E, 19a was demethylated to yield 20k as a white solid, mp=172-173° C. Isolated yield 62%. ¹H-NMR ((CD₃)₂CO 500 MHz) 64.36 (q, J=8.72 Hz, 2H, NCH₂CF₃), 6.77 (AA′XX′, 2H, Ar—H), 6.94 (m, 4H, Ar—H), 7.48 (AA′XX′, 2H, Ar—H), 8.73 (bs, 1H, Ar—OH), 9.30 (bs, 1H, Ar—OH); ¹³C-NMR ((CD₃)₂CO 125 MHz) δ 52.18 (q, J=34.06 (1C, NCH₂CF₃), 115.74 (2C, Ar—C), 115.79 (2C, Ar—C), 124.77 (q, J=279.87, 1C, NCH₂CF₃), 129.26 (1C, Ar—CSO₂), 130.37 (2C, Ar—C), 130.73 (2C, Ar—C), 131.41 (1C, Ar—CNCH₂CF₃), 157.66 (1C, Ar—COH), 161.90 (1C, Ar—COH); ¹⁹F-NMR ((CD₃)₂CO 470 MHz) δ −71.77 (3F, NCH₂CF₃); LRMS m/z 347.0 (M⁺); HRMS (C₁₄H₁₂F₃NO₄S) calcd 347.0439 found 347.0436; Anal (C₁₄H₁₂F₃NO₄S) C, H, N calcd 48.41% C, 3.51% H, 4.03% N found 48.12% C, 3.51% H, 3.81% N.

### 4-Hydroxy-N-(4-hydroxy-phenyl)-N-(3,3,3-trifluoro-propyl)benzenesulfonamide (20l)

Following general procedure E, 19b was demethylated to yield 20l as a white solid. Isolated yield 43%. ¹H-NMR ((CD₃)₂CO 500 MHZ) δ 2.41 (m, 2H, NCH₂CH₂CF₃), 3.79 (t, J=7.18 Hz, 2H, NCH₂CH₂CF₃), 6.79 (AA′XX′, 2H, Ar—H), 6.90 (AA′XX′, 2H, Ar—H), 6.96 (AA′XX′, 2H, Ar—H), 7.45 (AA′XX′, 2H, Ar—H), 8.65 (bs, 1H, Ar—OH), 9.36 (bs, 1H, Ar—OH); ¹³C-NMR ((CD₃)₂CO 125 MHz) δ 33.61 (q, J=27.62, 1C, NCH₂CH₂CF₃), 45.13 (q, J=3.68 Hz, 1C, NCH₂CH₂CF₃), 116.28 (2C, Ar—C), 116.39 (2C, Ar—C), 127.27 (1C, Ar—CSO₂), 130.91 (2C, Ar—C), 131.03 (2C, Ar—C), 131.27 (1C, Ar—CNCH₂CH₂CF₃), 158.08 (1C, Ar—COH), 162.26 (1C, Ar—COH); ¹⁹F-NMR ((CD₃)₂CO 470 MHz) δ −65.98 (3F, NCH₂CH₂CF₃); LRMS m/z 361.1 (M⁺); HRMS (C₁₅H₁₄F₃NO₄S) calcd 361.0596 found 361.0589; Anal (C₁₄H₁₂F₃NO₄S.0.5H₂O) C, H, N calcd 48.65% C, 4.08% H, 3.78% N found 48.33% C, 3.88% H, 3.61% N.

### N-(3-Fluoro-4-methoxy-phenyl)-4-methoxy-benzenesulfonamide (22a)

Following general procedure D, 21a was reacted with 18 to yield 22a as a white solid. Isolated yield 86%. ¹H-NMR ((CD₃)₂CO 500 MHz) δ3.78 (s, 3H, Ar—OCH₃), 3.81 (s, 3H, Ar—OCH₃), 6.92 (ddd, J=8.79, 2.57, 1.29 Hz, 1H, Ar—H), 6.96 (d, J=9.00 Hz, 1H, Ar—H), 7.00 (AA′XX′, 2H, Ar—H), 7.03 (dd, J=12.65, 2.57 Hz, 1H, Ar—H), 7.69 (AA′XX′, 2H, Ar—H), 8.73 (bs, 1H, A-NHSO₂), ¹³C-NMR ((CD₃)₂CO 125 MHz) δ 55.94 (1C, Ar—OCH₃), 56.46 (1C, Ar—OCH₃), 111.09 (d, J=21.17 Hz, 1C, Ar—C), 114.62 (d, J=2.76 Hz, 1C, Ar—C), 114.88 (2C, Ar—C), 118.70 (d, J=3.68 Hz, 1C, Ar—C), 130.04 (2C, Ar—C), 131.70 (d, J=9.21 Hz, 1C, Ar—CNH), 131.95 (1C, Ar—CSO₂), 145.87 (d, J=11.05 Hz, Ar—COCH₃), 152.58 (d, J=244.88 Hz, 1C, Ar—CF), 163.85 (1C, Ar—COCH₃); ¹⁹F-NMR ((CD₃)₂C) 470 MHz) δ −134.83 (1F, Ar—F); LRMS m/z 311.1 (M⁺); HRMS (C₁₄H₁₄FNO₄S) calcd 311.0628 found 311.0625.

### N-(3-Chloro-4-methoxy-phenyl)-4-methoxy-benzenesulfonamide (22b)

Following general procedure D, 21b was reacted with 18 to yield 22b as a white solid. Isolated yield 95%. ¹H-NMR ((CD₃)₂CO 500 MHz) δ 3.80 (s, 3H, Ar—OCH₃), 3.81 (s, 3H, Ar—OCH₃), 6.96 (d, J=8.79 Hz, 1H, Ar—H), 7.00 (AA′XX′, 2H, Ar—H), 7.10 (dd, J=8.79, 2.79 Hz, 1H, Ar—H), 7.24 (d, J=2.57 Hz, 1H, Ar—H), 7.68 (AA′XX′, 2H, Ar—H), 8.67 (bs, 1H, Ar—NHSO₂); ¹³C-NMR ((CD₃)₂CO 125 MHz) δ 55.95 (1C, Ar—OCH₃), 56.47 (1C, Ar—OCH₃), 113.43 (1C, Ar—C), 114.89 (2C, Ar—C), 122.61 (1C, Ar—CCl), 122.71 (1C, Ar—C), 124.76 (1C, Ar—C), 130.03 (2C, Ar—C), 131.91 (1C, Ar—CSO₂), 131.94 (1C, Ar—CNH), 153.40 (1C, Ar—C), 163.85 (1C, Ar—C); LRMS m/z 327.1 (M⁺); HRMS (C₁₄H₁₄ClNO₄S) calcd 327.0332 found 327.0325.

### N-(3-Fluoro-4-methoxy-phenyl)-4-methoxy-N-propyl-benzenesulfonamide (23a)

Following general procedure A, 22a was reacted with 8c to yield 23a as a white solid. Isolated yield 95%. ¹H-NMR (CDCl₃ 500 MHz) δ 0.87 (t, J=7.40 Hz, 3H, NCH₂CH₂CH₃), 1.39 (sextet, J=7.29 Hz, 2H, NCH₂CH₂CH₃), 3.40 (t, J=7.07 Hz, 2H, NCH₂CH₂CH₃), 3.85 (s, 3H, Ar—OCH₃), 3.86 (s, 3H, Ar—OCH₃), 6.72 (dd, J=12.01, 2.36 Hz, 1H, Ar—H), 6.81 (ddd, J=8.79, 2.36, 1.29 Hz, 1H, Ar—H), 6.86 (t, J=9.00 Hz, 1H, Ar—H), 6.91 (AA′XX″, 2H, Ar—H), 7.50 (AA′XX′, 2H, Ar—H); ¹³C-NMR (CDCl₃ 125 MHz) δ 11.06 (1C, NCH₂CH₂CH₃), 21.46 (1C, NCH₂CH₂CH₃), 52.32 (1C, NCH₂CH₂CH₃), 55.70 (1C, Ar—OCH₃), 56.34 (1C, Ar—OCH₃), 112.93 (d, J=1.84 Hz, 1C, Ar—C), 114.09 (2C, Ar—C), 116.61 (d, J=19.33 Hz, 1C, Ar—C), 129.77 (1C, Ar—CSO₂), 129.84 (2C, Ar—C), 131.90 (d, J=8.29 Hz, 1C, Ar—CN(n-Pr)), 147.46 (d, J=10.13 Hz, 1C, Ar—COCH₃), 151.77 (d, J=248.57 Hz, 1C, Ar—CF), 163.04 (1C, Ar—COCH₃); ¹⁹F-NMR (CDCl₃ 470 MHz) δ −133.66 (1F, Ar—F); LRMS m/z 353.2 (M⁺); HRMS (C₁₇H₂₀FNO₄S) calcd 353.1097 found 353.1093.

### N-(3-Chloro-4-methoxy-phenyl)-4-methoxy-N-propyl-benzenesulfonamide (23b)

Following general procedure A, 22b was reacted with 8c to yield 23b as a white solid. Isolated yield 85%. ¹H-NMR ((CD₃)₂CO 500 MHz) δ 0.87 (t, J=7.40 Hz, 3H, NCH₂CH₂CH₃), 1.38 (sextet, J=7.29 Hz, 2H, NCH₂CH₂CH₃), 3.49 (t, J=6.97 Hz, 2H, NCH₂CH₂CH₃), 3.88 (s, 3H, Ar—OCH₃), 3.89 (s, 3H, Ar—OCH₃), 6.99 (dd, J=8.79, 2.36 Hz, 1H, Ar—H), 7.05 (d, J=8.36 Hz, 1H, Ar—H), 7.06 (AA′XX′, 2H, Ar—H), 7.08 (d, J=2.57 Hz, 1H, Ar—H), 7.53 (AA′XX′, 2H, Ar—H); ¹³C-NMR ((CD₃)₂CO 125 MHz) δ 11.18 (1C, NCH₂CH₂CH₃), 22.00 (NCH₂CH₂CH₃), 52.68 (NCH₂CH₂CH₃), 56.07 (1C, Ar—OCH₃), 56.60 (1C, Ar—OCH₃), 112.86 (1C, Ar—C), 114.89 (2C, Ar—C), 122.27 (1C, Ar—CCl), 129.39 (1C, Ar—C), 130.55 (2C, Ar—C), 130.76 (1C, Ar—CSO₂), 131.16 (1C, Ar—C), 133.19 (1C, Ar—CN(n-Pr), 155.38 (1C, Ar—COCH₃), 163.95 (1C, Ar—COCH₃); LRMS m/z 369.1 (M⁺); HRMS (C₁₇H₂₀ClNO₄S) calcd 369.0811 found 369.0795.

### N-(3-Fluoro-4-hydroxy-phenyl)-4-hydroxy-N-propyl-benzenesulfonamide (24a)

Following general procedure E, 23a was demethylated to produce 24a as a white solid. Isolated yield 53%. ¹H-NMR ((CD₃)₂CO 500 MHz) δ 0.88 (t, J=7.40 Hz, 3H, NCH₂CH₂CH₃), 1.38 (sextet, J=7.29 Hz, 2H, NCH₂CH₂CH₃), 3.46 (t, J=6.97 Hz, 2H, NCH₂CH₂CH₃), 6.72 (ddd, J=8.58, 2.36,1.29 Hz, 1H, Ar—H), 6.82 (dd, J=12.01, 2.36 Hz, 1H, Ar—H), 6.91 (dd, J=9.65, 8.79 Hz, 1H, Ar—H), 6.95 (AA′XX′, 2H, Ar—H), 7.44 (AA′XX′, 2H, Ar—H), 9.10 (bs, 2H, Ar—OH); ¹³C-NMR ((CD₃)₂CO 125 MHz) δ 11.20 (1C, NCH₂CH₂CH₃), 21.98 (1C, NCH₂CH₂CH₃), 52.64 (1C, NCH₂CH₂CH₃), 116.23 (2C, Ar—C), 117.57 (d, J=19.33 Hz, 1C, Ar—C), 118.03 (d, J=3.68 Hz, 1C, Ar—C), 126.04 (d, J=2.76 Hz, 1C, Ar—C), 129.76 (1C, Ar—CSO₂), 130.79 (2C, Ar—C), 132.12 (d, J=7.36 Hz, 1C, Ar—CNCH₂CH₂CH₃), 145.13 (d, J=12.89 Hz, 1C, Ar—COH), 151.46 (d, J=242.12 Hz, 1C, Ar—CF), 162.11 (1C, Ar—COH); ¹⁹F-NMR ((CD₃)₂CO 470 MHz) δ −137.27 (1F, Ar—F); LRMS m/z 325.1 (M⁺); HRMS (C₁₅H₁₆FNO₄S) calcd 325.0784 found 325.0781.

### N-(3-Chloro-4-hydroxy-phenyl)-4-hydroxy-N-propyl-benzenesulfonamide (24b)

Following general procedure E, 23b was demethylated to produce 24b as a white solid. mp=138-140° C. Isolated yield 53%. ¹H-NMR ((CD₃)₂CO 500 MHz) δ 0.87 (t, J=7.40 Hz, 3H, NCH₂CH₂CH₃), 1.38 (sextet, J=7.29 Hz, 2H, NCH₂CH₂CH₃), 3.46 (t, J=7.07 Hz, 2H, NCH₂CH₂CH₃), 6.85 (dd, J=8.58, 2.57 Hz, 1H, Ar—H), 6.95 (m, 3H, Ar—H), 7.03 (d, J=2.57 Hz, 1H, Ar—H), 7.45 (AA′XX′, 2H, Ar—H), 9.17 (s, 2H, Ar—OH); ¹³C-NMR ((CD₃)₂CO 125 MHz) δ 11.19 (1C, NCH₂CH₂CH₃), 22.00 (1C, NCH₂CH₂CH₃), 52.68 (1C, NCH₂CH₂CH₃), 116.25 (2C, Ar—C), 117.19 (1C, Ar—C), 120.61 (1C, Ar—CCl), 129.41 (1C, Ar—C), 129.73 (1C, Ar—CSO₂), 130.80 (2C, Ar—C), 131.24 (1C, Ar—C), 132.71 (1C, Ar—CN(n-Pr)), 153.39 (1C, Ar—COCH₃), 162.15 (1C, Ar—COCH₃); LRMS m/z 341.1 (M⁺); HRMS (C₁₅H₁₆ClNO₄S) calcd 341.0489 found 341.0490.

### 3,3,3-Trifluoro-N-(3-fluoro-4-methoxy-phenyl)-propionamide (25a)

Following general procedure C, 21a was reacted with 15 to yield 25a as a white solid. Isolated yield 62%. ¹H-NMR ((CD₃)₂CO 500 MHz) δ 3.42 (q, J=10.79 Hz, 2H, NC═OCH₂CF₃), 3.83 (s, 3H, Ar—OCH₃), 7.05 (t, J=9.11 Hz, 1H, Ar—H), 7.25 (ddd, J=8.79, 2.36, 1.72 Hz, 1H, Ar—H), 7.59 (dd, J=13.29, 2.36 Hz, 1H, Ar—H), 9.53 (bs, 1H, Ar—NHC═O); ¹³C-NMR ((CD₃)₂CO 125 MHz) δ 41.78 (q, J=28.54 Hz, 1C, NC═OCH₂CF₃), 109.12 (d, J=23.02 Hz, 1C, Ar—C), 114.64 (d, J=2.76 Hz, 1C, Ar—C), 116.24 (d, J=3.68 Hz, 1C, Ar—C), 125.67 (q, J=276.19 Hz, 1C, NC═OCH₂CF₃), 132.75 (d, J=9.21 Hz, 1C, Ar—CNHC═O), 145.09 (d, J=11.05 Hz, 1C, Ar—COCH₃), 152.51 (d, J=243.04 Hz, 1C, Ar—CF), 162.05 (q, J=3.68 Hz, 1C, NC═OCH₂CF₃); ¹⁹F-NMR ((CD₃)₂CO 470 MHz) δ −63.91 (3F, NC═OCH₂CF₃), −135.13 (1F, Ar—F); LRMS m/z 251.1 (M⁺); HRMS (C₁₀H₉F₄NO₂) calcd 251.0569 found 251.0569.

### 3,3,3-Trifluoro-N-(3-chloro-4-methoxy-phenyl)-propionamide (25b)

Following general procedure C, 21b was reacted with 15 to yield 25b as a white solid. ¹H-NMR ((CD₃)₂CO 500 MHz) δ 3.43 (q, J=10.79 Hz, 2H, NC═OCH₂CF₃), 3.84 (s, 3H, Ar—OCH₃), 7.03 (d, J=9.00 Hz, 1H, Ar—H), 7.44 (dd, J=9.00, 2.57 Hz, 1H, Ar—H), 7.78 (d, J=2.57 Hz, 1H, Ar—H); ¹³C-NMR ((CD₃)₂CO 125 MHz) δ 41.73 (q, J=28.54 Hz, 1C, NC═OCH₂CF₃), 56.53 (1C, Ar—OCH₃), 113.32 (1C, Ar—C), 120.21 (1C, Ar—C), 122.46 (1C, Ar—CCl), 122.49 (1C, Ar—C), 125.55 (q, J=276.19 Hz, 1C, NC═OCH₂CF₃), 132.88 (1C, Ar—CNC═O), 152.62 (1C, Ar—COCH₃), 162.04 (q, J=3.68 Hz, 1C, NC═OCH₂CF₃); ¹⁹F-NMR ((CD₃)₂CO 470 MHz) δ −63.87 (3F, NC═OCH₂CF₃), LRMS m/z 267.1 (M⁺); HRMS (C₁₀H₉ClF₃NO₂) calcd 267.0274 found 267.0270.

### (3-Fluoro-4-methoxy-phenyl)-(3,3,3-trifluoro-propyl)-amine (26a)

Following general procedure B, 25a was reduced to yield 26a as a colorless oil. Isolated yield 85%. ¹H-NMR (CDCl₃ 500 MHz) δ 2.38 (qt, J=10.72, 6.86 Hz, 2H, NCH₂CH₂CF₃), 3.37 (t, J=6.97 Hz, 2H, NCH₂CH₂CF₃), 3.81 (s, 3H, Ar—OCH₃), 6.31 (ddd, J=8.58, 2.79, 1.29 Hz, 1H, Ar—H), 6.41 (dd, J=13.29, 2.79 Hz), 6.85 (t, J=9.11 Hz, 1H, Ar—H); ¹³C-NMR (CDCl₃ 125 MHz) δ 33.41 (q, J=27.62 Hz, NCH₂CH₂CF₃), 37.84 (q, J=3.68 Hz, NCH₂CH₂CF₃), 102.26 (d, J=22.09 Hz, 1C, Ar—C), 108.24 (d, J=3.68 Hz, 1C, Ar—C), 116.11 (d, J=2.76 Hz, 1C, Ar—C), 126.64 (q, J=277.11 Hz, 1C, Ar—CF), 140.12 (d, J=11.05 Hz, 1C, Ar—CNCH₂CH₂CF₃), 142.17 (d, J=9.21 Hz, 1C, Ar—COCH₃), 153.70 (d, J=243.96 Hz, 1H, Ar—CF); ¹⁹F-NMR (CDCl₃ 470 MHz) δ −65.44 (3F, NCH₂CH₂CF₃), −133.79 (1F, Ar—F); LRMS m/z 237.1 (M⁺); HRMS (C₁₇H₂₀F₄NO) calcd 237.0777 found 237.0771.

### (3-Chloro-4-methoxy-phenyl)-(3,3,3-trifluoro-propyl)-amine (26b)

Following general procedure B, 25b was reduced to yield 26b as a colorless oil. Isolated yield 93%. ¹H-NMR ((CD₃)₂CO 500 MHz) δ 2.51 (qt, J=11.15, 7.29 Hz, 2H, NCH₂CH₂CF₃), 3.38 (t, J=7.07 Hz, 2H, NCH₂CH₂CF₃), 3.76 (s, 3H, Ar—OCH₃) 6.59 (dd, J=8.79, 2.14 Hz, 1H, Ar—H), 6.74 (d, J=2.14 Hz, 1H, Ar—H), 6.91 (d, J=8.79 Hz, 1H, Ar—H); ¹³C-NMR ((CD₃)₂CO 125 MHz) δ 33.71 (q, J=26.70 Hz, 1C, NCH₂CH₂CF₃), 37.98 (q, J=2.76 Hz, 1C, NCH₂CH₂CF₃), 57.03 (1C, Ar—OCH₃), 112.66 (1C, Ar—C), 115.27 (1C, Ar—C), 115.38 (1C, Ar—C), 123.76 (1C, Ar—CCl), 127.89 (q, J=276.19 Hz, 1C, NCH₂CH₂CF₃), 143.94 (1C, Ar—CNCH₂CH₂CF₃), 148.01 (1C, Ar—COCH₃); ¹⁹F-NMR ((CD₃)₂CO 470 MHz) δ −66.07 (3F, NCH₂CH₂CF₃); LRMS m/z 253.1 (M⁺); HRMS (C₁₀H₁₁ClF₃NO) calcd 253.0481 found 253.0475.

### N-(3-Fluoro-4-methoxy-phenyl)-4-methoxy-N-(3,3,3-trifluoro-propyl)-benzenesulfonamide (27a)

Following general procedure D, 26a was reacted with 18 to yield 27 as a white solid. Isolated yield 76%. ¹H-NMR ((CD₃)₂CO 500 MHz) δ 2.46 (qt, J=10.93. 7.29 Hz, 2H, NCH₂CH₂CF₃), 3.86 (t, J=7.18 Hz, 2H, NCH₂CH₂CF₃), 3.88 (s, 3H, Ar—OCH₃), 3.88 (s, 3H, Ar—OCH₃), 6.90 (ddd, J=8.79, 2.57, 1.50 Hz, 1H, Ar—H), 6.93 (dd, J=13.72, 2.57 Hz, 1H, Ar—H), 7.08 (m, 3H, Ar—H), 7.57 (AA′XX′, 2H, Ar—H); ¹³C-NMR ((CD₃)₂CO 125 MHz) δ 33.54 (q, J=27.62 Hz, 1C, NCH₂CH₂CF₃), 45.07 (q, J=3.68 Hz, 1C, NCH₂CH₂CF₃), 113.97 (d, J=1.84 Hz, 1C, Ar—C), 115.05 (2C, Ar—C), 117.46 (d, J=19.33, 1C, Ar—C), 126.13 (d, J=3.68 Hz, 1C, Ar—C), 129.40 (q, J=276.19 Hz, 1C, NCH₂CH₂CF₃), 130.03 (1C, Ar—CSO₂), 130.69 (2C, Ar—C), 132.17 (d, J=8.29 Hz, 1C, Ar—CNCH₂CH₂CF₃), 148.56 (d, J=11.05 Hz, 1C, Ar—COCH₃), 152.29 (d, J=246.73 Hz, 1C, Ar—C), 164.25 (1C, Ar—COCH₃); ¹⁹F-NMR ((CD₃)₂CO 470 MHz) δ −65.86 (3F, NCH₂CH₂CF₃), −134.69 (1F, Ar—F); LRMS m/z 407.2 (M⁺); HRMS (C₁₇H₁₈F₄NO₄S) calcd 407.0814 found 407.0806.

### N-(3-Chloro-4-methoxy-phenyl)-4-methoxy-N-(3,3,3-trifluoro-propyl)-benzenesulfonamide (27b)

Following general procedure D, 26b was reacted with 18 to yield 27b. Isolated yield 89%. ¹H-NMR (CDCl₃ 500 MHz) δ 2.33 (m, 2H, NCH₂CH₂CF₃), 3.68 (t, J=7.50 Hz, 2H, NCH₂CH₂CF₃), 3.85 (s, 3H, Ar—OCH₃), 3.88 (s, 3H, Ar—OCH₃), 6.85 (d, J=8.79 Hz, 1H, Ar—H), 6.93 (m, 3H, Ar—H), 7.00 (d, J=2.57 Hz, 1H, Ar—H), 7.50 (AA′XX′, 2H, Ar—H); ¹³C-NMR (CDCl₃ 125 MHz) δ 33.59 (q, J=28.54 Hz, 1C, NCH₂CH₂CF₃), 44.65 (q, J=3.68 Hz, 1C, NCH₂CH₂CF₃), 55.75 (1C, Ar—OCH₃), 56.38 (1C, Ar—OCH₃), 112.07 (1C, Ar—C), 114.31 (2C, Ar—C), 122.71 (1C, Ar—CCl), 125.77 (q, J=277.11 Hz, 1C, NCH₂CH₂CF₃), 128.62 (1C, Ar—C), 128.83 (1C, Ar—CSO₂), 129.97 (2C, Ar—C), 130.48 (1C, Ar—C), 131.72 (1C, Ar—CNCH₂CH₂CF₃), 155.16 (1C, Ar—COCH₃), 163.46 (1C, Ar—COCH₃); ¹⁹F-NMR (CDCl₃ 470 MHz) δ −65.56 (3F, NCH₂CH₂CF₃); LRMS m/z 423.0 (M⁺); HRMS (C₁₇H₁₇ClF₃NO₄S) calcd 423.0519 found 423.0513.

### N-(3-Fluoro-4-hydroxy-phenyl)-4-hydroxy-N-(3,3,3-trifluoro-propyl)-benzenesulfonamide (28a)

Following general procedure E, 27a was demethylated to yield 28a as a white solid. Isolated yield 54%. ¹H-NMR ((CD₃)₂CO 500 MHz) δ 2.45 (qt, J=10.93, 7.07 Hz, 2H, NCH₂CH₂CF₃), 3.82 (t, J=7.18 Hz, 2H, NCH₂CH₂CF₃), 6.76 (ddd, J=8.79, 2.57, 1.29 Hz, 1H, Ar—H), 6.89 (dd, J=11.79, 1.29 Hz), 6.94 (t, J=9.65 Hz, 1H, Ar—H), 6.97 (AA′XX′, 2H, Ar—H), 7.48 (AA′XX′, 2H, Ar—H), 9.19 (bs, 2H, Ar—OH); ¹³C-NMR ((CD₃)₂CO 125 MHz) δ 33.56 (q, J=27.62 Hz, 1C, NCH₂CH₂CF₃), 45.04 (q, J=3.68 Hz, 1C, NCH₂CH₂CF₃), 116.41 (2C, Ar—C), 117.82 (d, J=19.33 Hz, 1C, Ar—C), 118.26 (d, J=2.76 Hz, 1C, Ar—C), 126.19 (d, J=3.68 Hz, 1C, Ar—C), 127.24 (q, J=276.19 Hz, 1C, NCH₂CH₂CF₃), 129.02 (1C, Ar—CSO₂), 130.96 (2C, Ar—C), 131.53 (d, J=8.29 Hz, 1C, Ar—CNCH₂CH₂CF₃), 145.79 (d, J=12.89 Hz, 1C, Ar—COH), 151.52 (d, J=243.04 Hz, 1C, Ar—CF), 162.46 (1C, Ar—COH); ¹⁹F-NMR ((CD₃)₂CO 470 MHz) δ −65.97 (3F, NCH₂CH₂CF₃), −136.79 (1F, Ar—F); LRMS m/z 379.0 (M⁺); HRMS (C₁₅H₁₃F₄NO₄S) calcd 379.0501 found 379.0507.

### N-(3-Chloro-4-hydroxy-phenyl)-4-hydroxy-N-(3,3,3-trifluoro-propyl)-benzenesulfonamide (28b)

Following general procedure E, 27b was demethylated to produce 28b as a white solid. Isolated yield 46%. 1H-NMR ((CD3)2CO 500 MHz) δ 2.46 (qt, J=10.93, 7.07 Hz, 2H, NCH2CH2CF3), 3.82 (t, J=7.07 Hz, 2H, NCH2CH2CF3), 6.89 (dd, J=8.58, 2.57 Hz, 1H, Ar—H), 6.97 (m, 3H, Ar—H), 7.09 (d, J=2.57 Hz, 1H, Ar—H), 7.48 (AA′XX′, 2H, Ar—H), 9.14 (bs, 1H, Ar—OH), 9.41 (bs, 1H, Ar—OH); 13C-NMR ((CD3)2CO 125 MHz) δ 33.60 (q, J=27.62 Hz, 1C, NCH2CH2CF3), 45.11 (q, J=3.68 Hz, 1C, NCH2CH2CF3), 116.42 (2C, Ar—C), 117.38 (1C, Ar—C), 120.86 (1C, Ar—CCl), 127.27 (q, J=277.11 Hz, 1C, NCH2CH2CF3), 129.02 (1C, Ar—CSO2), 129.54 (1C, Ar—C), 130.98 (2C, Ar—C), 131.49 (1C, Ar—C), 132.18 (1C, Ar—CNCH2CH2CF3), 153.82 (1C, Ar—COH), 162.48 (1C, Ar—COH); 19F-NMR ((CD3)2CO 470 MHz) □ −65.96 (3F, NCH2CH2CF3); LRMS m/z 395.1 (M⁺); HRMS (C18H13ClF3NO4S) calcd 395.0206 found 395.0212.

### Example 2

### Estrogen Receptor Binding Affinity Assays

Relative binding affinities reported herein were determined by a competitive radiometric binding assay as previously described (Carlson, K. E.; Choi, I.; Gee, A.; Katzenellenbogen, B. S.; Katzenellenbogen, J. A. Altered ligand binding properties and enhanced stability of a constitutively active estrogen receptor: evidence that an open pocket conformation is required for ligand interaction. Biochemistry 1997, 36, 14897-14905; Katzenellenbogen, J. A.; Johnson, H. J., Jr.; Myers, H. N. Photoaffinity labels for estrogen binding proteins of rat uterus. Biochemistry 1973, 12, 4085-4092.) [3H]estradiol (10 nM) was used as tracer ([6,7-3H]estra-1,3,5,(10)triene-3,17-β-diol, 51-53 Ci/mmol, Amersham Biosciences, Piscataway, N.J.), and purified full-length human ERalpha and ERbeta receptors purchased from Pan Vera (Madison, Wis.). Incubations were for 18-24 h at 0° C. Hydroxyapatite (BioRad, Hercules, Calif.) was used to absorb the receptor-ligand complexes and free ligand was washed away. The binding affinities are expressed as relative binding affinity (RBA) values with the RBA of estradiol set to 100%. The values given are the average±range or SD of two to three independent determinations. Estradiol binds to ERalpha with a K_{d} of 0.2 nM and to ERbeta with a K_{d} of 0.5 nM.

### Example 3

### Cell Culture and Transient Transfections

Human endometrial cancer (HEC-1) cells were maintained in minimum essential medium (MEM) plus phenol red supplemented with 5% calf serum and 5% fetal calf serum. Cells were plated in phenol-red-free improved MEM and 5% charcoal dextran-treated calf serum (CDCS) and were given fresh medium 24 h before transfection. Transfection assays were performed in 24 well plates using a mixture of 0.35 mL of serum-free improved MEM medium and 0.15 mL of Hank's balanced salt solution containing 5 μL of Lipofectin (Trademark, Life Technologies, Inc., Gaithersburg, Md.), 1.6 microg of Transferrin (Trademark, Sigma, St. Louis, Mo.), 0.5 microg of pCMV β-galactosidase as internal control, 1 μg of 2ERE-pS2-Luc, and 100 ng of ER expression vector per well. The cells were incubated at 37° C. in a 5% CO₂-containing incubator for 5 h. The medium was then replaced with fresh improved MEM supplemented with 5% CDCS plus the desired concentrations of ligand. Cells were harvested 24 h later. Luciferase and beta-galactosidase activity were assayed as described (McInerney, E. M.; Tsai, M. J.; O'Malley, B. W.; Katzenellenbogen, B. S. Analysis of estrogen receptor transcriptional enhancement by a nuclear hormone receptor coactivator. Proc Natl. Acad. Sci. U S A 1996, 93, 10069-10073.)

Transient transfections were also carried out in U2-OS cells (FIGS. 3A-B) following a similar procedure.

### Example 4

### Assessment of Expression of Endogenous Genes Using Quantitative PCR (Polymerase Chain Reaction)Methods

Dose-response in U2-OS cells which stably express ER alpha or ER beta was assessed employing quantitative PCR essentially as described in Stossi F, Barnett D H, Frasor J, Komm B, Lyttle C R, Katzenellenbogen B S “Transcriptional profiling of estrogen-regulated gene expression via estrogen receptor ER alpha or ER beta in human osteosarcoma cells; distinct and common target genes for these receptors,” (2004) Endocrinology 145:3473-3486 employing reporter constructs as described in Example 3.

More specifically real-time PCR was carried out on the indicated genes to evaluate mRNA levels of ER alpha or ER beta in U2-OS stably transfected cells. The primers used are listed in Table 1 of Stossi et al. supra. One microgram of total RNA from each sample was reverse transcribed in a total volume of 20 μl using 200 U reverse transcriptase, 50 pmol random hexamers, and 1 mM deoxynucleotide triphosphates (New England Biolabs, Beverly, Mass.). The resulting cDNA was then diluted to a total volume of 100 μl. Each real-time PCR consisted of 5 μl of diluted reverse transcription product, 1× SYBR Green PCR Master Mix (Applied Biosystems, Foster City, Calif.), and 50 nM of forward and reverse primers. Reactions were carried out in an ABI Prism 7700 Sequence Detection System (Applied Biosystems) for 40 cycles (95 C for 15 sec, 60 C for 1 min) after an initial 10-min incubation at 95 C. The FC in expression was calculated using the DDCt comparative threshold cycle method with the ribosomal protein 36B4 mRNA as an internal control. Gene expression is normalized to an endogenous reference gene (36B4) and the FC in gene expression is then determined relative to the vehicle-treated control. Further details are given in Stossi et al. supra.

U2-OS cells expressing either ER alpha or ER beta were transfected with 2x-pS2-ERE-Luc reporter gene and beta-galalactosidase (as an internal control gene) and then treated with ligands as indicated in FIGS. 3A-D for 24 hours before assessing luciferase activity. Values are expressed as % of E2 activity at 1 nM.

### Example 5

### Molecular Modeling

Small-molecule geometry optimization and modeling of ligand-protein complexes were carried out in Sybyl (version 6.7, Tripos). For ER alpha, the estradiol-ER alpha ligand binding domain (1ERE) crystal structure was used. For ER beta, the genistein-ER beta ligand binding domain (1QKM) crystal structure was used. The ligand 24c was pre-positioned by overlaying a p-hydroxyphenyl ring with the A-ring of estradiol or genistein. Estradiol or genistein was then deleted and ligand 24c was merged into its place. The rotatable bonds of ligand 24c were set, and the 24c was then allowed to reposition itself in the binding pocket while the protein remained fixed. The best docked ligand-receptor complexes were then subjected to a three-part minimization process: In the first step, the torsional bonds were minimized using the torsmin command. In the second, step the ligand 24c and amino acids within 8 Å of the ligand were minimized while holding the protein backbone fixed. In the final step, the ligand-receptor complex was minimized with the anneal command, utilizing a hot radius of 8 Å and an interesting radius of 16 Å. All minimizations used the MMFF94 force field with the Powell gradient (final rms <0.1 kcal mol-1 Å-1).

We claim:

1. A method for selectively regulating the expression of one or more genes in a mammalian cell or in mammalian tissue, the expression of which are affected by an estrogen receptor (ER) which comprises the step of contacting the cell or tissue with an amount or combined amount of one or more compounds of formula I or salts, stereoisomers or prodrugs thereof sufficient to affect the expression of one or more genes in one or more cells or tissues wherein formula I is 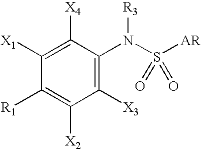
or a salt, stereoisomer or prodrug thereof wherein:
AR is an optionally substituted aryl group;
R₃ is an alkyl, alkenyl, alkynyl, benzyl, or phenyl group;
R₁ is a hydrogen, a halide, a hydroxy, thiol, an alkyl, alkenyl, alkynyl, benzyl, phenyl alkoxy, thioalkoxy, or aryloxy group; and
X₁—X₄, independently of one another, are selected from the group consisting of hydrogens, halogens, alkyl groups, alkoxy groups, —CO—R groups, —SR groups, cyano groups, nitro groups, hydroxy groups, alkoxy groups, thiol groups, and thioalkoxy groups, where R is H, or an alkyl group, wherein R₃ can be linked with X₃, or X₄ to form a 5, 6 or 7-member ring which may be an aromatic ring, or may contain one or two double bonds and wherein the ring optionally contains one or two additional heteroatoms wherein all alkyl, alkenyl, alkynyl, aryl, benzyl and phenyl groups are optional substituted and wherein optional substitution means substitution with one or more halogens, cyano groups, nitro groups, hydroxy groups, alkoxy groups, thiol groups, thioalkoxy groups, aryloxy groups, N(R)′₂ groups, CON(R′)₂ groups or —COOR′ groups, where R′ is H or an alkyl group and where R′ groups may be linked to form a cyclic alkyl group.

2. The method of claim 1 wherein AR is an optionally substituted phenyl group: 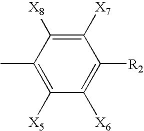
or
an optionally substituted thiophene or furan group: 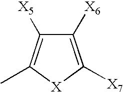
where X is S or O;
R₂ is hydrogen, an OR group, a halogen, an alkyl group, an alkoxy group, —CO—R group, —SR group, a cyano group, a nitro group, a thiol group, and a hydroxy group, where R is H, or an alkyl group; and
X₅—X₈, independently of one another, are selected from the group consisting of hydrogens, halogens, alkyl groups, alkoxy groups, thioalkoxyl groups, —CO—R groups, cyano groups, nitro groups, thiol groups, and hydroxy groups, where R is H, or a alkyl group.

3. The method of any one of claims 1 or 2 wherein R₁ is OH, an alkoxy group or an aryl group.

4. The method of claim 1 wherein AR is an optionally substituted phenyl group.

5. The method of claim 2 wherein R₂ and R₁, independently, are hydrogen or an OR group; X₁—X₈ are hydrogens or halogens; and R₃ is selected from the group consisting of C1-C6 alkyl or C2-C6 alkenyl groups which are optionally substituted with one or more halogens, cyano groups, or nitro groups.

6. The method of claim 1 wherein R₃ is a fluorinated alkyl group.

7. The method of claim 1 wherein R₃ contains a trifluoromethyl group.

8. The method of claim 1 wherein R₃ is a methyl, ethyl or propyl group that is optionally substituted with one or more halogens.

9. The method of claim 2 wherein two of X₁—X₈ are halogens

10. The method of claim 2 wherein two of X₁—X₈ are fluorines.

11. The method of claim 1 wherein the compound, salt, stereoisomer or prodrug of formula I exhibits minimal effect on the expression of a gene in the cell or tissue the expression of which is regulated through ER alpha.

12. The method of claim 1 wherein the compound, salt, stereoisomer, or prodrug of formula I exhibits a Relative Binding Affinity (ER beta/ER alpha) of 10 or more.

13. The method of claim 1 wherein the compound, salt or prodrug of formula I exhibits a Relative Binding Affinity (ER beta/ER alpha) of 25 or more.

14. A method for treating a disease, a disorder, a condition or symptoms affected by an estrogen receptor by ER beta wherein an amount or combined amount of one or more of the compounds, salts, stereoisomers or prodrugs of formula I is administered to a mammal in need of such treatment in an amount effective to affect expression of one or more genes the expression of which is regulated by ER beta wherein formula I is 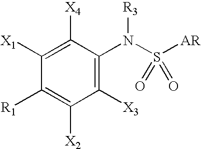
wherein:
AR is an optionally substituted aryl group;
R₃ is an alkyl, alkenyl, alkynyl, benzyl, or phenyl group;
R₁ is a hydrogen, a halide, a hydroxy, thiol, an alkyl, alkenyl, alkynyl, benzyl, phenyl alkoxy, thioalkoxy, or aryloxy group; and
X₁—X₄, independently of one another, are selected from the group consisting of hydrogens, halogens, alkyl groups, alkoxy groups, —CO—R groups, —SR groups, cyano groups, nitro groups, hydroxy groups, alkoxy groups, thiol groups, and thioalkoxy groups, where R is H, or an alkyl group, wherein R₃ can be linked with X₃, or X₄ to form a 5, 6 or 7-member ring which may be an aromatic ring, or may contain one or two double bonds and wherein the ring optionally contains one or two additional heteroatoms wherein all alkyl, alkenyl, alkynyl, aryl, benzyl and phenyl groups are optional substituted and wherein optional substitution means substitution with one or more halogens, cyano groups, nitro groups, hydroxy groups, alkoxy groups, thiol groups, thioalkoxy groups, aryloxy groups, N(R)′₂ groups, CON(R′)₂ groups or —COOR′ groups, where R′ is H or an alkyl group and where R′ groups may be linked to form a cyclic alkyl group.

15. The method of claim 14 wherein in the compound of formula I AR is:
(1) an optionally substituted phenyl group: 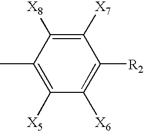
or
(2) an optionally substituted thiophene or furan group: 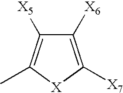
where X is S or O;
R₂ is hydrogen, an OR group, a halogen, an alkyl group, an alkoxy group, —CO—R group, —SR group, a cyano group, a nitro group, a thiol group, and a hydroxy group, where R is H, or an alkyl group; and
X₅—X₈, independently of one another, are selected from the group consisting of hydrogens, halogens, alkyl groups, alkoxy groups, thioalkoxyl groups, —CO—R groups, cyano groups, nitro groups, thiol groups, and hydroxy groups, where R is H, or a alkyl group.

16. The method of claim 14 wherein in the compound of formula I AR is an optionally substituted phenyl groups R₃ is a C1-C6 alkyl group, or a C2-C6 alkenyl groups which is optionally substituted with one or more halogens, one or more cyano or one or more nitro groups.

17. The method of claim 15 wherein R₁ is OH and R₂ is hydrogen or OR where R is hydrogen or a C1-C6 alkyl group.

18. The method of claim 14 wherein the disease, disorder, or condition is osteoporosis or symptoms thereof.

19. The method of claim 14 wherein the disease, disorder, or condition is hyperplasia or symptoms thereof.

20. The method of claim 14 wherein the disease, disorder, or condition is breast cancer or symptoms thereof.

21. The method of claim 14 wherein the disease, disorder, or condition is inflammation or symptoms thereof.

22. The method of claim 14 wherein the disease, disorder, or condition is cardiovascular disease or symptoms thereof.

23. The method of claim 14 wherein the disease, disorder, or condition is depression or anxiety or symptoms thereof.

24. The method of claim 14 wherein the disease, disorder, or condition is an endocrine disorder or symptoms thereof.

25. The method of claim 14 wherein the disease, disorder, or condition is an immune disorder or symptoms thereof.

26. The method of claim 14 wherein the disease, disorder, or condition is infertility or symptoms thereof.

27. An ER ligand of formula: 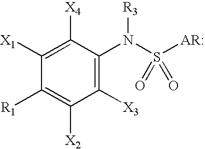
Wherein:
AR is an optionally substituted aryl group;
R₃ is an alkyl, alkenyl, alkynyl, benzyl, or phenyl group;
R₁ is a hydrogen, a halide, a hydroxy, thiol, an alkyl, alkenyl, alkynyl, benzyl, phenyl alkoxy, thioalkoxy, or aryloxy group; and
X₁—X₄, independently of one another, are selected from the group consisting of hydrogens, halogens, alkyl groups, alkoxy groups, —CO—R groups, —SR groups, cyano groups, nitro groups, hydroxy groups, alkoxy groups, thiol groups, and thioalkoxy groups, where R is H, or an alkyl group, wherein R₃ can be linked with X₃, or X₄ to form a 5, 6 or 7-member ring which may be an aromatic ring, or may contain one or two double bonds and wherein the ring optionally contains one or two additional heteroatoms wherein all alkyl, alkenyl, alkynyl, aryl, benzyl and phenyl groups are optional substituted and wherein optional substitution means substitution with one or more halogens, cyano groups, nitro groups, hydroxy groups, alkoxy groups, thiol groups, thioalkoxy groups, aryloxy groups, N(R)′₂ groups, CON(R′)₂ groups or —COOR′ groups, where R′ is H or an alkyl group and where R′ groups may be linked to form a cyclic alkyl group.

28. The compound of claim 27 wherein R₃ is a C1-C6 alkyl group or a C2-C6 alkenyl group which is optionally substituted with one or more halogens, one or more cyano groups or one or more nitro groups.

29. The compound of claim 27 wherein R₃ is a C1-C6 cycloalkyl group which is optionally substituted with one or more halogens, one or more cyano groups or one or more nitro groups.

30. The compound of claim 27 wherein AR is:
(1) an optionally substituted phenyl group: 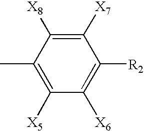
or
an optionally substituted thiophene or furan group: 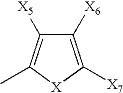
where X is S or O;
R₂ is hydrogen, an OR group, a halogen, an alkyl group, an alkoxy group, —CO—R group, —SR group, a cyano group, a nitro group, a thiol group, and a hydroxy group, where R is H, or an alkyl group; and
X₅—X₈, independently of one another, are selected from the group consisting of hydrogens, halogens, alkyl groups, alkoxy groups, thioalkoxyl groups, —CO—R groups, cyano groups, nitro groups, thiol groups, and hydroxy groups, where R is H, or a alkyl group.

31. The compound of claim 30 wherein R₃ is a C1-C6 alkyl group or a C2-C6 alkenyl group which is optionally substituted with one or more halogens, one or more cyano groups or one or more nitro groups.

32. The compound of claim 30 wherein R₃ is a C1-C6 cycloalkyl group which is optionally substituted with one or more halogens, one or more cyano groups or one or more nitro groups.

33. The compound of claim 30 wherein R₁ and R₂ are OH groups.

34. A pharmaceutical composition which comprises a pharmaceutically acceptable carrier and one or more compounds of formula I or a salt, stereoisomer or prodrug thereof present in the composition in an amount or a combined amount effective for treating a disease, condition, disorder or symptoms affect by ER wherein formula I is: 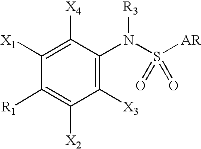
wherein:
AR is an optionally substituted aryl group;
R₃ is an alkyl, alkenyl, alkynyl, benzyl, or phenyl group;
R₁ is a hydrogen, a halide, a hydroxy, thiol, an alkyl, alkenyl, alkynyl, benzyl, phenyl alkoxy, thioalkoxy, or aryloxy group; and
X₁—X₄, independently of one another, are selected from the group consisting of hydrogens, halogens, alkyl groups, alkoxy groups, —CO—R groups, —SR groups, cyano groups, nitro groups, hydroxy groups, alkoxy groups, thiol groups, and thioalkoxy groups, where R is H, or an alkyl group, wherein R₃ can be linked with X₃, or X₄ to form a 5, 6 or 7-member ring which may be an aromatic ring, or may contain one or two double bonds and wherein the ring optionally contains one or two additional heteroatoms wherein all alkyl, alkenyl, alkynyl, aryl, benzyl and phenyl groups are optional substituted and wherein optional substitution means substitution with one or more halogens, cyano groups, nitro groups, hydroxy groups, alkoxy groups, thiol groups, thioalkoxy groups, aryloxy groups, N(R)′₂ groups, CON(R′)₂ groups or —COOR′ groups, where R′ is H or an alkyl group and where R′ groups may be linked to form a cyclic alkyl group.

35. The composition of claim 34 wherein the compound of formula I or a salt, stereoisomer, or prodrug thereof is present in an amount or a combined amount effective for effective for treating a disease, condition, disorder or symptoms affected by ER beta.
